SEQUENCE LISTING (379 sequences)

SEQ ID NO: 1 - RNA, 21 nt, Homo sapiens
cugaccuaugaauugacagcc

SEQ ID NO: 2 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 3 - RNA, 12 nt, Artificial Sequence
  Synthetic
cugaccuaugaa

SEQ ID NO: 4 - RNA, 21 nt, Homo sapiens
augaccuaugaauugacagac

SEQ ID NO: 5 - RNA, 110 nt, Homo sapiens
aucauucagaaaugguauacaggaaaaugaccuaugaauugacagacaauauagcugaguuugucugucauuucuuuaggccaauauucuguaugacugugcuacuucaa

SEQ ID NO: 6 - RNA, 12 nt, Artificial Sequence
  Synthetic
augaccuaugaa

SEQ ID NO: 7 - RNA, 21 nt, Artificial Sequence
  Synthetic
cugucaauucaucggucugau

SEQ ID NO: 8 - RNA, 21 nt, Artificial Sequence
  Synthetic
cugcacuaugaauugacagcc

SEQ ID NO: 9 - RNA, 21 nt, Artificial Sequence
  Synthetic
cugucaauucauagugcugau

SEQ ID NO: 10 - RNA, 21 nt, Artificial Sequence
  Synthetic
cugucaauucauaggucuuau

SEQ ID NO: 11 - DNA, 21 bp, Artificial Sequence
  Synthetic
ucgaaguauuccgcguacgtt

SEQ ID NO: 12 - DNA, 21 bp, Artificial Sequence
  Synthetic
cguacgcggaauacuucgatt

SEQ ID NO: 13 - DNA, 21 bp, Artificial Sequence
  Synthetic
auggaugcgagaaucauggtt

SEQ ID NO: 14 - DNA, 21 bp, Artificial Sequence
  Synthetic
ccaugauucucgcauccautt

SEQ ID NO: 15 - DNA, 21 bp, Artificial Sequence
  Synthetic
uaccuugcugucaagguuctt

SEQ ID NO: 16 - DNA, 21 bp, Artificial Sequence
  Synthetic
gaaccuugacagcaagguatt

SEQ ID NO: 17 - DNA, 21 bp, Artificial Sequence
  Synthetic
uaucucggagguagcuuuctt

SEQ ID NO: 18 - DNA, 21 bp, Artificial Sequence
  Synthetic
gaaagcuaccuccgagauatt

SEQ ID NO: 19 - DNA, 21 bp, Artificial Sequence
  Synthetic
aucuuccggaacuuguccctt

SEQ ID NO: 20 - DNA, 21 bp, Artificial Sequence
  Synthetic
gggacaaguuccggaagautt

SEQ ID NO: 21 - DNA, 21 bp, Artificial Sequence
  Synthetic
uucccgcuguccgaagcugtt

SEQ ID NO: 22 - DNA, 21 bp, Artificial Sequence
  Synthetic
cagcuucggacagcgggaatt

SEQ ID NO: 23 - DNA, 21 bp, Artificial Sequence
  Synthetic
ugggcgugaacuuguaggctt

SEQ ID NO: 24 - DNA, 21 bp, Artificial Sequence
  Synthetic
gccuacaaguucacgcccatt

SEQ ID NO: 25 - DNA, 21 bp, Artificial Sequence
  Synthetic
ucccuuaggagcgaauaugtt

SEQ ID NO: 26 - DNA, 21 bp, Artificial Sequence
  Synthetic
cauauucgcuccuaagggatt

SEQ ID NO: 27 - DNA, 21 bp, Artificial Sequence
  Synthetic
uaauccaggccuguguuagtt

SEQ ID NO: 28 - DNA, 21 bp, Artificial Sequence
  Synthetic
cuaacacaggccuggauuatt

SEQ ID NO: 29 - DNA, 21 bp, Artificial Sequence
  Synthetic
uuggcuaucaccuaacauctt

SEQ ID NO: 30 - DNA, 21 bp, Artificial Sequence
  Synthetic
gauguuaggugauagccaatt

SEQ ID NO: 31 - DNA, 21 bp, Artificial Sequence
  Synthetic
uaacggcccaggagauggctt

SEQ ID NO: 32 - DNA, 21 bp, Artificial Sequence
  Synthetic
gccaucuccugggccguuatt

SEQ ID NO: 33 - DNA, 21 bp, Artificial Sequence
  Synthetic
aaauaaaccagcaccaagctt

SEQ ID NO: 34 - DNA, 21 bp, Artificial Sequence
  Synthetic
gcuuggugcugguuuauuutt

SEQ ID NO: 35 - DNA, 21 bp, Artificial Sequence
  Synthetic
acaggugcgaguuccugggtt

SEQ ID NO: 36 - DNA, 21 bp, Artificial Sequence
  Synthetic
cccaggaacucgcaccugutt

SEQ ID NO: 37 - DNA, 21 bp, Artificial Sequence
  Synthetic
uagguccguagauauagggtt

SEQ ID NO: 38 - DNA, 21 bp, Artificial Sequence
  Synthetic
cccuauaucuacggaccuatt

SEQ ID NO: 39 - DNA, 21 bp, Artificial Sequence
  Synthetic
uucuuaagcggcauucucgtt

SEQ ID NO: 40 - DNA, 21 bp, Artificial Sequence
  Synthetic
cgagaaugccgcuuaagaatt

SEQ ID NO: 41 - DNA, 21 bp, Artificial Sequence
  Synthetic
ucaaacccagcuuacagugtt

SEQ ID NO: 42 - DNA, 21 bp, Artificial Sequence
  Synthetic
cacuguaagcuggguuugatt

SEQ ID NO: 43 - DNA, 21 bp, Artificial Sequence
  Synthetic
ugaacaguccuuaugauuctt

SEQ ID NO: 44 - DNA, 21 bp, Artificial Sequence
  Synthetic
gaaucauaaggacuguucatt

SEQ ID NO: 45 - DNA, 21 bp, Artificial Sequence
  Synthetic
ucacuagucccaugaucuctt

SEQ ID NO: 46 - DNA, 21 bp, Artificial Sequence
  Synthetic
gagaucaugggacuagugatt

SEQ ID NO: 47 - DNA, 21 bp, Artificial Sequence
  Synthetic
ugacuacugagcaauuaagtt

SEQ ID NO: 48 - DNA, 21 bp, Artificial Sequence
  Synthetic
cuuaauugcucaguagucatt

SEQ ID NO: 49 - DNA, 21 bp, Artificial Sequence
  Synthetic
uuugauuaaacuuuaguuctt

SEQ ID NO: 50 - DNA, 21 bp, Artificial Sequence
  Synthetic
gaacuaaaguuuaaucaaatt

SEQ ID NO: 51 - DNA, 21 bp, Artificial Sequence
  Synthetic
ucugaaagcgcucucuuuctt

SEQ ID NO: 52 - DNA, 21 bp, Artificial Sequence
  Synthetic
gaaagagagcgcuuucagatt

SEQ ID NO: 53 - DNA, 21 bp, Artificial Sequence
  Synthetic
aacuuccauaaagaguauctt

SEQ ID NO: 54 - DNA, 21 bp, Artificial Sequence
  Synthetic
gauacucuuuauggaaguutt

SEQ ID NO: 55 - DNA, 21 bp, Artificial Sequence
  Synthetic
aacuacaugaucauucuuctt

SEQ ID NO: 56 - DNA, 21 bp, Artificial Sequence
  Synthetic
gaagaaugaucauguaguutt

SEQ ID NO: 57 - DNA, 21 bp, Artificial Sequence
  Synthetic
ucccaugacauuaucuugctt

SEQ ID NO: 58 - DNA, 21 bp, Artificial Sequence
  Synthetic
gcaagauaaugucaugggatt

SEQ ID NO: 59 - DNA, 21 bp, Artificial Sequence
  Synthetic
aguacauccacagucuuugtt

SEQ ID NO: 60 - DNA, 21 bp, Artificial Sequence
  Synthetic
caaagacuguggauguacutt

SEQ ID NO: 61 - DNA, 21 bp, Artificial Sequence
  Synthetic
augaugacgcgcuggucugtt

SEQ ID NO: 62 - DNA, 21 bp, Artificial Sequence
  Synthetic
cagaccagcgcgucaucautt

SEQ ID NO: 63 - DNA, 21 bp, Artificial Sequence
  Synthetic
acaugucccacucaaacugtt

SEQ ID NO: 64 - DNA, 21 bp, Artificial Sequence
  Synthetic
caguuugagugggacaugutt

SEQ ID NO: 65 - DNA, 21 bp, Artificial Sequence
  Synthetic
uucaaauccagaucgucactt

SEQ ID NO: 66 - DNA, 21 bp, Artificial Sequence
  Synthetic
gugacgaucuggauuugaatt

SEQ ID NO: 67 - DNA, 21 bp, Artificial Sequence
  Synthetic
uaguaguaaaugaacgaagtt

SEQ ID NO: 68 - DNA, 21 bp, Artificial Sequence
  Synthetic
cuucguucauuuacuacuatt

SEQ ID NO: 69 - DNA, 21 bp, Artificial Sequence
  Synthetic
ugaacaagaaacuuccaactt

SEQ ID NO: 70 - DNA, 21 bp, Artificial Sequence
  Synthetic
guuggaaguuucuuguucatt

SEQ ID NO: 71 - DNA, 21 bp, Artificial Sequence
  Synthetic
ucaccguagcugugaucugtt

SEQ ID NO: 72 - DNA, 21 bp, Artificial Sequence
  Synthetic
cagaucacagcuacggugatt

SEQ ID NO: 73 - DNA, 21 bp, Artificial Sequence
  Synthetic
uauaauucuuacguaaauctt

SEQ ID NO: 74 - DNA, 21 bp, Artificial Sequence
  Synthetic
gauuuacguaagaauuauatt

SEQ ID NO: 75 - DNA, 21 bp, Artificial Sequence
  Synthetic
ucuauaauucuguugagugtt

SEQ ID NO: 76 - DNA, 21 bp, Artificial Sequence
  Synthetic
cacucaacagaauuauagatt

SEQ ID NO: 77 - DNA, 21 bp, Artificial Sequence
  Synthetic
ucgauaagcaguauaauuctt

SEQ ID NO: 78 - DNA, 21 bp, Artificial Sequence
  Synthetic
gaauuauacugcuuaucgatt

SEQ ID NO: 79 - DNA, 21 bp, Artificial Sequence
  Synthetic
ucacacaugagcaucucuctt

SEQ ID NO: 80 - DNA, 21 bp, Artificial Sequence
  Synthetic
gagagaugcucaugugugatt

SEQ ID NO: 81 - DNA, 21 bp, Artificial Sequence
  Synthetic
uuuacggaaauccucaaagtt

SEQ ID NO: 82 - DNA, 21 bp, Artificial Sequence
  Synthetic
cuuugaggauuuccguaaatt

SEQ ID NO: 83 - DNA, 21 bp, Artificial Sequence
  Synthetic
ugucacugggucuggauugtt

SEQ ID NO: 84 - DNA, 21 bp, Artificial Sequence
  Synthetic
caauccagacccagugacatt

SEQ ID NO: 85 - DNA, 21 bp, Artificial Sequence
  Synthetic
acgaagaucauucaguuuctt

SEQ ID NO: 86 - DNA, 21 bp, Artificial Sequence
  Synthetic
gaaacugaaugaucuucgutt

SEQ ID NO: 87 - DNA, 21 bp, Artificial Sequence
  Synthetic
augucacccaaucuauuuctt

SEQ ID NO: 88 - DNA, 21 bp, Artificial Sequence
  Synthetic
gaaauagauugggugacautt

SEQ ID NO: 89 - DNA, 21 bp, Artificial Sequence
  Synthetic
aucccacuagguuuguucctt

SEQ ID NO: 90 - DNA, 21 bp, Artificial Sequence
  Synthetic
ggaacaaaccuagugggautt

SEQ ID NO: 91 - DNA, 21 bp, Artificial Sequence
  Synthetic
ucgggucccaucgaagucctt

SEQ ID NO: 92 - DNA, 21 bp, Artificial Sequence
  Synthetic
ggacuucgaugggacccgatt

SEQ ID NO: 93 - DNA, 21 bp, Artificial Sequence
  Synthetic
ugcucgaaaggaauaucuctt

SEQ ID NO: 94 - DNA, 21 bp, Artificial Sequence
  Synthetic
gagauauuccuuucgagcatt

SEQ ID NO: 95 - DNA, 21 bp, Artificial Sequence
  Synthetic
agcaggaccacuuccauggtt

SEQ ID NO: 96 - DNA, 21 bp, Artificial Sequence
  Synthetic
ccauggaagugguccugcutt

SEQ ID NO: 97 - DNA, 21 bp, Artificial Sequence
  Synthetic
uagcgugcggagcaauguctt

SEQ ID NO: 98 - DNA, 21 bp, Artificial Sequence
  Synthetic
gacauugcuccgcacgcuatt

SEQ ID NO: 99 - DNA, 21 bp, Artificial Sequence
  Synthetic
ugcguuccgccuugaacugtt

SEQ ID NO: 100 - DNA, 21 bp, Artificial Sequence
  Synthetic
caguucaaggcggaacgcatt

SEQ ID NO: 101 - DNA, 21 bp, Artificial Sequence
  Synthetic
uucauucagagacuuguugtt

SEQ ID NO: 102 - DNA, 21 bp, Artificial Sequence
  Synthetic
caacaagucucugaaugaatt

SEQ ID NO: 103 - DNA, 21 bp, Artificial Sequence
  Synthetic
ucaaagaaggccacaaucctt

SEQ ID NO: 104 - DNA, 21 bp, Artificial Sequence
  Synthetic
ggauuguggccuucuuugatt

SEQ ID NO: 105 - DNA, 21 bp, Artificial Sequence
  Synthetic
uuguggcccagauaggcactt

SEQ ID NO: 106 - DNA, 21 bp, Artificial Sequence
  Synthetic
gugccuaucugggccacaatt

SEQ ID NO: 107 - DNA, 21 bp, Artificial Sequence
  Synthetic
augcaagugaaugaacacctt

SEQ ID NO: 108 - DNA, 21 bp, Artificial Sequence
  Synthetic
gguguucauucacuugcautt

SEQ ID NO: 109 - DNA, 21 bp, Artificial Sequence
  Synthetic
uacuaaagaccauaaaguctt

SEQ ID NO: 110 - DNA, 21 bp, Artificial Sequence
  Synthetic
gacuuuauggucuuuaguatt

SEQ ID NO: 111 - DNA, 21 bp, Artificial Sequence
  Synthetic
uaccauuaggaacuuuguctt

SEQ ID NO: 112 - DNA, 21 bp, Artificial Sequence
  Synthetic
gacaaaguuccuaaugguatt

SEQ ID NO: 113 - DNA, 21 bp, Artificial Sequence
  Synthetic
agaugucuaauauaagacgtt

SEQ ID NO: 114 - DNA, 21 bp, Artificial Sequence
  Synthetic
cgucuuauauuagacaucutt

SEQ ID NO: 115 - DNA, 21 bp, Artificial Sequence
  Synthetic
ugacuacggugaugaccugtt

SEQ ID NO: 116 - DNA, 21 bp, Artificial Sequence
  Synthetic
caggucaucaccguagucatt

SEQ ID NO: 117 - DNA, 21 bp, Artificial Sequence
  Synthetic
uuguggcucuucuuagacctt

SEQ ID NO: 118 - DNA, 21 bp, Artificial Sequence
  Synthetic
ggucuaagaagagccacaatt

SEQ ID NO: 119 - DNA, 21 bp, Artificial Sequence
  Synthetic
gaccuuucgggagacuuugtt

SEQ ID NO: 120 - DNA, 21 bp, Artificial Sequence
  Synthetic
caaagucucccgaaagguctt

SEQ ID NO: 121 - DNA, 21 bp, Artificial Sequence
  Synthetic
uugcauggaagccauuguctt

SEQ ID NO: 122 - DNA, 21 bp, Artificial Sequence
  Synthetic
gacaauggcuuccaugcaatt

SEQ ID NO: 123 - DNA, 21 bp, Artificial Sequence
  Synthetic
uuaguagccaggacaguagtt

SEQ ID NO: 124 - DNA, 21 bp, Artificial Sequence
  Synthetic
cuacuguccuggcuacuaatt

SEQ ID NO: 125 - DNA, 21 bp, Artificial Sequence
  Synthetic
ugaauagaaagaauagggctt

SEQ ID NO: 126 - DNA, 21 bp, Artificial Sequence
  Synthetic
gcccuauucuuucuauucatt

SEQ ID NO: 127 - DNA, 21 bp, Artificial Sequence
  Synthetic
acauaacaggcuaucaaugtt

SEQ ID NO: 128 - DNA, 21 bp, Artificial Sequence
  Synthetic
cauugauagccuguuaugutt

SEQ ID NO: 129 - DNA, 21 bp, Artificial Sequence
  Synthetic
uuguuuagccucacaacugtt

SEQ ID NO: 130 - DNA, 21 bp, Artificial Sequence
  Synthetic
caguugugaggcuaaacaatt

SEQ ID NO: 131 - DNA, 21 bp, Artificial Sequence
  Synthetic
auaguggguauuuacugugtt

SEQ ID NO: 132 - DNA, 21 bp, Artificial Sequence
  Synthetic
cacaguaaauacccacuautt

SEQ ID NO: 133 - DNA, 21 bp, Artificial Sequence
  Synthetic
aaagaccuaaucuguucagtt

SEQ ID NO: 134 - DNA, 21 bp, Artificial Sequence
  Synthetic
cugaacagauuaggucuuutt

SEQ ID NO: 135 - DNA, 21 bp, Artificial Sequence
  Synthetic
uucgaucaggucuucuaactt

SEQ ID NO: 136 - DNA, 21 bp, Artificial Sequence
  Synthetic
guuagaagaccugaucgaatt

SEQ ID NO: 137 - DNA, 21 bp, Artificial Sequence
  Synthetic
ucuucauccucaauucgugtt

SEQ ID NO: 138 - DNA, 21 bp, Artificial Sequence
  Synthetic
cacgaauugaggaugaagatt

SEQ ID NO: 139 - DNA, 21 bp, Artificial Sequence
  Synthetic
ucuucguaaccuuuaccagtt

SEQ ID NO: 140 - DNA, 21 bp, Artificial Sequence
  Synthetic
cugguaaagguuacgaagatt

SEQ ID NO: 141 - DNA, 21 bp, Artificial Sequence
  Synthetic
augcuuauauaaacuagactt

SEQ ID NO: 142 - DNA, 21 bp, Artificial Sequence
  Synthetic
gucuaguuuauauaagcautt

SEQ ID NO: 143 - DNA, 21 bp, Artificial Sequence
  Synthetic
aaucagagccgauacugagtt

SEQ ID NO: 144 - DNA, 21 bp, Artificial Sequence
  Synthetic
cucaguaucggcucugauutt

SEQ ID NO: 145 - DNA, 21 bp, Artificial Sequence
  Synthetic
uugccuugcucaauauaggtt

SEQ ID NO: 146 - DNA, 21 bp, Artificial Sequence
  Synthetic
ccuauauugagcaaggcaatt

SEQ ID NO: 147 - DNA, 21 bp, Artificial Sequence
  Synthetic
uaccacuuuggguaaaccgtt

SEQ ID NO: 148 - DNA, 21 bp, Artificial Sequence
  Synthetic
cgguuuacccaaagugguatt

SEQ ID NO: 149 - DNA, 21 bp, Artificial Sequence
  Synthetic
uggcggaaggucuuaucgctt

SEQ ID NO: 150 - DNA, 21 bp, Artificial Sequence
  Synthetic
gcgauaagaccuuccgccatt

SEQ ID NO: 151 - DNA, 21 bp, Artificial Sequence
  Synthetic
aaucgucucccuuucccggtt

SEQ ID NO: 152 - DNA, 21 bp, Artificial Sequence
  Synthetic
ccgggaaagggagacgauutt

SEQ ID NO: 153 - DNA, 21 bp, Artificial Sequence
  Synthetic
gaacucuccagcucgaggctt

SEQ ID NO: 154 - DNA, 21 bp, Artificial Sequence
  Synthetic
gccucgagcuggagaguuctt

SEQ ID NO: 155 - DNA, 21 bp, Artificial Sequence
  Synthetic
acaccuggaaacuccuaggtt

SEQ ID NO: 156 - DNA, 21 bp, Artificial Sequence
  Synthetic
ccuaggaguuuccaggugutt

SEQ ID NO: 157 - DNA, 21 bp, Artificial Sequence
  Synthetic
augggaacgagucaucuuctt

SEQ ID NO: 158 - DNA, 21 bp, Artificial Sequence
  Synthetic
gaagaugacucguucccautt

SEQ ID NO: 159 - DNA, 21 bp, Artificial Sequence
  Synthetic
acuaguucacuauuuacactt

SEQ ID NO: 160 - DNA, 21 bp, Artificial Sequence
  Synthetic
guguaaauagugaacuagutt

SEQ ID NO: 161 - DNA, 21 bp, Artificial Sequence
  Synthetic
auggaaagccacugaacugtt

SEQ ID NO: 162 - DNA, 21 bp, Artificial Sequence
  Synthetic
caguucaguggcuuuccautt

SEQ ID NO: 163 - DNA, 21 bp, Artificial Sequence
  Synthetic
aauagcauaauaacaguagtt

SEQ ID NO: 164 - DNA, 21 bp, Artificial Sequence
  Synthetic
cuacuguuauuaugcuauutt

SEQ ID NO: 165 - DNA, 21 bp, Artificial Sequence
  Synthetic
uaaugacuaguguagauggtt

SEQ ID NO: 166 - DNA, 21 bp, Artificial Sequence
  Synthetic
ccaucuacacuagucauuatt

SEQ ID NO: 167 - DNA, 21 bp, Artificial Sequence
  Synthetic
uagcauaauaacaguagcgtt

SEQ ID NO: 168 - DNA, 21 bp, Artificial Sequence
  Synthetic
cgcuacuguuauuaugcuatt

SEQ ID NO: 169 - DNA, 21 bp, Artificial Sequence
  Synthetic
uacgucugcauuaugagagtt

SEQ ID NO: 170 - DNA, 21 bp, Artificial Sequence
  Synthetic
cucucauaaugcagacguatt

SEQ ID NO: 171 - DNA, 21 bp, Artificial Sequence
  Synthetic
uaaucgaagcacucgaauctt

SEQ ID NO: 172 - DNA, 21 bp, Artificial Sequence
  Synthetic
gauucgagugcuucgauuatt

SEQ ID NO: 173 - DNA, 21 bp, Artificial Sequence
  Synthetic
acgaggaugaccaccaaagtt

SEQ ID NO: 174 - DNA, 21 bp, Artificial Sequence
  Synthetic
cuuugguggucauccucgutt

SEQ ID NO: 175 - DNA, 21 bp, Artificial Sequence
  Synthetic
uccaaacugagcuauacactt

SEQ ID NO: 176 - DNA, 21 bp, Artificial Sequence
  Synthetic
guguauagcucaguuuggatt

SEQ ID NO: 177 - DNA, 21 bp, Artificial Sequence
  Synthetic
ugaccaauuauccaaacugtt

SEQ ID NO: 178 - DNA, 21 bp, Artificial Sequence
  Synthetic
caguuuggauaauuggucatt

SEQ ID NO: 179 - DNA, 21 bp, Artificial Sequence
  Synthetic
ucaugugaaaugagauuagtt

SEQ ID NO: 180 - DNA, 21 bp, Artificial Sequence
  Synthetic
cuaaucucauuucacaugatt

SEQ ID NO: 181 - DNA, 21 bp, Artificial Sequence
  Synthetic
ucuauuagguuaauucgactt

SEQ ID NO: 182 - DNA, 21 bp, Artificial Sequence
  Synthetic
gucgaauuaaccuaauagatt

SEQ ID NO: 183 - DNA, 21 bp, Artificial Sequence
  Synthetic
ucccgcuugauuuagauugtt

SEQ ID NO: 184 - DNA, 21 bp, Artificial Sequence
  Synthetic
caaucuaaaucaagcgggatt

SEQ ID NO: 185 - DNA, 21 bp, Artificial Sequence
  Synthetic
acgaacccgaauagaagugtt

SEQ ID NO: 186 - DNA, 21 bp, Artificial Sequence
  Synthetic
cacuucuauucggguucgutt

SEQ ID NO: 187 - DNA, 21 bp, Artificial Sequence
  Synthetic
uuucgacuaccauuuggugtt

SEQ ID NO: 188 - DNA, 21 bp, Artificial Sequence
  Synthetic
caccaaaugguagucgaaatt

SEQ ID NO: 189 - DNA, 21 bp, Artificial Sequence
  Synthetic
aauuaguuuaguuucaauctt

SEQ ID NO: 190 - DNA, 21 bp, Artificial Sequence
  Synthetic
gauugaaacuaaacuaauutt

SEQ ID NO: 191 - DNA, 21 bp, Artificial Sequence
  Synthetic
ucgauauggaaccaucuugtt

SEQ ID NO: 192 - DNA, 21 bp, Artificial Sequence
  Synthetic
caagaugguuccauaucgatt

SEQ ID NO: 193 - DNA, 21 bp, Artificial Sequence
  Synthetic
uccaucgagcauugaucuctt

SEQ ID NO: 194 - DNA, 21 bp, Artificial Sequence
  Synthetic
gagaucaaugcucgauggatt

SEQ ID NO: 195 - DNA, 21 bp, Artificial Sequence
  Synthetic
uaauugugguaucauacactt

SEQ ID NO: 196 - DNA, 21 bp, Artificial Sequence
  Synthetic
guguaugauaccacaauuatt

SEQ ID NO: 197 - DNA, 21 bp, Artificial Sequence
  Synthetic
ucaaguaguugauuauaagtt

SEQ ID NO: 198 - DNA, 21 bp, Artificial Sequence
  Synthetic
cuuauaaucaacuacuugatt

SEQ ID NO: 199 - DNA, 21 bp, Artificial Sequence
  Synthetic
agaauaacgucgcucuaugtt

SEQ ID NO: 200 - DNA, 21 bp, Artificial Sequence
  Synthetic
cauagagcgacguuauucutt

SEQ ID NO: 201 - DNA, 21 bp, Artificial Sequence
  Synthetic
aacuagagaacauuuagugtt

SEQ ID NO: 202 - DNA, 21 bp, Artificial Sequence
  Synthetic
cacuaaauguucucuaguutt

SEQ ID NO: 203 - DNA, 21 bp, Artificial Sequence
  Synthetic
aacauuacaggcaaucaggtt

SEQ ID NO: 204 - DNA, 21 bp, Artificial Sequence
  Synthetic
ccugauugccuguaauguutt

SEQ ID NO: 205 - DNA, 21 bp, Artificial Sequence
  Synthetic
auagaacuccucuugaaugtt

SEQ ID NO: 206 - DNA, 21 bp, Artificial Sequence
  Synthetic
cauucaagaggaguucuautt

SEQ ID NO: 207 - DNA, 21 bp, Artificial Sequence
  Synthetic
ugaaugcaaaucuacuaugtt

SEQ ID NO: 208 - DNA, 21 bp, Artificial Sequence
  Synthetic
cauaguagauuugcauucatt

SEQ ID NO: 209 - DNA, 21 bp, Artificial Sequence
  Synthetic
ucagccaacaucaaagcuctt

SEQ ID NO: 210 - DNA, 21 bp, Artificial Sequence
  Synthetic
gagcuuugauguuggcugatt

SEQ ID NO: 211 - DNA, 21 bp, Artificial Sequence
  Synthetic
aauacacugccgucuuaagtt

SEQ ID NO: 212 - DNA, 21 bp, Artificial Sequence
  Synthetic
cuuaagacggcaguguauutt

SEQ ID NO: 213 - DNA, 21 bp, Artificial Sequence
  Synthetic
ucccuaaacgcguugaugctt

SEQ ID NO: 214 - DNA, 21 bp, Artificial Sequence
  Synthetic
gcaucaacgcguuuagggatt

SEQ ID NO: 215 - DNA, 21 bp, Artificial Sequence
  Synthetic
ugaaauuauguaauauugctt

SEQ ID NO: 216 - DNA, 21 bp, Artificial Sequence
  Synthetic
gcaauauuacauaauuucatt

SEQ ID NO: 217 - DNA, 21 bp, Artificial Sequence
  Synthetic
uucccaaacuggauagcuctt

SEQ ID NO: 218 - DNA, 21 bp, Artificial Sequence
  Synthetic
gagcuauccaguuugggaatt

SEQ ID NO: 219 - DNA, 21 bp, Artificial Sequence
  Synthetic
aaucagaaccuuucaaguctt

SEQ ID NO: 220 - DNA, 21 bp, Artificial Sequence
  Synthetic
gacuugaaagguucugauutt

SEQ ID NO: 221 - DNA, 21 bp, Artificial Sequence
  Synthetic
ugagauuucuucuucaauctt

SEQ ID NO: 222 - DNA, 21 bp, Artificial Sequence
  Synthetic
gauugaagaagaaaucucatt

SEQ ID NO: 223 - DNA, 21 bp, Artificial Sequence
  Synthetic
uaugccagauggauuucugtt

SEQ ID NO: 224 - DNA, 21 bp, Artificial Sequence
  Synthetic
cagaaauccaucuggcauatt

SEQ ID NO: 225 - DNA, 21 bp, Artificial Sequence
  Synthetic
uuagccacucuucuaguuctt

SEQ ID NO: 226 - DNA, 21 bp, Artificial Sequence
  Synthetic
gaacuagaagaguggcuaatt

SEQ ID NO: 227 - DNA, 21 bp, Artificial Sequence
  Synthetic
uuaacacagcaugauuucgtt

SEQ ID NO: 228 - DNA, 21 bp, Artificial Sequence
  Synthetic
cgaaaucaugcuguguuaatt

SEQ ID NO: 229 - DNA, 21 bp, Artificial Sequence
  Synthetic
uugcgucgaggccuaaagctt

SEQ ID NO: 230 - DNA, 21 bp, Artificial Sequence
  Synthetic
gcuuuaggccucgacgcaatt

SEQ ID NO: 231 - DNA, 21 bp, Artificial Sequence
  Synthetic
auguugugaugucuaaucctt

SEQ ID NO: 232 - DNA, 21 bp, Artificial Sequence
  Synthetic
ggauuagacaucacaacautt

SEQ ID NO: 233 - DNA, 21 bp, Artificial Sequence
  Synthetic
aauaguucuguguaagugctt

SEQ ID NO: 234 - DNA, 21 bp, Artificial Sequence
  Synthetic
gcacuuacacagaacuauutt

SEQ ID NO: 235 - DNA, 21 bp, Artificial Sequence
  Synthetic
uuccaccagcacuuagcactt

SEQ ID NO: 236 - DNA, 21 bp, Artificial Sequence
  Synthetic
gugcuaagugcugguggaatt

SEQ ID NO: 237 - DNA, 21 bp, Artificial Sequence
  Synthetic
uuagacaccgccuucaauctt

SEQ ID NO: 238 - DNA, 21 bp, Artificial Sequence
  Synthetic
gauugaaggcggugucuaatt

SEQ ID NO: 239 - DNA, 21 bp, Artificial Sequence
  Synthetic
acuaaaugaagaaucuuggtt

SEQ ID NO: 240 - DNA, 21 bp, Artificial Sequence
  Synthetic
ccaagauucuucauuuagutt

SEQ ID NO: 241 - DNA, 21 bp, Artificial Sequence
  Synthetic
aaaguccucucgauacucctt

SEQ ID NO: 242 - DNA, 21 bp, Artificial Sequence
  Synthetic
ggaguaucgagaggacuuutt

SEQ ID NO: 243 - DNA, 21 bp, Artificial Sequence
  Synthetic
ucacggugagcacaaaguctt

SEQ ID NO: 244 - DNA, 21 bp, Artificial Sequence
  Synthetic
gacuuugugcucaccgugatt

SEQ ID NO: 245 - DNA, 21 bp, Artificial Sequence
  Synthetic
agacgccagacuuugucugtt

SEQ ID NO: 246 - DNA, 21 bp, Artificial Sequence
  Synthetic
cagacaaagucuggcgucutt

SEQ ID NO: 247 - DNA, 21 bp, Artificial Sequence
  Synthetic
ugucauugcuucuucaucgtt

SEQ ID NO: 248 - DNA, 21 bp, Artificial Sequence
  Synthetic
cgaugaagaagcaaugacatt

SEQ ID NO: 249 - DNA, 21 bp, Artificial Sequence
  Synthetic
aggacacucggaugaucugtt

SEQ ID NO: 250 - DNA, 21 bp, Artificial Sequence
  Synthetic
cagaucauccgaguguccutt

SEQ ID NO: 251 - DNA, 21 bp, Artificial Sequence
  Synthetic
ucuguccuguaguaaagggtt

SEQ ID NO: 252 - DNA, 21 bp, Artificial Sequence
  Synthetic
cccuuuacuacaggacagatt

SEQ ID NO: 253 - DNA, 21 bp, Artificial Sequence
  Synthetic
uaaggugugcguuucguuctt

SEQ ID NO: 254 - DNA, 21 bp, Artificial Sequence
  Synthetic
gaacgaaacgcacaccuuatt

SEQ ID NO: 255 - DNA, 21 bp, Artificial Sequence
  Synthetic
acaguccgcacauaacauctt

SEQ ID NO: 256 - DNA, 21 bp, Artificial Sequence
  Synthetic
gauguuaugugcggacugutt

SEQ ID NO: 257 - DNA, 21 bp, Artificial Sequence
  Synthetic
uccaucgaggcugaaaucctt

SEQ ID NO: 258 - DNA, 21 bp, Artificial Sequence
  Synthetic
ggauuucagccucgauggatt

SEQ ID NO: 259 - DNA, 21 bp, Artificial Sequence
  Synthetic
aucuuaauagucuugucuctt

SEQ ID NO: 260 - DNA, 21 bp, Artificial Sequence
  Synthetic
gagacaagacuauuaagautt

SEQ ID NO: 261 - DNA, 21 bp, Artificial Sequence
  Synthetic
uugcuacgacccauacacgtt

SEQ ID NO: 262 - DNA, 21 bp, Artificial Sequence
  Synthetic
cguguaugggucguagcaatt

SEQ ID NO: 263 - DNA, 21 bp, Artificial Sequence
  Synthetic
auuuaaacagagcucaaugtt

SEQ ID NO: 264 - DNA, 21 bp, Artificial Sequence
  Synthetic
cauugagcucuguuuaaautt

SEQ ID NO: 265 - DNA, 21 bp, Artificial Sequence
  Synthetic
uuacucuaauuauucuacctt

SEQ ID NO: 266 - DNA, 21 bp, Artificial Sequence
  Synthetic
gguagaauaauuagaguaatt

SEQ ID NO: 267 - DNA, 21 bp, Artificial Sequence
  Synthetic
ugaaccaccauccuuuauctt

SEQ ID NO: 268 - DNA, 21 bp, Artificial Sequence
  Synthetic
gauaaaggauggugguucatt

SEQ ID NO: 269 - DNA, 21 bp, Artificial Sequence
  Synthetic
uuugcuauuggugauacagtt

SEQ ID NO: 270 - DNA, 21 bp, Artificial Sequence
  Synthetic
cuguaucaccaauagcaaatt

SEQ ID NO: 271 - DNA, 21 bp, Artificial Sequence
  Synthetic
uaacgcugguggauguaagtt

SEQ ID NO: 272 - DNA, 21 bp, Artificial Sequence
  Synthetic
cuuacauccaccagcguuatt

SEQ ID NO: 273 - DNA, 21 bp, Artificial Sequence
  Synthetic
uagcugagcuuauuccaugtt

SEQ ID NO: 274 - DNA, 21 bp, Artificial Sequence
  Synthetic
cauggaauaagcucagcuatt

SEQ ID NO: 275 - DNA, 21 bp, Artificial Sequence
  Synthetic
aaaguuaaugcaagcauugtt

SEQ ID NO: 276 - DNA, 21 bp, Artificial Sequence
  Synthetic
caaugcuugcauuaacuuutt

SEQ ID NO: 277 - DNA, 21 bp, Artificial Sequence
  Synthetic
uuggacaagcggcuuuaggtt

SEQ ID NO: 278 - DNA, 21 bp, Artificial Sequence
  Synthetic
ccuaaagccgcuuguccaatt

SEQ ID NO: 279 - DNA, 21 bp, Artificial Sequence
  Synthetic
uggacaagcggcuuuaggctt

SEQ ID NO: 280 - DNA, 21 bp, Artificial Sequence
  Synthetic
gccuaaagccgcuuguccatt

SEQ ID NO: 281 - DNA, 21 bp, Artificial Sequence
  Synthetic
uuggagaaguacuuguuggtt

SEQ ID NO: 282 - DNA, 21 bp, Artificial Sequence
  Synthetic
ccaacaaguacuucuccaatt

SEQ ID NO: 283 - DNA, 21 bp, Artificial Sequence
  Synthetic
uuuauauccugguaauggctt

SEQ ID NO: 284 - DNA, 21 bp, Artificial Sequence
  Synthetic
gccauuaccaggauauaaatt

SEQ ID NO: 285 - DNA, 21 bp, Artificial Sequence
  Synthetic
agacuaaagcuaaagucagtt

SEQ ID NO: 286 - DNA, 21 bp, Artificial Sequence
  Synthetic
cugacuuuagcuuuagucutt

SEQ ID NO: 287 - DNA, 21 bp, Artificial Sequence
  Synthetic
uaauucaacagcauaguugtt

SEQ ID NO: 288 - DNA, 21 bp, Artificial Sequence
  Synthetic
caacuaugcuguugaauuatt

SEQ ID NO: 289 - DNA, 21 bp, Artificial Sequence
  Synthetic
uugucuuugaacccauuuctt

SEQ ID NO: 290 - DNA, 21 bp, Artificial Sequence
  Synthetic
gaaauggguucaaagacaatt

SEQ ID NO: 291 - DNA, 21 bp, Artificial Sequence
  Synthetic
uaauucugccacaagaguctt

SEQ ID NO: 292 - DNA, 21 bp, Artificial Sequence
  Synthetic
gacucuuguggcagaauuatt

SEQ ID NO: 293 - DNA, 21 bp, Artificial Sequence
  Synthetic
acugagagggcauuuccactt

SEQ ID NO: 294 - DNA, 21 bp, Artificial Sequence
  Synthetic
guggaaaugcccucucagutt

SEQ ID NO: 295 - DNA, 21 bp, Artificial Sequence
  Synthetic
ucuuccuaaaguacuuucgtt

SEQ ID NO: 296 - DNA, 21 bp, Artificial Sequence
  Synthetic
cgaaaguacuuuaggaagatt

SEQ ID NO: 297 - DNA, 21 bp, Artificial Sequence
  Synthetic
ugagcuuuauuauugguactt

SEQ ID NO: 298 - DNA, 21 bp, Artificial Sequence
  Synthetic
guaccaauaauaaagcucatt

SEQ ID NO: 299 - DNA, 21 bp, Artificial Sequence
  Synthetic
uucuacaccagcuugucugtt

SEQ ID NO: 300 - DNA, 21 bp, Artificial Sequence
  Synthetic
cagacaagcugguguagaatt

SEQ ID NO: 301 - DNA, 21 bp, Artificial Sequence
  Synthetic
aaacucgccacaguaguugtt

SEQ ID NO: 302 - DNA, 21 bp, Artificial Sequence
  Synthetic
caacuacuguggcgaguuutt

SEQ ID NO: 303 - DNA, 21 bp, Artificial Sequence
  Synthetic
aucguagaaaccauagaugtt

SEQ ID NO: 304 - DNA, 21 bp, Artificial Sequence
  Synthetic
caucuaugguuucuacgautt

SEQ ID NO: 305 - DNA, 21 bp, Artificial Sequence
  Synthetic
aguuugauguuguagcguctt

SEQ ID NO: 306 - DNA, 21 bp, Artificial Sequence
  Synthetic
gacgcuacaacaucaaacutt

SEQ ID NO: 307 - DNA, 21 bp, Artificial Sequence
  Synthetic
aauaagaacuucaucuauctt

SEQ ID NO: 308 - DNA, 21 bp, Artificial Sequence
  Synthetic
gauagaugaaguucuuauutt

SEQ ID NO: 309 - DNA, 21 bp, Artificial Sequence
  Synthetic
acaagauaggacuaaugcctt

SEQ ID NO: 310 - DNA, 21 bp, Artificial Sequence
  Synthetic
ggcauuaguccuaucuugutt

SEQ ID NO: 311 - DNA, 21 bp, Artificial Sequence
  Synthetic
aaagucccuggcauaggactt

SEQ ID NO: 312 - DNA, 21 bp, Artificial Sequence
  Synthetic
guccuaugccagggacuuutt

SEQ ID NO: 313 - DNA, 21 bp, Artificial Sequence
  Synthetic
ugagcuaccaccugauuagtt

SEQ ID NO: 314 - DNA, 21 bp, Artificial Sequence
  Synthetic
cuaaucaggugguagcucatt

SEQ ID NO: 315 - DNA, 21 bp, Artificial Sequence
  Synthetic
aagauuguccaguagacagtt

SEQ ID NO: 316 - DNA, 21 bp, Artificial Sequence
  Synthetic
cugucuacuggacaaucuutt

SEQ ID NO: 317 - DNA, 21 bp, Artificial Sequence
  Synthetic
ugauauuuccuccaauaggtt

SEQ ID NO: 318 - DNA, 21 bp, Artificial Sequence
  Synthetic
ccuauuggaggaaauaucatt

SEQ ID NO: 319 - DNA, 21 bp, Artificial Sequence
  Synthetic
aacaugaucuccuuguugctt

SEQ ID NO: 320 - DNA, 21 bp, Artificial Sequence
  Synthetic
gcaacaaggagaucauguutt

SEQ ID NO: 321 - DNA, 21 bp, Artificial Sequence
  Synthetic
uaaaguccugcaguuugactt

SEQ ID NO: 322 - DNA, 21 bp, Artificial Sequence
  Synthetic
gucaaacugcaggacuuuatt

SEQ ID NO: 323 - DNA, 21 bp, Artificial Sequence
  Synthetic
ucaaggaacuccacgaaagtt

SEQ ID NO: 324 - DNA, 21 bp, Artificial Sequence
  Synthetic
cuuucguggaguuccuugatt

SEQ ID NO: 325 - DNA, 21 bp, Artificial Sequence
  Synthetic
uucgaguagaagucauuuctt

SEQ ID NO: 326 - DNA, 21 bp, Artificial Sequence
  Synthetic
gaaaugacuucuacucgaatt

SEQ ID NO: 327 - DNA, 21 bp, Artificial Sequence
  Synthetic
uccguaagggugaacuaggtt

SEQ ID NO: 328 - DNA, 21 bp, Artificial Sequence
  Synthetic
ccuaguucacccuuacggatt

SEQ ID NO: 329 - DNA, 21 bp, Artificial Sequence
  Synthetic
auuuauggacacugauuuctt

SEQ ID NO: 330 - DNA, 21 bp, Artificial Sequence
  Synthetic
gaaaucaguguccauaaautt

SEQ ID NO: 331 - DNA, 21 bp, Artificial Sequence
  Synthetic
uccuauaccaggguaguagtt

SEQ ID NO: 332 - DNA, 21 bp, Artificial Sequence
  Synthetic
cuacuacccugguauaggatt

SEQ ID NO: 333 - DNA, 21 bp, Artificial Sequence
  Synthetic
uaaaugcaacacuuacgagtt

SEQ ID NO: 334 - DNA, 21 bp, Artificial Sequence
  Synthetic
cucguaaguguugcauuuatt

SEQ ID NO: 335 - DNA, 21 bp, Artificial Sequence
  Synthetic
acgaggaguaguuaaugcctt

SEQ ID NO: 336 - DNA, 21 bp, Artificial Sequence
  Synthetic
ggcauuaacuacuccucgutt

SEQ ID NO: 337 - DNA, 21 bp, Artificial Sequence
  Synthetic
aucgcaaucacuuccuuugtt

SEQ ID NO: 338 - DNA, 21 bp, Artificial Sequence
  Synthetic
caaaggaagugauugcgautt

SEQ ID NO: 339 - DNA, 21 bp, Artificial Sequence
  Synthetic
uguuuagggccguagagactt

SEQ ID NO: 340 - DNA, 21 bp, Artificial Sequence
  Synthetic
gucucuacggcccuaaacatt

SEQ ID NO: 341 - DNA, 21 bp, Artificial Sequence
  Synthetic
uuccagaaaggguuauuactt

SEQ ID NO: 342 - DNA, 21 bp, Artificial Sequence
  Synthetic
guaauaacccuuucuggaatt

SEQ ID NO: 343 - DNA, 21 bp, Artificial Sequence
  Synthetic
agaagcaccauucuuaagctt

SEQ ID NO: 344 - DNA, 21 bp, Artificial Sequence
  Synthetic
gcuuaagaauggugcuucutt

SEQ ID NO: 345 - DNA, 21 bp, Artificial Sequence
  Synthetic
ucaacugaucucuuaaguctt

SEQ ID NO: 346 - DNA, 21 bp, Artificial Sequence
  Synthetic
gacuuaagagaucaguugatt

SEQ ID NO: 347 - DNA, 21 bp, Artificial Sequence
  Synthetic
aaauccaauaccauacuuctt

SEQ ID NO: 348 - DNA, 21 bp, Artificial Sequence
  Synthetic
gaaguaugguauuggauuutt

SEQ ID NO: 349 - DNA, 21 bp, Artificial Sequence
  Synthetic
uaaccacuuugauucaucgtt

SEQ ID NO: 350 - DNA, 21 bp, Artificial Sequence
  Synthetic
cgaugaaucaaagugguuatt

SEQ ID NO: 351 - DNA, 21 bp, Artificial Sequence
  Synthetic
auauaugucuuaauaugugtt

SEQ ID NO: 352 - DNA, 21 bp, Artificial Sequence
  Synthetic
cacauauuaagacauauautt

SEQ ID NO: 353 - DNA, 21 bp, Artificial Sequence
  Synthetic
uuaaacucagaacauaacctt

SEQ ID NO: 354 - DNA, 21 bp, Artificial Sequence
  Synthetic
gguuauguucugaguuuaatt

SEQ ID NO: 355 - DNA, 21 bp, Artificial Sequence
  Synthetic
acugguuccggaucuuguctt

SEQ ID NO: 356 - DNA, 21 bp, Artificial Sequence
  Synthetic
gacaagauccggaaccagutt

SEQ ID NO: 357 - DNA, 21 bp, Artificial Sequence
  Synthetic
uacaguugaugauuauauctt

SEQ ID NO: 358 - DNA, 21 bp, Artificial Sequence
  Synthetic
gauauaaucaucaacuguatt

SEQ ID NO: 359 - DNA, 21 bp, Artificial Sequence
  Synthetic
uagcaauccucucuuugugtt

SEQ ID NO: 360 - DNA, 21 bp, Artificial Sequence
  Synthetic
cacaaagagaggauugcuatt

SEQ ID NO: 361 - DNA, 21 bp, Artificial Sequence
  Synthetic
uggugcaagaaauuuauugtt

SEQ ID NO: 362 - DNA, 21 bp, Artificial Sequence
  Synthetic
caauaaauuucuugcaccatt

SEQ ID NO: 363 - DNA, 21 bp, Artificial Sequence
  Synthetic
uuuguaaugcucguugaagtt

SEQ ID NO: 364 - DNA, 21 bp, Artificial Sequence
  Synthetic
cuucaacgagcauuacaaatt

SEQ ID NO: 365 - DNA, 21 bp, Artificial Sequence
  Synthetic
ucucgaguaaacacaguugtt

SEQ ID NO: 366 - DNA, 21 bp, Artificial Sequence
  Synthetic
caacuguguuuacucgagatt

SEQ ID NO: 367 - DNA, 21 bp, Artificial Sequence
  Synthetic
uauaauaccugaaguauactt

SEQ ID NO: 368 - DNA, 21 bp, Artificial Sequence
  Synthetic
guauacuucagguauuauatt

SEQ ID NO: 369 - DNA, 21 bp, Artificial Sequence
  Synthetic
uauugccgagauuauucagtt

SEQ ID NO: 370 - DNA, 21 bp, Artificial Sequence
  Synthetic
cugaauaaucucggcaauatt

SEQ ID NO: 371 - DNA, 21 bp, Artificial Sequence
  Synthetic
ucuaauguaucacuuguagtt

SEQ ID NO: 372 - DNA, 21 bp, Artificial Sequence
  Synthetic
cuacaagugauacauuagatt

SEQ ID NO: 373 - DNA, 21 bp, Artificial Sequence
  Synthetic
uugauauugucuuugauugtt

SEQ ID NO: 374 - DNA, 21 bp, Artificial Sequence
  Synthetic
caaucaaagacaauaucaatt

SEQ ID NO: 375 - DNA, 21 bp, Artificial Sequence
  Synthetic
ucaaggaauucuuaaucactt

SEQ ID NO: 376 - DNA, 21 bp, Artificial Sequence
  Synthetic
gugauuaagaauuccuugatt

SEQ ID NO: 377 - DNA, 21 bp, Artificial Sequence
  Synthetic
ugucaacugagcauuaguctt

SEQ ID NO: 378 - DNA, 21 bp, Artificial Sequence
  Synthetic
gacuaaugcucaguugacatt

SEQ ID NO: 379 - DNA, 6 bp, Artificial Sequence
  Synthetic
aggtca

(19) United States
(12) Patent Application Publication
(10) Pub. No.: US 2011/0118337 A1
(43) Pub. Date: May 19, 2011

(54) Method of Using Compositions Comprising MIR-192 and/or MIR-215 for the Treatment of Cancer

(75) Inventors: Nelson Chau, San Diego, CA (US); Sara Georges, Boston, MA (US)

(73) Assignee: Merck Sharp & Dohme Corp.

(21) Appl. No.: 13/003,536

(22) PCT Filed: Jul. 9, 2009
(86) PCT No.: PCT/US09/50028

§ 371 Date: Jan. 10, 2011

Related U.S. Application Data
(60) Provisional application No. 61/079,771, filed on Jul. 10, 2008.

(51) Int. Cl. A61K 31/713; C12N 5/071; C07H 21/02; A61K 31/7105; A61P 35/00

(52) U.S. Cl. 514/44 A; 435/325; 536/23.1

(57) ABSTRACT

The invention provides methods and compositions for inhibiting the proliferation of mammalian cells. In some embodiments, the methods comprise contacting mammalian cells with an effective amount of at least one small interfering nucleic acid (siNA) agent that inhibits the level of expression of at least two miR 192 family responsive genes selected from the group consisting of SEPT 10, LMNB2, HRH1, HOXA10, ERCC3, MIS12, MPHOSPHI1, CDC7, SMARCB1, MAD2L1, DTL, RAC-GAP1, MCM10, PIM1, DLG5, BCL2, CUL5, and PRPF38A.

### RELATED APPLICATIONS

This application claims the benefit of U.S. Provisional Application No. 61/079,771, filed Jul. 10, 2008, the entire teachings of which are incorporated herein by reference.

### FIELD OF THE INVENTION

The invention generally relates to methods of using compositions comprising miR-192 and/or miR-215 and siRNAs for inhibiting miR-192 and/or miR-215 responsive target genes for the treatment of cancer.

### BACKGROUND

Many genes are related via common regulation, common functional molecular mechanisms, and common pathways. Understanding the relationship between genes is important for biological research and has extensive practical application in drug development and diagnostics.

MicroRNAs are a recently identified class of regulatory RNAs that target specific mRNAs for degradation or inhibition of translation, resulting in a decrease of the protein encoded by the target mRNA. Current estimates are that 30% or more of human mRNAs are regulated by miRNAs (Lewis et al., Cell 120:15-20 (2005)). Studies investigating expression profiles of various miRNAs in normal and cancer cells reveal that miRNA expression patterns may have clinical relevance. (See, e.g., Yanaihara, N., et al., Cancer Cell 9:189-198, 2006.) Application of various bioinformatics approaches have revealed that a single miRNA might bind to as many as 200 gene targets and these targets are often diverse in function, including, for example, transcription factors, secreted factors, receptors and transporters (see, e.g., Esquela-Kerscher and Slack, Nature Reviews 6:259-269 (2006); Bartel, D. P., et al., Nat Rev Genet 5(5):396-400 (2004)). Therefore, the deletion or overexpression of a particular miRNA is likely to be pleiotropic.

Events leading to the development of cancer from normal tissue have been well-charted, and a necessary step in this process is the dysregulation of cell cycle progression that facilitates the propagation and accumulation of genetic mutations. Within each cell, elaborate machinery exists to halt cell cycle progression in response to various stimuli, including DNA damage. Such regulation provides time for DNA repair prior to its replication and cell division, hence preserving the integrity of the genome. Multiple pathways lead to cell cycle arrest; however, the p53 tumor suppressor pathway has been extensively dissected and it has been shown that p53 activation leads to both G₁ and G₂/M arrest (Vousden, K. H., et al., Nat. Rev. Mol. Cell Biol. 8:275-283 (2007); Taylor, W. R., et al., Oncogene 20:1803-1815 (2001); Brown, L., et al., Crit. Rev. Eukaryot. Gene Expr. 17:73-85 (2007)). Although a number of key players in this pathway have been identified and characterized, the precise mechanism by which DNA damage leads to cell cycle arrest remains only partially understood.

Cell cycle arrest in response to DNA damage is an important anti-tumorigenic mechanism. microRNAs (miRNAs) have been shown recently to play key regulatory roles in cell cycle progression. miRNAs are abundant, ˜21 nucleotide non-coding RNAs that regulate the stability or translation of hundreds of mRNA targets in a sequence-specific manner. In doing so, miRNAs regulate key biological processes including cell growth, differentiation and death (Bartel, D. P., et al., Nat. Rev. Genet. 5:396-400 (2004)). Recently, new insight has been gained into the miRNA-mediated cell cycle regulation by identifying target transcripts of respective miRNAs (Carleton, M., et al., Cell Cycle 6:2127-2132 (2007); Johnson, C. D., et al., Cancer Res. 67:7713-7722 (2007); Ivanovsaka, I., et al., Mol. Cell Biol. 28:2167-2174 (2008)). For example, miR-34a is induced in response to p53 activation and mediates G₁ arrest by down-regulating multiple cell cycle-related transcripts.

While certain miRNAs exert their cell cycle effect through targeting key transcripts, other miRNAs do so through cooperatively down-regulating the expression of multiple cell cycle-related transcripts (He, L., et al., Nature 447:1130-1134 (2007); Linsley et al., Mol. Cell Biol. 27:2240-2252 (2007)). In addition to their effects on the cell cycle, these miRNAs and their family members are aberrantly expressed in human cancers suggesting a possible role in tumor suppression (Linsley et al., Mol. Cell Biol. 27:2240-2252 (2007); Calin, G. A., et al., Nat. Rev. Cancer 6:857-866 (2006); Takamizawa, J., et al., Cancer Res. 64:3753-3756 (2004); Inamura, K., et al., Lung Cancer 58:392-396 (2007); Cimminio, A., et al., PNAS 102:13944-13949 (2005); Ota, A., et al., Cancer Res. 64:3087-3095 (2004); He, L., et al., Nature 435:828-833 (2005)).

It is important to assign functions to miRNAs and to accurately identify miRNA responsive targets. Since a single miRNA can regulate hundreds of targets, understanding of biological pathways regulated by microRNAs is not obvious from examination of their targets. As functions are assigned to miRNAs, it is also important to determine which of their target(s) are responsible for a phenotype. It is also currently unknown whether the numerous miRNA responsive targets act individually or in concert.

There is growing realization that miRNAs, in addition to functioning as regulators of development, can act as oncogenes and tumor suppressors (Akao et al., 2006, Oncology Reports 16:845-50; Esquela-Kerscher and Slack, 2006, Nature Rev. 6:259-269; He et al., 2005, Nature 435:828-33) and that miRNA expression profiles can, under some circumstances, be used to diagnose and classify human cancers (Lu et al., 2005, Nature 435:834-38; Volinia et al., 2006, PNAS 103:2257-61; Yanaihara et al., 2006, Cancer Cell 9:189-198). Given the significance of TP53 in cancer and the importance of finding clinical biomarkers for TP53 status, there is need to identify RNA transcripts, including miRNAs, that are involved in regulation of the TP53 pathway.

### SUMMARY

In one aspect, the invention provides a method of inhibiting proliferation of a mammalian cell comprising introducing into the mammalian cell an effective amount of at least one small interfering nucleic acid (siNA) agent that inhibits the level of expression of at least one miR-192 family responsive gene comprising SEQ ID NO:379 in its 3′ untranslated region (3′UTR).

In another aspect, the invention provides a method of inhibiting cancer cell proliferation in a subject comprising contacting the cancer cells with an effective amount of at least one small interfering nucleic acid (siNA) agent that inhibits the level of expression of at least two miR-192 family responsive genes selected from the group consisting of SEPT 10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A, thereby inhibiting the proliferation of cancer cells in the subject.

In another aspect, the invention provides a composition comprising a combination of gene-specific agents directed to at least two miR-192 family responsive target genes selected from TABLE 3. In some embodiments, the compositions comprise gene-specific agents directed to at least two miR-192 family responsive genes are selected from the group consisting of SEPT 10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

In another aspect, the invention provides an isolated dsRNA molecule comprising one nucleotide strand that is substantially identical to a sequence selected from the group consisting of SEQ ID NO:13 to SEQ ID NO:120.

In yet another aspect, the invention provides a composition comprising at least one synthetic duplex microRNA mimetic and a delivery agent, the synthetic duplex microRNA mimetic(s) comprising: (i) a guide strand nucleic acid molecule consisting of a nucleotide sequence of 18 to 25 nucleotides, said guide strand nucleotide sequence comprising a seed region nucleotide sequence and a non-seed region nucleotide sequence, said seed region consisting essentially of nucleotide positions 1 to 12 and said non-seed region consisting essentially of nucleotide positions 13 to the 3′ end of said guide strand, wherein position 1 of said guide strand represents the 5′ end of said guide strand, wherein said seed region further comprises a consecutive nucleotide sequence of at least 6 nucleotides that is identical in sequence to a nucleotide sequence selected from the group consisting of SEQ ID NO:3 and SEQ ID NO:6; and (ii) a passenger strand nucleic acid molecule consisting of a nucleotide sequence of 18 to 25 nucleotides, said passenger strand comprising a nucleotide sequence that has at least one nucleotide sequence difference compared with the true reverse complement sequence of the seed region of the guide strand, wherein the at least one nucleotide difference is located within nucleotide position 13 to the 3′ end of said passenger strand.

The isolated nucleic acid molecules of the invention and compositions of the invention may be used for the methods of inhibiting proliferation of mammalian cells, such as for treatment of cancer in a mammalian subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the RNA sequences of miR-192 and miR-215 including corresponding “seed regions”;

FIG. 2A graphically illustrates the fold change (as compared to the untreated cells) of miR-192, miR-215 and miR-34a expression levels in either wild type A549 cells (p53+/+), or A549 (p53−/−) cells following treatment with 0, 10, 50 or 200 nM adriamycin, as described in Example 1;

FIG. 2B graphically illustrates the fold change (as compared to the untreated cells) in miR-192, miR-215 and miR-34a expression levels in either wild type TOV21G cells (p53+/+) or TOV21G (p53−/−) cells following treatment with 0, 10, 50 or 200 nM adriamycin, as described in Example 1;

FIG. 2C graphically illustrates the fold change (as compared to wild type untreated cells) of p21 expression levels in matched pairs of A549 cells and TOV21G cells wild type (p53+/+) or p53 kd−/− following treatment with 0, 10, 50 or 200 nM adriamycin, as described in Example 1;

FIG. 3A graphically illustrates the percentage of HCT116DICER^{ex5} cells in G1 after transfection with 10 mM miR-192 or 100 nM siRNA against luciferase, or 100 nM siRNA against the putative miR-192 target of interest, followed by treatment with nocodazole for an additional 18 hours prior to FACS analysis, as described in Example 4;

FIG. 3B graphically illustrates the percentage of HCT116DICER^{ex5} cells in G2 after transfection with 10 mM miR-192 or 100 nM siRNA against luciferase, or 100 nM siRNA against the putative miR-192 target of interest, followed by treatment with aphidicolin for an additional 18 hours prior to FACS analysis, as described in Example 4;

FIG. 4A graphically illustrates the transcript abundance (relative to a control luciferase siRNA) of a set of 18 candidate downstream targets of miR-192/miR-215 in U-2-OS cells transfected with miR-192 or a miR-192 with a seed region mutation, as described in Example 5;

FIG. 4B graphically illustrates the average normalized luciferase activity for each cell co-transfected with a reporter construct containing the 3′ UTR of a candidate gene fused to the luciferase open reading frame, and with either a miR-192 or miR-192 seed mutant, as measured in three separate trials conducted in duplicate. For each reporter construct, the luciferase activity of samples transfected with miR-192 mutant is set to a value of “1,” as described in Example 5;

FIG. 5A graphically illustrates the titration of siRNAs targeting miR-192 responsive genes in HCT116DICER^{ex5} cells after treatment with nocodazole that phenocopy miR-192 induced G1 arrest, as described in Example 6;

FIG. 5B graphically illustrates the titration of siRNAs targeting miR-192 responsive genes in HCT116DICER^{ex5} cells after treatment with aphidicolin that phenocopy miR-192 induced G2 arrest, as described in Example 6;

FIG. 6A graphically illustrates the results of cell cycle analysis of transfected HCT116DICER^{ex5} cells after treatment with nocodazole, wherein the cells were either transfected with miR-192 or transfected with a luciferase control, demonstrating that miR-192 induces a G1 arrest phenotype, as described in Example 6;

FIG. 6B graphically illustrates the results of cell cycle analysis of transfected HCT116DICER^{ex5} cells after treatment with nocodazole, wherein the cells were either transfected with 0.1 nM of a pool of siRNAs targeting a G1 set of miR-192 responsive genes, or transfected with a luciferase control, demonstrating that the siRNA G1 pool at a concentration of 0.1 nM phenocopies the miR-192 G1 arrest phenotype as described in Example 6;

FIG. 6C graphically illustrates the results of cell cycle analysis of transfected HCT116DICER^{ex5} cells after treatment with nocodazole, wherein the cells were either transfected with 0.01 nM of a pool of siRNAs targeting a G1 set of miR-192 responsive genes or transfected with a luciferase control, demonstrating that the lower concentration of siRNA G1 pool does not result in a miR-192 G1 arrest phenotype as described in Example 6;

FIG. 7A graphically illustrates the results of cell cycle analysis of transfected HCT116DICER^{ex5} cells after treatment with aphidicolin, wherein the cells were either transfected with miR-192 or transfected with a luciferase control, demonstrating that miR-192 induces a G2 arrest phenotype, as described in Example 6;

FIG. 7B graphically illustrates the results of cell cycle analysis of transfected HCT116DICER^{ex5} cells after treatment with aphidicolin, wherein the cells were either transfected with 0.1 nM of a pool of siRNAs targeting a G2 set of miR-192 responsive genes, or transfected with a luciferase control, demonstrating that the siRNA G2 pool at a concentration of 0.1 nM phenocopies the miR-192 G2 arrest phenotype as described in Example 6;

FIG. 7C graphically illustrates the results of cell cycle analysis of transfected HCT116DICER^{ex5} cells after treatment with aphidicolin, wherein the cells were either transfected with 0.01 nM of a pool of siRNAs targeting a G2 set of miR-192 responsive genes or transfected with a luciferase control, demonstrating that the lower concentration of siRNA G2 pool does not result in a miR-192 G2 arrest phenotype as described in Example 6;

FIG. 8A is a diagram of the canonical G1-S cell cycle checkpoint network, illustrating the members of the network found to be regulated by miR-192/miR-215 by microarray analysis (shown as black ovals) and the members of the network that were confirmed to be direct miR-192/miR-215 targets (shown as hatched ovals), as described in Example 6; and

FIG. 8B is a diagram of the canonical G2-M cell cycle checkpoint network, illustrating the members of the network found to be regulated by miR-192/miR-215 by microarray analysis (shown as black ovals) and the members of the network that were confirmed to be direct miR-192/miR-215 targets (shown as hatched ovals) as described in Example 6.

### DETAILED DESCRIPTION

This section presents a detailed description of the many different aspects and embodiments that are representative of the inventions disclosed herein. This description is by way of several exemplary illustrations of varying detail and specificity. Other features and advantages of these embodiments are apparent from the additional descriptions provided herein, including the different examples. The provided examples illustrate different components and methodology useful in practicing various embodiments of the invention. The examples are not intended to limit the claimed invention. Based on the present disclosure, the ordinary skilled artisan can identify and employ other components and methodology useful for practicing the present invention.

I. Definitions

Unless defined otherwise, all technical and scientific terms used herein have the meaning commonly understood by one of ordinary skill in the art to which this invention belongs. Practitioners are particularly directed to Sambrook et al., Molecular Cloning: A Laboratory Manual, 2d ed., Cold Spring Harbor Press, Plainsview, New York (1989); and Ausubel et al., Current Protocols in Molecular Biology (Supplement 47), John Wiley & Sons, New York (1999), for definitions and terms of the art.

It is contemplated that the use of the term “about” in the context of the present invention is to connote inherent problems with precise measurement of a specific element, characteristic, or other trait. Thus, the term “about,” as used herein in the context of the claimed invention, simply refers to an amount or measurement that takes into account single or collective calibration and other standardized errors generally associated with determining that amount or measurement. For example, a concentration of “about” 100 mM of Tris can encompass an amount of 100 mM±0.5 mM, if 0.5 mM represents the collective error bars in arriving at that concentration. Thus, any measurement or amount referred to in this application can be used with the term “about” if that measurement or amount is susceptible to errors associated with calibration or measuring equipment, such as a scale, pipetteman, pipette, graduated cylinder, etc.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only, or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “approximately” or “about” in reference to a number are generally taken to include numbers that fall within a range of 5% in either direction (greater than or less than) of the number unless otherwise stated or otherwise evident from the context (except where such number would exceed 100% of a possible value). Where ranges are stated, the endpoints are included within the range unless otherwise stated or otherwise evident from the context.

It is contemplated that any embodiment discussed in this specification can be implemented with respect to any method, kit, reagent, or composition of the invention, and vice versa. Furthermore, compositions of the invention can be used to achieve methods of the invention.

As used herein, the term “gene” has its meaning as understood in the art. However, it will be appreciated by those of ordinary skill in the art that the term “gene” may include gene regulatory sequences (e.g., promoters, enhancers, etc.) and/or intron sequences. It will further be appreciated that definitions of “gene” include references to nucleic acids that do not encode proteins but rather encode functional RNA molecules such as tRNAs. For clarity, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. In some cases, the gene includes regulatory sequences involved in transcription, or message production or composition. In other embodiments, the gene comprises transcribed sequences that encode for a protein, polypeptide or peptide. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof.

In particular embodiments, the transcribed nucleotide sequence comprises at least one functional protein, polypeptide and/or peptide encoding unit. As will be understood by those in the art, this functional term “gene” includes both genomic sequences, RNA or cDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

As used herein, the term “microRNA species”, “microRNA”, “miRNA”, or “mi-R” refers to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15 to 25, or 20 to 30 nucleotides in length, and more often between about 16 to 24, 17 to 23, 18 to 22, 19 to 21, or 21 to 24 nucleotides in length. MicroRNAs include processed sequences as well as corresponding long primary transcripts (pri-miRNAs) and processed precursors (pre-miRNAs). Some microRNA molecules function in living cells to regulate gene expression via RNA interference. A representative set of microRNA species is described in the publicly available miRBase sequence database as described in Griffith-Jones et al., Nucleic Acids Research 32:D109-D111 (2004) and Griffith-Jones et al., Nucleic Acids Research 34:D140-D144 (2006), accessible on the World Wide Web at the Wellcome Trust Sanger Institute website.

As used herein, the term “microRNA family” refers to a group of microRNA species that share identity across at least 6 consecutive nucleotides within nucleotide positions 1 to 12 of the 5′ end of the microRNA molecule, also referred to as the “seed region”, as described in Brennecke, J., et al., PloS biol. 3(3):pe85 (2005).

Families of microRNAs have been identified whose members share a region of 5′ identity but differ in their 3′ ends. It has been shown that two different microRNA family members that shared a common 5′ sequence that was complementary to a single 8-mer seed site in the bagpipe 3′ UTR were capable of repressing expression of a reporter gene containing the 8-mer target, even though the 3′ ends of the microRNAs differed, indicating that the target site was responsive to both microRNAs in this family (Brennecke et al., PloS Biology 3(3):e85 (2005)).

As used herein, the term “microRNA family member” refers to a microRNA species that is a member of a microRNA family.

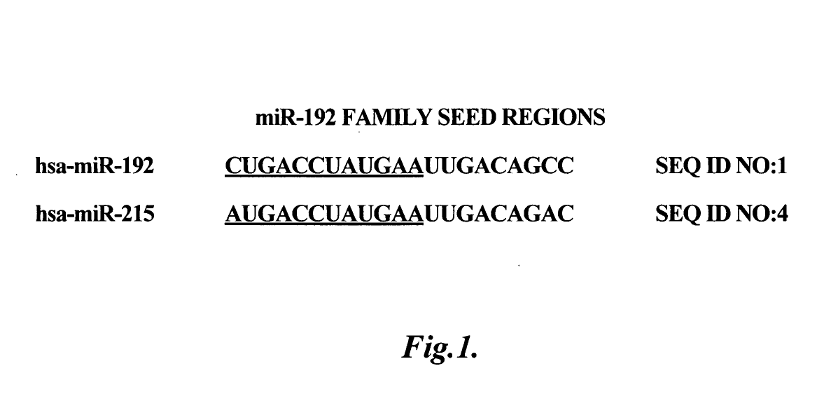

As used herein “miR-192 family” refers to miR-192 and miR-215. FIG. 1 provides an alignment of microRNA sequences for the miR-192 family members, with conserved seed regions underlined. As demonstrated in more detail in EXAMPLES 1-6, it has been found that members of the miR-192 family regulate cell cycle transition.

As used herein, “miR-192” refers to SEQ ID NO:1 (5′ CUGACCUAUGAAUUGACAGCC 3′) and precursor RNAs sequences thereof, an example of which is SEQ ID NO:2. (5′ GCCGAGACCGAGUGCACAGGGCUCUGACCUAUGAAUUGACAGCCAGUGCUCUCGUCUCCCC UCUGGCUGCCAAUUCCAUAGGUCACAGGUAUGUUCGCCUCAAUGCCAGC-3′)

As used herein, “miR-192 seed region” refers to SEQ ID NO:3 (5′ CUGACCUAUGAA-3′).

As used herein, “miR-215” refers to SEQ ID NO:4 (5′ AUGACCUAUGAAUUGACAGAC 3′) and precursor RNAs sequences thereof, an example of which is SEQ ID NO:5 (5′AUCAUUCAGAAAUGGUAUACAGGAAAAUGACCUAUGAAUUGACAGACAAUAUAGCUGAG UUUGUCUGUCAUUUCUUUAGGCCAAUAUUCUGUAUGACUGUGCUACUUCAA 3′)

As used herein, “miR-215 seed region” refers to SEQ ID NO:6 (5′ AUGACCUAUGAA3).

As used herein, the term “RNA interference” or “RNAi” refers to the silencing or decreasing of gene expression by iRNA agents (e.g., siRNAs, miRNAs, shRNAs), via the process of sequence-specific, post-transcriptional gene silencing in animals and plants, initiated by an iRNA agent that has a seed region sequence in the iRNA guide strand that is complementary to a sequence of the silenced gene.

As used herein, the term “siNA agent” (abbreviation for “small interfering nucleic acid agent”), refers to a nucleic acid agent, for example RNA, or chemically modified RNA, which can down-regulate the expression of a target gene. While not wishing to be bound by theory, an siNA agent may act by one or more of a number of mechanisms, including post-transcriptional cleavage of a target mRNA, or pre-transcriptional or pre-translational mechanisms. An siNA agent can include a single strand (ss) or can include more than one strands, e.g., it can be a double stranded (ds) siNA agent.

As used herein, the term “single strand siRNA agent” or “ssRNA” is an iRNA agent which consists of a single molecule. It may include a duplexed region, formed by intra-strand pairing, e.g., it may be, or include, a hairpin or panhandle structure. The ssRNA agents of the present invention include transcripts that adopt stem-loop structures, such as shRNA, that are processed into a double stranded siRNA.

As used herein, the term “ds siNA agent” is a dsNA (double stranded nucleic acid (NA)) agent that includes two strands that are not covalently linked, in which interchain hybridization can form a region of duplex structure. The dsNA agents of the present invention include silencing dsNA molecules that are sufficiently short that they do not trigger the interferon response in mammalian cells.

As used herein, the term “siRNA” refers to a small interfering RNA. siRNA include short interfering RNA of about 15-60, 15-50, 15-50, or 15-40 (duplex) nucleotides in length, more typically about 15-30, 15-25, or 19-25 (duplex) nucleotides in length, and preferably about 20-24 or about 21-22 or 21-23 (duplex) nucleotides in length (e.g., each complementary sequence of the double stranded siRNA is 15-60, 15-50, 15-50, 15-40, 15-30, 15-25, or 19-25 nucleotides in length, preferably about 20-24 or about 21-22 or 21-23 nucleotides in length, preferably 19-21 nucleotides in length, and the double stranded siRNA is about 15-60, 15-50, 15-50, 15-40, 15-30, 15-25, or 19-25 base pairs in length, preferably about 20-24 or about 21-22 or 19-21 or 21-23 base pairs in length). siRNA duplexes may comprise 3′ overhangs of about 1 to about 4 nucleotides, preferably of about 2 to about 3 nucleotides and 5′ phosphate termini. In some embodiments, the siRNA lacks a terminal phosphate.

Non limiting examples of siRNA molecules of the invention may include a double-stranded polynucleotide molecule comprising self-complementary sense and antisense regions, wherein the antisense region comprises nucleotide sequence that is complementary to nucleotide sequence in a target nucleic acid molecule or a portion thereof (alternatively referred to as the guide region, or guide strand when the molecule contains two separate strands) and the sense region having nucleotide sequence corresponding to the target nucleic acid sequence or a portion thereof (also referred as the passenger region, or the passenger strand, when the molecule contains two separate strands). The siRNA can be assembled from two separate oligonucleotides, where one strand is the sense strand and the other is the antisense strand, wherein the antisense and sense strands are self-complementary (i.e., each strand comprises a nucleotide sequence that is complementary to the nucleotide sequence in the other strand; such as where the antisense strand and sense strand form a duplex or double stranded structure, for example wherein the double stranded region is about 18 to about 30, e.g., about 18, 19, 20, 21, 22, 23, 24, 25, 26, 27, 28, 29, or 30 base pairs); the antisense strand (guide strand) comprises nucleotide sequence that is complementary to nucleotide sequence in a target nucleic acid molecule or a portion thereof and the sense strand (passenger strand) comprises nucleotide sequence corresponding to the target nucleic acid sequence or a portion thereof (e.g., about 15 to about 25 nucleotides of the siRNA molecule are complementary to the target nucleic acid or a portion thereof). Typically, a short interfering RNA (siRNA) refers to a double-stranded RNA molecule of about 17 to about 29 base pairs in length, preferably from 19-21 base pairs, one strand of which is complementary to a target mRNA, that when added to a cell having the target mRNA, or produced in the cell in vivo, causes degradation of the target mRNA. Preferably, the siRNA is perfectly complementary to the target mRNA. But it may have one or two mismatched base pairs.

Alternatively, the siRNA is assembled from a single oligonucleotide, where the self-complementary sense and antisense regions of the siRNA are linked by means of a nucleic acid based or non-nucleic acid-based linker(s). The siRNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof, and the sense region having nucleotide sequence corresponding to the target nucleic acid sequence or a portion thereof. The siRNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siRNA molecule capable of mediating RNAi. The siRNA can also comprise a single stranded polynucleotide having nucleotide sequence complementary to nucleotide sequence in a target nucleic acid molecule or a portion thereof (for example, where such siRNA molecule does not require the presence within the siRNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell 110:563-574; and Schwarz et al., 2002, Molecular Cell, 10:537-568), or 5′,3′-diphosphate. In certain embodiments, the siRNA molecule of the invention comprises separate sense and antisense sequences or regions, wherein the sense and antisense regions are covalently linked by nucleotide or non-nucleotide linker molecules as are known in the art, or are alternately non-covalently linked by ionic interactions, hydrogen bonding, van der waals interactions, hydrophobic interactions, and/or stacking interactions. In certain embodiments, the siRNA molecules of the invention comprise nucleotide sequence that is complementary to nucleotide sequence of a target gene. In another embodiment, the siRNA molecule of the invention interacts with the nucleotide sequence of a target gene in a manner that causes inhibition of expression of the target gene.

As used herein, the siRNA molecules need not be limited to those molecules containing only RNA, but may further encompass chemically-modified nucleotides and non-nucleotides. International Publication Nos. WO 2005/078097, WO 2005/0020521, and WO2003/070918 detail various chemical modifications to RNAi molecules, wherein the contents of each reference are hereby incorporated by reference in their entirety. In certain embodiments, for example, the short interfering nucleic acid molecules may lack 2′-hydroxy (2′-OH) containing nucleotides. The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., 2002 PNAS USA 99:9942-7; Calegari et al., 2002, PNAS USA 99:14236; Byrom et al., 2003, Ambion TechNotes 10(1):4-6; Kawasaki et al., 2003, Nucleic Acids Res. 31:981-7; Knight and Bass, 2001, Science 293:2269-71; and Robertson et al., 1968, J. Biol. Chem. 243:82). The long dsRNA can encode for an entire gene transcript or a partial gene transcript.

As used herein, “percent modification” refers to the number of nucleotides in each strand of the siRNA, or in the collective dsRNA, that have been modified. Thus 19% modification of the antisense strand refers to the modification of up to 4 nucleotides/bp in a 21 nucleotide sequence (21 mer). 100% refers to a fully modified dsRNA. The extent of chemical modification will depend upon various factors well known to one skilled in the art. Such as, for example, target mRNA, off-target silencing, degree of endonuclease degradation, etc.

As used herein, the term “shRNA” or “short hairpin RNAs” refers to an RNA molecule that forms a stem-loop structure in physiological conditions, with a double-stranded stem of about 17 to about 29 base pairs in length, wherein one strand of the base-paired stem is complementary to the mRNA of a target gene. The loop of the shRNA stem-loop structure may be any suitable length that allows inactivation of the target gene in vivo. While the loop may be from 3 to 30 nucleotides in length, typically it is 1-10 nucleotides in length. The base paired stem may be perfectly base paired or may have 1 or 2 mismatched base pairs. The duplex portion may, but typically does not, contain one or more bulges consisting of one or more unpaired nucleotides. The shRNA may have non-base-paired 5′ and 3′ sequences extending from the base-paired stem. Typically, however, there is no 5′ extension. The first nucleotide of the shRNA at the 5′ end is a G, because this is the first nucleotide transcribed by polymerase III. If G is not present as the first base in the target sequence, a G may be added before the specific target sequence. The 5′ G typically forms a portion of the base-paired stem. Typically, the 3′ end of the shRNA is a poly U segment that is a transcription termination signal and does not form a base-paired structure. As described in the application and known to one skilled in the art, shRNAs are processed into siRNAs by the conserved cellular RNAi machinery. Thus shRNAs are precursors of siRNAs and are, in general, similarly capable of inhibiting expression of a target mRNA transcript. For the purpose of description, in certain embodiments, the shRNA constructs of the invention target one or more mRNAs that are targeted by miR-34a, miR-34b, miR-34c or miR-449. The strand of the shRNA that is antisense to the target gene transcript is also known as the “guide strand”.

As used herein, the term “microRNA responsive target site” refers to a nucleic acid sequence ranging in size from about 5 to about 25 nucleotides (such as 5, 6, 7, 8, 9, 10, 11, 12, 13, 14, 15, 16, 17, 18, 19, 20, 21, 22, 23, 24, or 25 nucleotides) that is complementary, or essentially complementary, to at least a portion of a microRNA molecule. In some embodiments, the microRNA responsive target site comprises at least 6 consecutive nucleotides, at least 7 consecutive nucleotides, at least 8 consecutive nucleotides, or at least 9 nucleotides that are complementary to the seed region of a microRNA molecule (i.e., within nucleotide positions 1 to 12 of the 5′ end of the microRNA molecule, referred to as the “seed region”. In some embodiments, the miR-192 responsive site comprises at least one copy (or multiple copies) of SEQ ID NO:379 located in the 3′ UTR of a gene.

The phrase “inhibiting expression of a target gene” refers to the ability of an RNAi agent, such as an siRNA, to silence, reduce, or inhibit expression of a target gene. Said another way, to “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the RNAi agent. For example, an embodiment of the invention proposes inhibiting, down-regulating, or reducing expression of one or more miR-192 responsive genes, by introduction of an miR-192-like siRNA molecule, below the level observed for that miR-192 responsive genes in a control cell to which an miR-192-like siRNA molecule has not been introduced. In another embodiment, inhibition, down-regulation, or reduction contemplates inhibition of the target mRNA below the level observed in the presence of, for example, an siRNA molecule with scrambled sequences or with mismatches. In yet another embodiment, inhibition, down-regulation, or reduction of gene expression with a siRNA molecule of the instant invention is greater in the presence of the invention siRNA, e.g., siRNA that down-regulates one or more miR-192 responsive gene mRNA levels, than in its absence. In one embodiment, inhibition, down-regulation, or reduction of gene expression is associated with post transcriptional silencing, such as RNAi mediated cleavage of a target nucleic acid molecule (e.g., RNA) or inhibition of translation.

To examine the extent of gene silencing, a test sample (e.g., a biological sample from an organism of interest expressing the target gene(s) or a sample of cells in culture expressing the target gene(s)) is contacted with an siRNA that silences, reduces, or inhibits expression of the target gene(s). Expression of the target gene in the test sample is compared to expression of the target gene in a control sample (e.g., a biological sample from an organism of interest expressing the target gene or a sample of cells in culture expressing the target gene) that is not contacted with the siRNA. Control samples (i.e., samples expressing the target gene) are assigned a value of 100%. Silencing, inhibition, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, or 10%. Suitable assays include, e.g., examination of protein or mRNA levels using techniques known to those of skill in the art, such as dot blots, northern blots, in situ hybridization, ELISA, microarray hybridization, immunoprecipitation, enzyme function, as well as phenotypic assays known to those of skill in the art.

An “effective amount” or “therapeutically effective amount” of an siRNA or an RNAi agent is an amount sufficient to produce the desired effect, e.g., inhibition of expression of a target sequence in comparison to the normal expression level detected in the absence of the siRNA or RNAi agent Inhibition of expression of a target gene or target sequence by an siRNA or RNAi agent is achieved when the expression level of the target gene mRNA or protein is about 90%, 80%, 70%, 60%, 50%, 40%, 30%, 25%, 20%, 15%, 10%, 5%, or 0% relative to the expression level of the target gene mRNA or protein of a control sample.

As used herein, the term “isolated” in the context of an isolated nucleic acid molecule, is one which is altered or removed from the natural state through human intervention. For example, an RNA naturally present in a living animal is not “isolated.” A synthetic RNA or dsRNA or microRNA molecule that is partially or completely separated from the coexisting materials of its natural state, is “isolated.” Thus, an miRNA molecule which is deliberately delivered to or expressed in a cell is considered an “isolated” nucleic acid molecule.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit,” but the use of the word “modulate” is not limited to this definition.

As used herein, “RNA” refers to a molecule comprising at least one ribonucleotide residue. The term “ribonucleotide” means a nucleotide with a hydroxyl group at the 2′ position of a β-D-ribofuranose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an RNAi agent or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs of naturally-occurring RNA.

As used herein, the term “complementary” refers to nucleic acid sequences that are capable of base-pairing according to the standard Watson-Crick complementary rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA.

As used herein, the term “essentially complementary” with reference to microRNA target sequences refers to microRNA target nucleic acid sequences that are longer than 8 nucleotides that are complementary (an exact match) to at least 8 consecutive nucleotides of the 5′ portion of a microRNA molecule from nucleotide positions 1 to 12, (also referred to as the “seed region”), and are at least 65% complementary (such as at least 70%, at least 75%, at least 80%, at least 85%, at least 90%, at least 95%, or at least 96% identical) across the remainder of the microRNA target nucleic acid sequence as compared to a naturally occurring miR-192 family member. The comparison of sequences and determination of percent identity and similarity between two sequences can be accomplished using a mathematical algorithm of Karlin and Altschul (PNAS 87:2264-2268, 1990), modified as in Karlin and Altschul (PNAS 90:5873-5877, 1993). Such an algorithm is incorporated into the NBLAST and XBLAST programs of Altschul et al. (J. Mol. Biol. 215:403-410, 1990).

As used herein, the term “gene” encompasses the meaning known to one of skill in the art, i.e., a nucleic acid (e.g., DNA or RNA) sequence that comprises coding sequences necessary for the production of an RNA and/or a polypeptide, or its precursor, as well as noncoding sequences (untranslated regions) surrounding the 5′ and 3′ ends of the coding sequences. The term “gene” encompasses both cDNA and genomic forms of a gene. The term “gene” also encompasses nucleic acid sequences that comprise microRNAs and other non-protein encoding sequences, including, for example, transfer RNAs, ribosomal RNAs, etc. A functional polypeptide can be encoded by a full length coding sequence or by any portion of the coding sequence as long as the desired activity or functional properties (e.g., enzymatic activity, ligand binding, signal transduction, antigenic presentation) of the polypeptide are retained. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

The term “gene expression”, as used herein, refers to the process of transcription and translation of a gene to produce a gene product, be it RNA or protein. Thus, modulation of gene expression may occur at any one or more of many levels, including transcription, post-transcriptional processing, translation, post-translational modification, and the like.

As used herein, the term “expression cassette” refers to a nucleic acid molecule which comprises at least one nucleic acid sequence that is to be expressed, along with its transcription and translational control sequences. The expression cassette typically includes restriction sites engineered to be present at the 5′ and 3′ ends such that the cassette can be easily inserted, removed, or replaced in a gene delivery vector. Changing the cassette will cause the gene delivery vector into which it is incorporated to direct the expression of a different sequence.

As used herein, the term “phenotype” encompasses the meaning known to one of skill in the art, including modulation of the expression of one or more genes, as measured by gene expression analysis or protein expression analysis.

As used herein, the term “proliferative disease” or “cancer” refers to any disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia; AIDS related cancers such as Kaposi's sarcoma; breast cancers; bone cancers such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, schwannomas, and metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration, and other proliferative diseases and conditions such as restenosis and polycystic kidney disease, and any other cancer or proliferative disease, condition, trait, genotype, or phenotype that can respond to the modulation of disease-related gene expression in a cell or tissue, alone or in combination with other therapies.

As used herein, the term “source of biological knowledge” refers to information that describes the function (e.g., at molecular, cellular, and system levels), structure, pathological roles, toxicological implications, etc., of a multiplicity of genes. Various sources of biological knowledge can be used for the methods of the invention, including databases and information collected from public sources such as Locuslink, Unigene, SwissTrEMBL, etc., and organized into a relational database following the concept of the central dogma of molecular biology. In some embodiments, the annotation systems used by the Gene Ontology™ (GO) Consortium or similar systems are employed. GO is a dynamic controlled vocabulary for molecular biology which can be applied to all organisms. As knowledge of gene function is accumulating and changing, it is developed and maintained by the Gene Ontology™ Consortium (“Gene Ontology: tool for the unification of biology.” The Gene Ontology Consortium (2000), Nature Genet. 25:25-29).

As used herein, the term to “inhibit the proliferation of a mammalian cell” means to kill the cell, or permanently or temporarily arrest the growth of the cell. Inhibition of a mammalian cell can be inferred if the number of such cells, either in an in vitro culture vessel, or in a subject, remains constant or decreases after administration of the compositions of the invention. An inhibition of tumor cell proliferation can also be inferred if the absolute number of such cells increases, but the rate of tumor growth decreases.

As used herein, the terms “measuring expression levels,” “obtaining an expression level” and the like, include methods that quantify a gene expression level of, for example, a transcript of a gene, including microRNA (miRNA) or a protein encoded by a gene, as well as methods that determine whether a gene of interest is expressed at all. Thus, an assay which provides a “yes” or “no” result, without necessarily providing quantification of an amount of expression, is an assay that “measures expression” as that term is used herein. Alternatively, a measured or obtained expression level may be expressed as any quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see for example, U.S. Pat. No. 5,569,588) nuclease protection, RT-PCR, microarray profiling, differential display, 2D gel electrophoresis, SELDI-TOF, ICAT, enzyme assay, antibody assay, and the like.

As used herein, an “isolated nucleic acid” is a nucleic acid molecule that exists in a physical form that is non-identical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequences, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

The terms “over-expression”, “over-expresses”, “over-expressing”, and the like, refer to the state of altering a subject such that expression of one or more genes in said subject is significantly higher, as determined using one or more statistical tests, than the level of expression of said gene or genes in the same unaltered subject or an analogous unaltered subject.

As used herein, a “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, a chemically synthesized nucleic acid that has not been purified.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least 2-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

As used herein, “subject”, refers to an organism or to a cell sample, tissue sample or organ sample derived therefrom, including, for example, cultured cell lines, biopsy, blood sample, or fluid sample containing a cell. For example, an organism may be an animal, including but not limited to, an animal such as a cow, a pig, a mouse, a rat, a chicken, a cat, a dog, etc., and is usually a mammal, such as a human.

As used herein, “TP53 pathway” refers to proteins, and their corresponding genes, that function both upstream and downstream of TP53, including, for example, proteins that are involved in or required for perception of DNA damage, modulation of TP53 activity, cell cycle arrest, and apoptosis. TP53 pathway includes, but is not limited to, the genes, and proteins encoded thereby, listed in Table 1 (see also Vogelstein, et al., 2000, Nature 408:307-310; Woods and Vousden, 2001, Experimental Cell Research 264:56-66; El-Deiry, 1998, Semin. Cancer Biology 8:345-357; and Prives and Hall, 1999, J. Pathol. 1999 187:112-126).

II. Aspects and Embodiments of the Invention

In accordance with the foregoing, in one aspect the invention provides a method of inhibiting proliferation of a mammalian cell comprising introducing into the mammalian cell an effective amount of at least one small interfering nucleic acid (siNA) agent that inhibits the level of expression of at least one miR-192 responsive gene selected from TABLE 3.

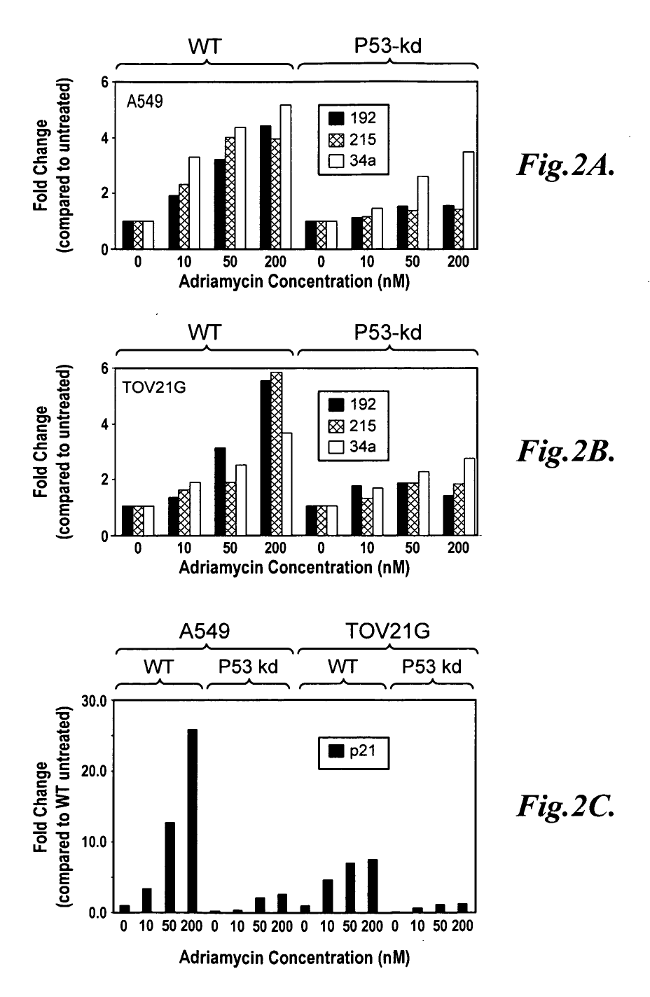

As demonstrated in Example 1 and FIG. 2, it has been determined that genotoxic stress promotes p53-dependent up-regulation of the miR-192 family. As described in Example 2, using gene expression profiling and RNAi-mediated gene silencing, a set of downstream effectors of miR-192/miR-215 was identified that include a number of key regulators of DNA synthesis and the G₁ and G₂ cell cycle checkpoints. It has been further determined that enforced expression of miR-192 or miR-215 leads to G1 and G2 cell cycle arrest, as described in Example 3. As shown in Examples 4-6, transfection of cells with siRNA pools directed to miR-192/miR-215 responsive targets is effective to phenocopy the cell cycle arrest phenotype of miR-192/miR-215.

In accordance with the foregoing, in one aspect, the present invention provides therapeutic miR-192, miR-215, and duplex mimetics functionally and structurally related to miR-192 and miR-215, as well as siRNA or shRNA compositions are provided that may be used in the methods of inhibiting proliferation of mammalian cells.

The methods of this aspect of the invention may be practiced using any cell type, such as primary cells, or an established line of cultured cells may be used in the practice of the methods of the invention. For example, the methods may be used in any mammalian cell from a variety of species, such as a cow, horse, mouse, rat, dog, pig, goat, or primate, including a human. In some embodiments, the methods may be used in a mammalian cell type that has been modified, such as a cell type derived from a transgenic animal or a knockout mouse.

In some embodiments, the method of the invention is practiced using a cancer cell type. Representative examples of suitable cancer cell types that can be cultured in vitro and used in the practice of the present invention are colon cancer cells, such as wild type HCT116, wild-type DLD-1, HCT116-Dicer^{ex5} and DLD-1 Dicer^{ex5} cells described in Cummins, J. M., et al., PNAS 103(10):3687-3692 (2006), osteosarcoma cells, liver cancer cells, melanoma cancer cells, and head and neck squamous cell carcinoma cells. Other non-limiting examples of suitable cancer cell types include A549, MCF7, and TOV21G and are available from the American Type Culture Collection, Rockville, Md. In further embodiments, the cell type is a miRNA-192 or miR-215 mediated cancer cell type.

For example, microarray analyses of colon adenocarcinomas found that miR-192/miR-215 expression is significantly reduced in tumor samples relative to matched adjacent non-involved tissue (Schetter, A. J., et al., JAMA 299:425-436 (2008)). Interestingly, several of the transcripts identified in TABLE 3 as miR-192/miR-215 targets have been reported as being over-expressed in tumors, including DTL over-expression in aggressive liver cancer (Pan, H. W., et al., Cell Cycle 5:2676-2687 (2006)), and CDC7 up-regulation in endocrine tumors, thyroid tumors, melanomas, and head and neck squamous cell carcinomas (Mould, A. W., et al., Int. J. Cancer 121:776-783 (2007); Slebos, R. J., et al., Clin. Cancer Res. 12:701-709 (2006); Kaufman, W. K., et al., J. Invest. Dermatol. 128:175-187 (2008); Fluge, O., et al., Thyroid 16:161-175 (2006)).

One embodiment of the method involves use of a therapeutically sufficient amount of a composition comprising an siNA agent comprising a miR-192 family member selected from synthetic duplex mimetics of miR-192 or miR-215, to inhibit mammalian cell proliferation. Therapeutic synthetic duplex mimetics of miR-192, or miR-215 comprise a guide strand contiguous nucleotide sequence of at least 18 nucleotides, wherein said guide strand comprises a seed region consisting of nucleotide positions 1 to 12, wherein position 1 represents the 5′ end of said guide strand and wherein said seed region comprises a nucleotide sequence of at least six contiguous nucleotides that is identical to six contiguous nucleotides within a sequence selected from the group consisting of SEQ ID NO:3, or SEQ ID NO:6. In certain embodiments, at least one of the two strands further comprises a 1-4 nucleotide, preferably a 2 nucleotide, 3′ overhang. The nucleotide overhang can include any combination of a thymine, uracil, adenine, guanine, or cytosine, or derivatives or analogues thereof. The nucleotide overhang in certain aspects is a 2 nucleotide overhang, where both nucleotides are thymine. Importantly, when the dsRNA comprising the sense and antisense strands is administered, it directs target specific interference and bypasses an interferon response pathway.

In one embodiment, the present invention provides a synthetic duplex microRNA mimetic comprising (i) a guide strand nucleic acid molecule consisting of a nucleotide sequence of 18 to 25 nucleotides, said guide strand nucleotide sequence comprising a seed region nucleotide sequence and a non-seed region nucleotide sequence, said seed region consisting essentially of nucleotide positions 1 to 12 and said non-seed region consisting essentially of nucleotide positions 13 to the 3′ end of said guide strand, wherein position 1 of said guide strand represents the 5′ end of said guide strand, wherein said seed region further comprises a consecutive nucleotide sequence of at least 6 nucleotides that is identical in sequence to a nucleotide sequence selected from the group consisting of SEQ ID NO:3 and SEQ ID NO:6; and (ii) a passenger strand nucleic acid molecule consisting of a nucleotide sequence of 18 to 25 nucleotides, said passenger strand comprising a nucleotide sequence that has at least one nucleotide sequence difference compared with the true reverse complement sequence of the seed region of the guide strand, wherein the at least one nucleotide difference is located within nucleotide position 13 to the 3′ end of said passenger strand. In one embodiment of this aspect of the invention, the guide strand of the synthetic duplex microRNA mimetic is selected from the group consisting of miR-192 (SEQ ID NO:1) and miR-215 (SEQ ID NO:4). In one embodiment, the passenger strand of the synthetic duplex microRNA mimetic is selected from the group consisting of SEQ ID NO:7 and SEQ ID NO:10.

In order to enhance the stability of the short interfering nucleic acids, the 3′ overhangs can also be stabilized against degradation. In one embodiment, the 3′ overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

As used herein, a “3′ overhang” refers to at least one unpaired nucleotide extending from the 3′ end of an siRNA sequence. The 3′ overhang can include ribonucleotides or deoxyribonucleotides or modified ribonucleotides or modified deoxyribonucleotides. The 3′ overhang is preferably from 1 to about 5 nucleotides in length, more preferably from 1 to about 4 nucleotides in length and most preferably from about 2 to about 4 nucleotides in length. The 3′ overhang can occur on the sense or antisense sequence, or on both sequences, of an RNAi construct. The length of the overhangs can be the same or different for each strand of the duplex. Most preferably, a 3′ overhang is present on both strands of the duplex, and the overhang for each strand is 2 nucleotides in length. For example, each strand of the duplex can comprise 3′ overhangs of dithymidylic acid (“tt”) or diuridylic acid (“uu”).

Another aspect of the invention provides chemically modified siRNA constructs. For example, the siRNA agent can include a non-nucleotide moiety. A chemical modification or other non-nucleotide moiety can stabilize the sense (guide strand) and antisense (passenger strand) sequences against nucleolytic degradation. Additionally, conjugates can be used to increase uptake and target uptake of the siRNA agent to particular cell types. Thus, in one embodiment, the siRNA agent includes a duplex molecule wherein one or more sequences of the duplex molecule is chemically modified. Non-limiting examples of such chemical modifications include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5′-C-methyl nucleotides, and terminal glyceryl and/or inverted deoxy abasic residue incorporation. These chemical modifications, when used in siRNA agents, can help to preserve RNAi activity of the agents in cells and can increase the serum stability of the siRNA agents.

In one embodiment, the first, and optionally or preferably the first two, internucleotide linkages at the 5′ end of the antisense and/or sense sequences are modified, preferably by a phosphorothioate. In another embodiment, the first, and perhaps the first two, three, or four, internucleotide linkages at the 3′ end of a sense and/or antisense sequence are modified, for example, by a phosphorothioate. In another embodiment, the 5′ end of both the sense and antisense sequences, and the 3′ end of both the sense and antisense sequences are modified as described.

In some embodiments of the invention, the siNA agent comprises gene-specific agents designed to inhibit a miR-192/miR-215 responsive gene of interest, including RNA inhibitors such as antisense oligonucleotides, iRNA agents, and protein inhibitors, such as antibodies, soluble receptors, and the like. iRNA agents encompass any RNA agent which can downregulate the expression of a target gene, including siRNA molecules and shRNA molecules. The siRNA molecules may be designed to inhibit a particular target gene by using an algorithm developed to increase efficiency of the siRNAs for silencing while minimizing their off-target effects, as described in Jackson et al., Nat. Biotech. 21:635-637 (2003), International Publication Nos. WO 2006/006948 and WO 2005/042708, incorporated herein by reference. Exemplary siRNA sequences designed to target miR-192/miR-215 down-regulated transcripts are provided below in TABLE 5.

The microRNA, and iRNA agents (including shRNA, and siRNA molecules) for use in the practice of the methods of the invention and to produce the compositions of the invention may be chemically synthesized or recombinantly produced using methods known in the art. for example, the RNA products may be chemically synthesized using appropriately protected ribonucleoside phosphoramidites and a conventional DNA/RNA synthesizer. Commercial suppliers of synthetic RNA molecules or synthesis reagents include Proligo (Hamburg Germany) and Dharmacon Research (Lafayette, Colo.). Exemplary microRNA molecules that may be used to practice various embodiments of the methods of this aspect of the invention are provided in TABLE 1.

Alternatively, microRNA gene products and iRNA agents can be expressed from recombinant circular or linear DNA plasmids using any suitable promoter. Suitable promoters for expressing RNA from a plasmid include the U6 or H1 RNA PolIII promoter sequences, or the cytomegalovirus promoters. Selection of other suitable promoters for expressing RNA from a plasmid is within the skill in the art. The recombinant plasmids of the invention can also comprise inducible or regulatable promoters for expression of the microRNA or iRNA agent gene products in a desired cell type. For example, a vector may be designed to drive expression (e.g., using the PolIII promoter) of both the sense and antisense strands separately, which hybridize in vivo to generate siRNA.

In one embodiment, the iRNA agent is an shRNA. A vector may be used to drive expression of short hairpin RNA (shRNA), which are individual transcripts that adopt stem-loop structures, which are processed into siRNAs by the RNAi machinery in the cell. Typically, the shRNA design comprises two inverted repeats containing the sense and antisense target sequence separated by a loop sequence. Typically, the loop sequence contains 8 to 9 bases. A terminator sequence consisting of 5-6 polydTs is present at the 3′ end and one or more cloning sequences may be added to the 5′ end using complementary oligonucleotides. A website is available for design of such vectors, see, http://www.genelink.com/sirna/shRNAhelp.asp.

An shRNA vector may be designed with an inducible promoter. For example, a lentiviral vector may be used expressing tTS (tetracycline-controlled transcriptional repressor, Clontech). For example, a tetracycline-inducible shRNA designed to target a gene, such as PLK1 may be driven from an H1 promoter, as described in Jackson et al., RNA 12:1-9 (2006). The cells of interest are infected with recombinant lentivirus and shRNA expression is induced by incubation of the cells in the presence of 50 ng/mL of doxycycline.

In some embodiments, the present invention provides a method of inhibiting proliferation of a mammalian cell comprising introducing an effective amount of at least one gene-specific inhibitor of expression of at least one miR-192/miR-215 responsive gene selected from TABLE 3 into the mammalian cell. In some embodiments, the method comprises introducing an effective amount of at least one gene-specific inhibitor of expression of at least two miR-192/miR-215 responsive genes selected from TABLE 3 into the mammalian cell. In some embodiments, the method comprises introducing an effective amount of at least one gene-specific inhibitor of at least one miR-192/miR-215 responsive gene selected from TABLE 7 or TABLE 8 into the mammalian cell.

In some embodiments, a miR-192 responsive gene comprises at least one copy (or multiple copies) of SEQ ID NO:379 located in its 3′ UTR.

In some embodiments, the at least one miR-192/miR-215 responsive gene is selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

In some embodiments, the method comprises introducing a composition comprising an effective amount of a combination of nucleic acid molecules that inhibit at least two or more miR-192/miR-215 responsive targets selected from the group consisting of SEPT 10, LMNB2, HRH1, HOXA10, ERCC3, MIS12, MPHOSPHI1, CDC7, SMARCB1, and MAD2L1.

In some embodiments, the method comprises introducing a composition comprising an effective amount of a combination of nucleic acid molecules that inhibit at least two or more miR-192/miR-215 responsive targets selected from the group consisting of SMARCB1, MAD2L1, DTL, RACGAP1, MCM10, PIM1, DLG5, BCL2, CUL5, and PRPF38A.

As demonstrated in EXAMPLES 3-6, the methods of this aspect of the invention may be used to inhibit proliferation of a cancer cell.

In some embodiments, the gene-specific agents that inhibit at least one miR-192/miR-215 responsive target comprise iRNA agents, including siRNA molecules and shRNA molecules. Exemplary siRNA molecules useful in the practice of the method of the invention are provided in TABLE 5, TABLE 9, and TABLE 10. In some embodiments, the siRNA molecules comprise at least one dsRNA molecule comprising one nucleotide strand that is substantially identical to a portion of the mRNA encoding a gene listed in TABLE 3, such as, for example, SEPT 10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

In one particular embodiment, the gene-specific agent directed against at least one miR-192/miR-215 responsive gene is at least one dsRNA molecule comprising a double-stranded region, wherein one strand of the double-stranded region is substantially identical to 15 to 25 consecutive nucleotides of an mRNA encoding a gene set forth in TABLE 3 (such as, for example SEPT 10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A), and the second strand is substantially complementary to the first, and wherein at least one end of the dsRNA has an overhang of 1 to 4 nucleotides.

In one embodiment, the gene-specific agent comprises at least one dsRNA molecule comprising at least one of SEQ ID NO:13 to SEQ ID NO:120.

In some embodiments, the method comprises contacting cancer cells with a plurality of pools of siRNA molecules directed against at least two (such as at least three, at least four, at least five, at least six, at least seven, at least eight, at least nine, or all ten) of the miR-192/miR-215 responsive targets set forth in TABLE 9 or TABLE 10.

The siRNAs useful in the methods of the invention may be chemically synthesized and annealed before delivery to a cell or mammalian subject, as described supra. In some embodiments, the siRNAs are synthesized in vivo, such as from a plasmid expression system (see, e.g., Tuschl and Borkhardt, Molec. Interventions 2:158-167 (2002)). Exemplary constructs for making dsRNAs are described, for example, in U.S. Pat. No. 6,573,099. In some embodiments, the siRNA or shRNA inhibitory molecules inhibit expression of a target gene by at least 30%, such as 50%, such as 60%, such as 80%, or such as 90% up to 100%.

The siRNA and shRNA molecules can be delivered into cells in culture using electroporation or lipophilic reagents. The siRNA molecules can be delivered into a mammalian subject, for example, by intravenous injection, direct injection into a target site (e.g., into tumors), or into mice or rats by high-pressure tail-vein injection. It has been demonstrated that synthetic siRNAs can silence target gene expression in mammalian models. For example, McCaffrey et al. (Nature 418:38-39 (2002)) described silencing of a reporter gene in mice when the reporter gene and siRNA were injected simultaneously by high-pressure tail vein injections. Moreover, Soutsched et al. (Nature 432:173-178 (2004)) demonstrated that a synthetic siRNA downregulated expression of an endogenous target gene following intravenous injection in mice. Similarly, Pulukuir et al. (J. Biol. Chem 280:36529-36540 (2005)) demonstrated that injection of plasmids expressing short hairpin RNAs (shRNAs) into tumors in mice downregulated expression of the target gene in the tumors and also caused a decrease in tumor weight.

In one embodiment, the present invention provides compositions comprising a combination of nucleic acid molecules that are useful as inhibitors of at least two or more miR-192/miR-215 responsive targets selected from TABLE 3, TABLE 9, or TABLE 10. In some embodiments, the compositions comprise a combination of nucleic acid molecules that are useful as inhibitors of at least two or more miR-192/miR-215 responsive targets selected from the group consisting of SEPT 10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

In some embodiments, the compositions comprise a combination of nucleic acid molecules that are useful as inhibitors of at least two or more coordinately regulated miR-192/miR-215 responsive targets selected from the group consisting of SEPT 10, LMNB2, HRH1, HOXA10, ERCC3, MIS12, MPHOSPHI1, CDC7, SMARCB1, and MAD2L1.

In some embodiments, the compositions comprise a combination of nucleic acid molecules that are useful as inhibitors of at least two or more coordinately regulated miR-192/miR-215 responsive targets selected from the group consisting of SMARCB1, MAD2L1, DTL, RACGAP1, MCM10, PIM1, DLG5, BCL2, CUL5, and PRPF38A.

In some embodiments, the compositions comprise a nucleic acid molecule comprising a nucleic acid sequence of at least one of SEQ ID NO:13 to SEQ ID NO:120. The compositions according to this aspect of the invention are useful in the methods of the invention described herein.

In another aspect, the present invention provides an isolated dsRNA molecule comprising one nucleotide strand that is substantially identical to a sequence selected from the group consisting of SEQ ID NO:13 to SEQ ID NO:120. In some embodiments, the isolated dsRNA molecule comprises at least one of SEQ ID NO:13 to SEQ ID NO:120. In some embodiments, at least one strand of the isolated dsRNA molecule consists of at least one of SEQ ID NO:13 to SEQ ID NO:120. The isolated dsRNA molecules according to this aspect of the invention may be included in a composition for use in the methods of the invention.

In another embodiment, pharmaceutical compositions comprising nucleic acid molecules that inhibit at least one miR-192/miR-215 responsive target are provided. Such a composition contains from about 0.01 to 90% by weight (such as 1 to 20% or 1 to 10%) of a therapeutic agent of the invention in a pharmaceutically acceptable carrier. Solid formulations of the compositions for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Liquid formulations of the compositions for oral administration prepared in water or other aqueous vehicles may contain various suspending agents such as methylcellulose, alginate, tragacanth, pectin, kelgin, carageenan, acacia, polyvinylpyrrolidone, and polyvinyl alcohol.

Injectable formulations of the compositions may contain various carriers such as vegetable oils, dimethylacetamide, dimethylformamide, ethyl lactate, ethyl carbonate, isopropyl myristate, ethanol, or polyols (glycerol, propylene glycol, liquid polyethylene glycol and the like). For intravenous injections, water soluble versions of the compounds may be administered by the drip method, whereby a pharmaceutical formulation containing an antifungal agent and a physiologically acceptable excipient is infused. Physiologically acceptable excipients may include, for example, 5% dextrose, 0.9% saline, Ringer's solution, or other suitable excipients. Intramuscular preparations, e.g., a sterile formulation of the compounds of the invention can be dissolved and administered in a pharmaceutical excipient such as water-for-injection, 0.9% saline, or 5% glucose solution.

Conventional methods, known to those of ordinary skill in the art of medicine, can be used to administer the pharmaceutical formulations to a mammalian subject. The pharmaceutical formulations can be administered via oral, subcutaneous, intrapulmonary, transmucosal, intraperitoneal, intrauterine, sublingual, intrathecal, or intramuscular routes.

III. Nucleic Acid Molecules

As used herein a “nucleobase” refers to a heterocyclic base, such as, for example, a naturally occurring nucleobase (i.e., an A, T, G, C, or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for a naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases, and also derivative(s) and analog(s) thereof, including but not limited to, a purine or pyrimidine substituted by one or more of an alkyl, carboxyalkyl, amino, hydroxyl, halogen (i.e., fluoro, chloro, bromo, or iodo), thiol, or alkylthiol moiety. Preferred alkyl (e.g., alkyl, carboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcytosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenine, a 8-bromoadenine, a 8-hydroxyadenine, a 6-hydroxyaminopurine, a 6-thiopurine, a 4-(6-aminohexyl/cytosine), and the like. A nucleobase may be comprised in a nucleoside or nucleotide, using any chemical or natural synthesis method described herein or known to one of ordinary skill in the art. Such nucleobase may be labeled or it may be part of a molecule that is labeled and contains the nucleobase.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including, but not limited to, a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992, “DNA replication,” Freeman and Company, New York,).

As used herein, a “nucleotide” refers to a nucleoside further comprising a “backbone moiety.” A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, “Nucleotide Analogs: Synthesis and Biological Function,” Wiley, N.Y.).

Additional non-limiting examples of nucleosides, nucleotides, or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acid probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety, which may comprise a drug or label, to the 2′ carbon of an oligonucleotide to provide enhanced nuclease stability and the ability to deliver drugs or detection moieties; U.S. Pat. No. 5,223,618, which describes oligonucleotide analogs with a 2 or 3 carbon backbone linkage attaching the 4′ position and 3′ position of an adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probes; U.S. Pat. Nos. 5,378,825, 5,777,092, 5,623,070, 5,610,289 and 5,602,240, which describe oligonucleotides with a three or four atom linker moiety replacing phosphodiester backbone moiety used for improved nuclease resistance, cellular uptake and regulating RNA expression; U.S. Pat. No. 5,858,988, which describes a hydrophobic carrier agent attached to the 2′-O position of oligonucleotides to enhance their membrane permeability and stability; U.S. Pat. No. 5,214,136, which describes oligonucleotides conjugated to anthraquinone at the 5′ terminus that possesses enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; and U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; and U.S. Pat. Nos. 5,637,683, 6,251,666 (L-nucleotide substitutions), and 5,480,980 (7-deaza-2′ deoxyguanosine nucleotides and nucleic acid analogs thereof).

shRNA Mediated Suppression

Alternatively, certain of the nucleic acid molecules of the instant invention can be expressed within cells from eukaryotic promoters (e.g., Izant and Weintraub, 1985, Science, 229:345; McGarry and Lindquist, 1986, Proc. Natl. Acad. Sci., USA 83:399; Scanlon et al., 1991, Proc. Natl. Acad. Sci. USA, 88:10591-95; Kashani-Sabet et al., 1992, Antisense Res. Dev., 2:3-15; Dropulic et al., 1992, J. Virol., 66:1432-41; Weerasinghe et al., 1991, J. Virol., 65:5531-4; Ojwang et al., 1992, Proc. Natl. Acad. Sci. USA, 89:10802-06; Chen et al., 1992, Nucleic Acids Res., 20:4581 89; Sarver et al., 1990 Science, 247:1222-25; Thompson et al., 1995, Nucleic Acids Res., 23:2259; Good et al., 1997, Gene Therapy, 4:45). Those skilled in the art will realize that any nucleic acid can be expressed in eukaryotic cells from the appropriate DNA/RNA vector. The activity of such nucleic acids can be augmented by their release from the primary transcript by an enzymatic nucleic acid (Draper et al., International Application No WO 93/23569, and Sullivan et al., International Application No. WO 94/02595; Ohkawa et al., 1992, Nucleic Acids Symp. Ser., 27:15-6; Taira et al., 1991, Nucleic Acids Res., 19:5125-30; Ventura et al., 1993, Nucleic Acids Res., 21:3249-55; Chowrira et al., 1994, J. Biol. Chem. 269:25856). Gene therapy approaches specific to the CNS are described by Blesch et al., 2000, Drug News Perspect., 13:269-280; Peterson et al., 2000, Cent. Nerv. Syst. Dis., 485:508; Peel and Klein, 2000, J. Neurosci. Methods, 98:95-104; Hagihara et al., 2000, Gene Ther., 7:759-763; and Herrlinger et al., 2000, Methods Mol. Med. 35:287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see, for example, Couture et al., 1996, TIG. 12:510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells explanted from the patient or subject followed by reintroduction into the patient or subject, or by any other means that would allow for introduction into the desired target cell (for a review see Couture et al., 1996, TIG. 12:510).

In one aspect, the invention features an expression vector comprising a nucleic acid sequence encoding at least one of the nucleic acid molecules of the instant invention. The nucleic acid sequence encoding the nucleic acid molecule of the instant invention is operably linked in a manner which allows expression of that nucleic acid molecule.

In another aspect, the invention features an expression vector comprising:

a) a transcription initiation region (e.g., eukaryotic pol I, II, or III initiation region);
b) a transcription termination region (e.g., eukaryotic pol I, II, or III termination region);
c) a nucleic acid sequence encoding at least one of the nucleic acid molecules of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid molecule of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87:6743-7; Gao and Huang, 1993, Nucleic Acids Res., 21:2867-72; Lieber et al., 1993, Methods Enzymol., 217:47-66; Zhou et al., 1990, Mol. Cell. Biol., 10:4529-37).

Several investigators have demonstrated that nucleic acid molecules encoding shRNAs or microRNAs expressed from such promoters can function in mammalian cells (Brummelkamp et al., 2002, Science 296:550-553; Paddison et al., 2004, Nat. Methods 1:163-67; McIntyre and Fanning 2006 BMC Biotechnology (January 5) 6:1; Taxman et al., 2006 BMC Biotechnology (January 24) 6:7). The above shRNA or microRNA transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review, see Couture and Stinchcomb, 1996, supra).

In another aspect the invention features an expression vector comprising a nucleic acid sequence encoding at least one of the nucleic acid molecules of the invention, in a manner which allows expression of that nucleic acid molecule. The expression vector comprises in one embodiment: (a) a transcription initiation region; (b) a transcription termination region; (c) a nucleic acid sequence encoding at least one said nucleic acid molecule; and wherein said sequence is operably linked to said initiation region and said termination region, in a manner which allows expression and/or delivery of said nucleic acid molecule.

In another embodiment, the expression vector comprises: (a) a transcription initiation region; (b) a transcription termination region; (c) an open reading frame; (d) a nucleic acid sequence encoding at least one said nucleic acid molecule, wherein said sequence is operably linked to the 3′-end of said open reading frame; and wherein said sequence is operably linked to said initiation region, said open reading frame, and said termination region, in a manner which allows expression and/or delivery of said nucleic acid molecule. In yet another embodiment, the expression vector comprises: (a) a transcription initiation region; (b) a transcription termination region; (c) an intron; (d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame, and said termination region, in a manner which allows expression and/or delivery of said nucleic acid molecule.

IV. Modifield siNA Molecules

Any of the siNA constructs described herein can be evaluated and modified as described below.

An siNA construct may be susceptible to cleavage by an endonuclease or exonuclease, such as, for example, when the siNA construct is introduced into the body of a subject. Methods can be used to determine sites of cleavage, e.g., endo- and exonucleolytic cleavage on an RNAi construct and to determine the mechanism of cleavage. An siNA construct can be modified to inhibit such cleavage.

Exemplary modifications include modifications that inhibit endonucleolytic degradation, including the modifications described herein. Particularly favored modifications include: 2′ modification, e.g., a 2′-O-methylated nucleotide or 2′-deoxy nucleotide (e.g., 2′ deoxy-cytodine), or a 2′-fluoro, difluorotoluoyl, 5-Me-2′-pyrimidines, 5-allyamino-pyrimidines, 2′-O-methoxyethyl, 2′-hydroxy, or 2′-ara-fluoro nucleotide, or a locked nucleic acid (LNA), extended nucleic acid (ENA), hexose nucleic acid (HNA), or cyclohexene nucleic acid (CeNA). In one embodiment, the 2′ modification is on the uridine of at least one 5′-uridine-adenine-3′ (5′-UA-3′) dinucleotide, at least one 5′-uridine-guanine-3′ (5′-UG-3′) dinucleotide, at least one 5′-uridine-uridine-3′ (5′-UU-3′) dinucleotide, or at least one 5′-uridine-cytidine-3′ (5′-UC-3′) dinucleotide, or on the cytidine of at least one 5′-cytidine-adenine-3′ (5′-CA-3′) dinucleotide, at least one 5′-cytidine-cytidine-3′ (5′-CC-3′) dinucleotide, or at least one 5′-cytidine-uridine-3′ (5′-CU-3′) dinucleotide. The 2′ modification can also be applied to all the pyrimidines in an siNA construct. In one preferred embodiment, the 2′ modification is a 2′O-Me modification on the sense strand of an siNA construct. In a more preferred embodiment, the 2′ modification is a 2′ fluoro modification, and the 2′ fluoro is on the sense (passenger) or antisense (guide) strand or on both strands.

Modification of the backbone, e.g., with the replacement of an 0 with an S, in the phosphate backbone, e.g., the provision of a phosphorothioate modification can be used to inhibit endonuclease activity. In some embodiments, an siNA construct has been modified by replacing one or more ribonucleotides with deoxyribonucleotides. Preferably, adjacent deoxyribonucleotides are joined by phosphorothioate linkages, and the siNA construct does not include more than four consecutive deoxyribonucleotides on the sense or the antisense strands. Replacement of the U with a C5 amino linker; replacement of an A with a G (sequence changes are preferred to be located on the sense strand and not the antisense strand); or modification of the sugar at the 2′, 6′, 7′, or 8′ position can also inhibit endonuclease cleavage of the siNA construct. Preferred embodiments are those in which one or more of these modifications are present on the sense but not the antisense strand, or embodiments where the antisense strand has fewer of such modifications.

Exemplary modifications also include those that inhibit degradation by exonucleases. In one embodiment, an siNA construct includes a phosphorothioate linkage or P-alkyl modification in the linkages between one or more of the terminal nucleotides of an siNA construct. In another embodiment, one or more terminal nucleotides of an siNA construct include a sugar modification, e.g., a 2′ or 3′ sugar modification. Exemplary sugar modifications include, for example, a 2′-O-methylated nucleotide, 2′-deoxy nucleotide (e.g., deoxy-cytodine), 2′-deoxy-2′-fluoro (2′-F) nucleotide, 2′-O-methoxyethyl (2′-O-MOE), 2′-O-aminopropyl (2′-O-AP), 2′-0-N-methylacetamido (2′-O—NMA), 2′-O-dimethylaminoethlyoxyethyl (2′-DMAEOE), 2′-O-dimethylaminoethyl (2′-O-DMAOE), 2′-O-dimethylaminopropyl (2′-O-AP), 2′-hydroxy nucleotide, or a 2′-ara-fluoro nucleotide, or a locked nucleic acid (LNA), extended nucleic acid (ENA), hexose nucleic acid (HNA), or cyclohexene nucleic acid (CeNA). A 2′ modification is preferably 2′OMe, more preferably, 2′ fluoro.

The modifications described to inhibit exonucleolytic cleavage can be combined onto a single siNA construct. For example, in one embodiment, at least one terminal nucleotide of an siNA construct has a phosphorothioate linkage and a 2′ sugar modification, e.g., a 2′F or 2′OMe modification. In another embodiment, at least one terminal nucleotide of an siNA construct has a 5′ Me-pyrimidine and a 2′ sugar modification, e.g., a 2′F or 2′OMe modification.

To inhibit exonuclease cleavage, an siNA construct can include a nucleobase modification, such as a cationic modification, such as a 3′-abasic cationic modification. The cationic modification can be, e.g., an alkylamino-dT (e.g., a C6 amino-dT), an allylamino conjugate, a pyrrolidine conjugate, a pthalamido or a hydroxyprolinol conjugate, on one or more of the terminal nucleotides of the siNA construct. In one embodiment, an alkylamino-dT conjugate is attached to the 3′ end of the sense or antisense strand of an RNAi construct. In another embodiment, a pyrrolidine linker is attached to the 3′ or 5′ end of the sense strand, or the 3′ end of the antisense strand. In one embodiment, an allyl amine uridine is on the 3′ or 5′ end of the sense strand, and not on the 5′ end of the antisense strand.

In one embodiment, the siNA construct includes a conjugate on one or more of the terminal nucleotides of the siNA construct. The conjugate can be, for example, a lipophile, a terpene, a protein binding agent, a vitamin, a carbohydrate, a retinoid, or a peptide. For example, the conjugate can be naproxen, nitroindole (or another conjugate that contributes to stacking interactions), folate, ibuprofen, cholesterol, retinoids, PEG, or a C5 pyrimidine linker. In other embodiments, the conjugates are glyceride lipid conjugates (e.g., a dialkyl glyceride derivative), vitamin E conjugates, or thio-cholesterols. In one embodiment, conjugates are on the 3′ end of the antisense strand, or on the 5′ or 3′ end of the sense strand and the conjugates are not on the 3′ end of the antisense strand and on the 3′ end of the sense strand.

In one embodiment, the conjugate is naproxen, and the conjugate is on the 5′ or 3′ end of the sense or antisense strands. In one embodiment, the conjugate is cholesterol, and the conjugate is on the 5′ or 3′ end of the sense strand and not present on the antisense strand. In some embodiments, the cholesterol is conjugated to the siNA construct by a pyrrolidine linker, or serinol linker, aminooxy, or hydroxyprolinol linker. In other embodiments, the conjugate is a dU-cholesterol, or cholesterol is conjugated to the siNA construct by a disulfide linkage. In another embodiment, the conjugate is cholanic acid, and the cholanic acid is attached to the 5′ or 3′ end of the sense strand, or the 3′ end of the antisense strand. In one embodiment, the cholanic acid is attached to the 3′ end of the sense strand and the 3′ end of the antisense strand. In another embodiment, the conjugate is PEG5, PEG20, naproxen or retinol.

In another embodiment, one or more terminal nucleotides have a 2′-5′ linkage. In certain embodiments, a 2′-5′ linkage occurs on the sense strand, e.g., the 5′ end of the sense strand.

In one embodiment, an siNA construct includes an L-sugar, preferably at the 5′ or 3′ end of the sense strand.

In one embodiment, an siNA construct includes a methylphosphonate at one or more terminal nucleotides to enhance exonuclease resistance, e.g., at the 3′ end of the sense or antisense strands of the construct.

In one embodiment, an siRNA construct has been modified by replacing one or more ribonucleotides with deoxyribonucleotides. In another embodiment, adjacent deoxyribonucleotides are joined by phosphorothioate linkages. In one embodiment, the siNA construct does not include more than four consecutive deoxyribonucleotides on the sense or the antisense strands. In another embodiment, all of the ribonucleotides have been replaced with modified nucleotides that are not ribonucleotides.

In some embodiments, an siNA construct having increased stability in cells and biological samples includes a difluorotoluoyl (DFT) modification, e.g., 2,4-difluorotoluoyl uracil, or a guanidine to inosine substitution.

The methods can be used to evaluate a candidate siNA, e.g., a candidate siRNA construct, which is unmodified or which includes a modification, e.g., a modification that inhibits degradation, targets the dsRNA molecule, or modulates hybridization. Such modifications are described herein. A cleavage assay can be combined with an assay to determine the ability of a modified or non-modified candidate to silence the target transcript. For example, one might (optionally) test a candidate to evaluate its ability to silence a target (or off-target sequence), evaluate its susceptibility to cleavage, modify it (e.g., as described herein, e.g., to inhibit degradation) to produce a modified candidate, and test the modified candidate for one or both of the ability to silence and the ability to resist degradation. The procedure can be repeated. Modifications can be introduced one at a time or in groups. It will often be convenient to use a cell-based method to monitor the ability to silence a target RNA. This can be followed by a different method, e.g., a whole animal method, to confirm activity.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990, Nature 344:565; Pieken et al., 1991, Science 253:314; Usman and Cedergren, 1992, Trends in Biochem. Sci. 17:334; Burgin et al., 1996, Biochemistry, 35:14090; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Vargeese et al., U.S. Publication No. 2006/021733). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

Chemically modified siNA molecules for use in modulating or attenuating expression of two or more genes down-regulated by one or more miR-192 family member(s) are also within the scope of the invention. Described herein are isolated siNA agents, e.g., RNA molecules (chemically modified or not, double-stranded, or single-stranded) that mediate RNAi to inhibit expression of two or more genes that are down-regulated by one or more miR-192 family member.

The siNA agents discussed herein include otherwise unmodified RNA as well as RNAs which have been chemically modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., 1994, Nucleic Acids Res. 22:2183-2196. Such rare or unusual RNAs, though often termed modified RNAs (apparently because they are typically the result of a post-transcriptional modification) are within the term unmodified RNA, as used herein.

Modified RNA, as used herein, refers to a molecule in which one or more of the components of the nucleic acid, namely sugars, bases, and phosphate moieties that are the components of the RNAi duplex, are different from that which occur in nature, preferably different from that which occurs in the human body. While they are referred to as “modified RNAs,” they will of course, because of the modification, include molecules which are not RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Examples of all of the above are discussed herein.

Modifications described herein can be incorporated into any double-stranded RNA and RNA-like molecule described herein, e.g., an siNA construct. It may be desirable to modify one or both of the antisense and sense strands of an siNA construct. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid, but in many, and in fact in most, cases it will not.

By way of example, a modification may occur at a 3′ or 5′ terminal position, may occur in a terminal region, e.g., at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, a modification may occur on an internal residue to the exclusion of adjacent residues. In some cases, the sense and antisense strands will have the same modifications, or the same class of modifications, but in other cases the sense and antisense strands will have different modifications, e.g., in some cases it may be desirable to modify only one strand, e.g., the sense strand. In some cases, the sense strand may be modified, e.g., capped in order to promote insertion of the anti-sense strand into the RISC complex.

Other suitable modifications that can be made to a sugar, base, or backbone of an siNA construct are described in U.S. Publication Nos. 2006/0217331 and 2005/0020521, International Publication Nos. WO2003/70918 and WO2005/019453, and International Application No. PCT/US2004/01193. An siNA construct can include a non-naturally occurring base, such as the bases described in any one of the above mentioned references. See also International Application No. PCT/US2004/011822. An siNA construct can also include a non-naturally occurring sugar, such as a non-carbohydrate cyclic carrier molecule. Exemplary features of non-naturally occurring sugars for use in siNA agents are described in International Application No. PCT/US2004/11829.

Two prime objectives for the introduction of modifications into siNA constructs of the invention is their stabilization towards degradation in biological environments and the improvement of pharmacological properties, e.g., pharmacodynamic properties. There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, and nucleotide base modifications (for a review see Usman and Cedergren, 1992, TIBS 17:34; Usman et al., 1994, Nucleic Acids Symp. Ser. 31:163; Burgin et al., 1996, Biochemistry 35:14090). Sugar modification of nucleic acid molecules has been extensively described in the art (see Eckstein et al., International Publication No. WO 92/07065; Perrault et al., 1990, Nature, 344:565-568; Pieken et al., 1991, Science 253:314-317; Usman and Cedergren, 1992, Trends in Biochem. Sci. 17:334-339; Usman et al., International Publication No. WO 93/15187; Sproat, U.S. Pat. No. 5,334,711; Beigelman et al., 1995, J. Biol. Chem., 270:25702; Beigelman et al., International Publication No. WO 97/26270; Beigelman et al., U.S. Pat. No. 5,716,824; Usman et al., U.S. Pat. No. 5,627,053; Woolf et al., International Publication No. WO 98/13526; Thompson et al., U.S. Ser. No. 60/082,404, which was filed on Apr. 20, 1998; Karpeisky et al., 1998, Tetrahedron Lett. 39:1131; Earnshaw and Gait, 1998, Biopolymers (Nucleic Acid Sciences) 48:39-55; Verma and Eckstein, 1998, Annu. Rev. Biochem., 67:99-134; and Burlina et al., 1997, Bioorg. Med. Chem., 5:1999-2010). Such publications describe general methods and strategies to determine the location of incorporation of sugar, base, and/or phosphate modifications and the like, into nucleic acid molecules without modulating catalysis. In view of such teachings, similar modifications can be used as described herein to modify the siNA molecules of the instant invention so long as the ability of siNA to promote RNAi in cells is not significantly inhibited.

Modifications may be modifications of the sugar-phosphate backbone. Modifications may also be modifications of the nucleoside portion. Optionally, the sense strand is an RNA or RNA strand comprising 5%, 10%, 20%, 30%, 40%, 50%, 60%, 70%, 80%, 90%, or 100% modified nucleotides. In one embodiment, the sense polynucleotide is an RNA strand comprising a plurality of modified ribonucleotides. Likewise, in other embodiments, the RNA antisense strand comprises one or more modifications. For example, the RNA antisense strand may comprise no more than 5%, 10%, 20%, 30%, 40%, 50%, or 75% modified nucleotides. The one or more modifications may be selected so as to increase the hydrophobicity of the double-stranded nucleic acid, in physiological conditions, relative to an unmodified double-stranded nucleic acid having the same designated sequence.

In certain embodiments, the siNA construct comprising the one or more modifications has a log P value at least 0.5 log P units less than the log P value of an otherwise identical unmodified siRNA construct. In another embodiment, the siNA construct comprising the one or more modifications has at least 1, 2, 3, or even 4 log P units less than the log P value of an otherwise identical unmodified siRNA construct. The one or more modifications may be selected so as to increase the positive charge (or increase the negative charge) of the double-stranded nucleic acid, in physiological conditions, relative to an unmodified double-stranded nucleic acid having the same designated sequence. In certain embodiments, the siNA construct comprising the one or more modifications has an isoelectric pH (pI) that is at least 0.25 units higher than the otherwise identical unmodified siRNA construct. In another embodiment, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety, and a 2′-deoxy-2′-fluoride moiety.

In certain embodiments, the RNAi construct is a hairpin nucleic acid that is processed to an siRNA inside a cell. Optionally, each strand of the double-stranded nucleic acid may be 19-100 base pairs long, and preferably 19-50 or 19-30 base pairs long.

An siNAi construct can include an internucleotide linkage (e.g., the chiral phosphorothioate linkage) useful for increasing nuclease resistance. In addition, or in the alternative, an siNA construct can include a ribose mimic for increased nuclease resistance. Exemplary internucleotide linkages and ribose mimics for increased nuclease resistance are described in International Application No. PCT/US2004/07070.

An siRNAi construct can also include ligand-conjugated monomer subunits and monomers for oligonucleotide synthesis. Exemplary monomers are described, for example, in U.S. patent application Ser. No. 10/916,185.

An siNA construct can have a ZXY structure, such as is described in co-owned International Application No. PCT/US2004/07070. Likewise, an siNA construct can be complexed with an amphipathic moiety. Exemplary amphipathic moieties for use with siNA agents are described in International Application No. PCT/US2004/07070.

The sense and antisense sequences of an siNA construct can be palindromic. Exemplary features of palindromic siNA agents are described in PCT Application No. PCT/US2004/07070.

In another embodiment, the siNA construct of the invention can be complexed to a delivery agent that features a modular complex. The complex can include a carrier agent linked to one or more of (preferably two or more, more preferably all three of): (a) a condensing agent (e.g., an agent capable of attracting, e.g., binding, a nucleic acid, e.g., through ionic or electrostatic interactions); (b) a fusogenic agent (e.g., an agent capable of fusing and/or being transported through a cell membrane); and (c) a targeting group, e.g., a cell or tissue targeting agent, e.g., a lectin, glycoprotein, lipid, or protein, e.g., an antibody, that binds to a specified cell type. iRNA agents complexed to a delivery agent are described in International Application No. PCT/US2004/07070.

The siNA construct of the invention can have non-canonical pairings, such as between the sense and antisense sequences of the iRNA duplex. Exemplary features of non-canonical iRNA agents are described in International Application No. PCT/US2004/07070.

In one embodiment, nucleic acid molecules of the invention include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see for example, Lin and Matteucci, 1998, J. Am. Chem. Soc. 120:8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more (e.g., about 1, 2, 3, 4, 5, 6, 7, 8, 9, 10, or more) LNA “locked nucleic acid” nucleotides such as a 2′,4′-C methylene bicyclo nucleotide (see, for example, Wengel et al., International Publication Nos. WO 00/66604 and WO 99/14226).

An siNA agent of the invention can be modified to exhibit enhanced resistance to nucleases. An exemplary method proposes identifying cleavage sites and modifying such sites to inhibit cleavage. An exemplary dinucleotide 5′-UA-3′,5′-UG-3′,5′-CA-3′, 5′-UU-3′, or 5′-CC-3′ as disclosed in International Application No. PCT/US2005/018931 may serve as a cleavage site.

For increased nuclease resistance and/or binding affinity to the target, a siRNA agent, e.g., the sense and/or antisense strands of the iRNA agent, can include, for example, 2′-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl, or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

“Deoxy” modifications include hydrogen (i.e., deoxyribose sugars, which are of particular relevance to the overhang portions of partially ds RNA); halo (e.g., fluoro); amino (e.g., NH₂, alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with, e.g., an amino functionality. In one embodiment, the substituents are 2′-methoxyethyl, 2′-OCH₃, 2′-O-allyl, 2′-C-allyl, and 2′-fluoro.

In another embodiment, to maximize nuclease resistance, the 2′ modifications may be used in combination with one or more phosphate linker modifications (e.g., phosphorothioate). The so-called “chimeric” oligonucleotides are those that contain two or more different modifications.

In certain embodiments, all the pyrimidines of a siNA agent carry a 2′-modification, and the molecule therefore has enhanced resistance to endonucleases. Enhanced nuclease resistance can also be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. The siNA agent can include at least 2, at least 3, at least 4, or at least 5 of such dinucleotides. In some embodiments, the 5′-most pyrimidines in all occurrences of the sequence motifs 5′-UA-3′, 5′-CA-3′,5′-UU-3′, and 5′-UG-3′ are 2′-modified nucleotides. In other embodiments, all pyrimidines in the sense strand are 2′-modified nucleotides, and the 5′-most pyrimidines in all occurrences of the sequence motifs include 5′-UA-3′ and 5′-CA-3′. In one embodiment, all pyrimidines in the sense strand are 2′-modified nucleotides, and the 5′-most pyrimidines in all occurrences of the sequence motifs 5′-UA-3′, 5′-CA-3′, 5′-UU-3′, and 5′-UG-3′ are 2′-modified nucleotides in the antisense strand. The latter patterns of modifications have been shown to maximize the contribution of the nucleotide modifications to the stabilization of the overall molecule towards nuclease degradation, while minimizing the overall number of modifications required to achieve a desired stability, see International Application No. PCT/US2005/018931. Additional modifications to enhance resistance to nucleases may be found in U.S. Publication No. 2005/0020521, and International Application Publication Nos. WO2003/70918 and WO2005/019453.

The inclusion of furanose sugars in the oligonucleotide backbone can also decrease endonucleolytic cleavage. Thus, in one embodiment, the siNA of the invention can be modified by including a 3′ cationic group, or by inverting the nucleoside at the 3′-terminus with a 3′-3′ linkage. In another alternative, the 3′-terminus can be blocked with an aminoalkyl group, e.g., a 3′ C5-aminoalkyl dT. Other 3′ conjugates can inhibit 3′-5′ exonucleolytic cleavage. While not being bound by theory, a 3′ conjugate, such as naproxen or ibuprofen, may inhibit exonucleolytic cleavage by sterically blocking the exonuclease from binding to the 3′-end of oligonucleotide. Even small alkyl chains, aryl groups, heterocyclic conjugates, or modified sugars (D-ribose, deoxyribose, glucose, etc.) can block 3′-5′-exonucleases.

Similarly, 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′-end of oligonucleotide. Even small alkyl chains, aryl groups, heterocyclic conjugates, or modified sugars (D-ribose, deoxyribose, glucose, etc.) can block 3′-5′-exonucleases.

An alternative approach to increasing resistance to a nuclease by an siNA molecule proposes including an overhang to at least one or both strands of a duplex siNA. In some embodiments, the nucleotide overhang includes 1 to 4, preferably 2 to 3, unpaired nucleotides. In another embodiment, the unpaired nucleotide of the single-stranded overhang that is directly adjacent to the terminal nucleotide pair contains a purine base, and the terminal nucleotide pair is a G-C pair, or at least two of the last four complementary nucleotide pairs are G-C pairs. In other embodiments, the nucleotide overhang may have 1 or 2 unpaired nucleotides, and in an exemplary embodiment the nucleotide overhang may be 5′-GC-3′. In another embodiment, the nucleotide overhang is on the 3′-end of the antisense strand.

Thus, an siNA molecule can include monomers which have been modified so as to inhibit degradation, e.g., by nucleases, e.g., endonucleases or exonucleases, found in the body of a subject. These monomers are referred to herein as NRMs, or Nuclease Resistance promoting Monomers or modifications. In some cases these modifications will modulate other properties of the siNA agent as well, e.g., the ability to interact with a protein, e.g., a transport protein, e.g., serum albumin, or a member of the RISC, or the ability of the first and second sequences to form a duplex with one another or to form a duplex with another sequence, e.g., a target molecule.

While not wishing to be bound by theory, it is believed that modifications of the sugar, base, and/or phosphate backbone in an siNA agent can enhance endonuclease and exonuclease resistance, and can enhance interactions with transporter proteins and one or more of the functional components of the RISC complex. In some embodiments, the modification may increase exonuclease and endonuclease resistance and thus prolong the half-life of the siNA agent prior to interaction with the RISC complex, but at the same time does not render the siNA agent inactive with respect to its intended activity as a target mRNA cleavage directing agent. Again, while not wishing to be bound by any theory, it is believed that placement of the modifications at or near the 3′ and/or 5′-end of antisense strands can result in siNA agents that meet the preferred nuclease resistance criteria delineated above.

Modifications that can be useful for producing siNA agents that exhibit the nuclease resistance criteria delineated above may include one or more of the following chemical and/or stereochemical modifications of the sugar, base, and/or phosphate backbone, it being understood that the art discloses other methods as well that can achieve the same result:
(i) chiral (Sp) thioates. An NRM may include nucleotide dimers, enriched or pure, for a particular chiral form of a modified phosphate group containing a heteroatom at the nonbridging position, e.g., Sp or Rp, at the position X, where this is the position normally occupied by the oxygen. The atom at X can also be S, Se, Nr₂, or Br₃. When X is S, enriched or chirally pure Sp linkage is preferred. Enriched means at least 70, 80, 90, 95, or 99% of the preferred form.
(ii) attachment of one or more cationic groups to the sugar, base, and/or the phosphorus atom of a phosphate or modified phosphate backbone moiety. In some embodiments, these may include monomers at the terminal position derivatized at a cationic group. As the 5′-end of an antisense sequence should have a terminal —OH or phosphate group, this NRM is preferably not used at the 5′-end of an antisense sequence. The group should preferably be attached at a position on the base which minimizes interference with H bond formation and hybridization, e.g., away from the face which interacts with the complementary base on the other strand, e.g., at the 5′ position of a pyrimidine or a 7-position of a purine.
(iii) nonphosphate linkages at the termini. In some embodiments, the NRMs include non-phosphate linkages, e.g., a linkage of 4 atoms which confers greater resistance to cleavage than does a phosphate bond. Examples include 3′ CH2-NCH₃—O—CH₂-5′ and 3′ CH₂—NH—(O═)—CH₂-5′.
(iv) 3′-bridging thiophosphates and 5′-bridging thiophosphates. In certain embodiments, the NRMs can be included among these structures.
(v) L-RNA, 2′-5′ linkages, inverted linkages, and a-nucleosides. In certain embodiments, the NRMs include: L nucleosides and dimeric nucleotides derived from L-nucleosides; 2′-5′ phosphate, non-phosphate and modified phosphate linkages (e.g., thiophosphates, phosphoramidates, and boronophosphates); dimers having inverted linkages, e.g., 3′-3′ or 5′-5′ linkages; monomers having an alpha linkage at the 1′ site on the sugar, e.g., the structures described herein having an alpha linkage,
(vi) conjugate groups. In certain embodiments, the NRMs can include, e.g., a targeting moiety or a conjugated ligand described herein conjugated with the monomer, e.g., through the sugar, base, or backbone;
(vi) abasic linkages. In certain embodiments, the NRMs can include an abasic monomer, e.g., an abasic monomer as described herein (e.g., a nucleobaseless monomer); an aromatic or heterocyclic or polyheterocyclic aromatic monomer as described herein; and
(vii) 5′-phosphonates and 5′-phosphate prodrugs. In certain embodiments, the NRMs include monomers, preferably at the terminal position, e.g., the 5′ position, in which one or more atoms of the phosphate group is derivatized with a protecting group, which protecting group or groups are removed as a result of the action of a component in the subject's body, e.g., a carboxyesterase or an enzyme present in the subject's body. For example, a phosphate prodrug in which a carboxy esterase cleaves the protected molecule resulting in the production of a thioate anion which attacks a carbon adjacent to the O of a phosphate and resulting in the production of an unprotected phosphate.

“Ligand,” as used herein, means a molecule that specifically binds to a second molecule, typically a polypeptide or portion thereof, such as a carbohydrate moiety, through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity (e.g., agonists or antagonists), the term as used herein applies more generally.

One or more different NRM modifications can be introduced into a siNA agent or into a sequence of a siRNA agent. An NRM modification can be used more than once in a sequence or in a siRNA agent. As some NRMs interfere with hybridization, the total number incorporated should be such that acceptable levels of siNA agent duplex formation are maintained.

In some embodiments, NRM modifications are introduced into the terminal cleavage site or in the cleavage region of a sequence (a sense strand or sequence) which does not target a desired sequence or gene in the subject.

In most cases, the nuclease-resistance promoting modifications will be distributed differently depending on whether the sequence will target a sequence in the subject (often referred to as an antisense sequence) or will not target a sequence in the subject (often referred to as a sense sequence). If a sequence is to target a sequence in the subject, modifications which interfere with or inhibit endonuclease cleavage should not be inserted in the region which is subject to RISC mediated cleavage, e.g., the cleavage site or the cleavage region (as described in Elbashir et al., 2001, Genes and Dev. 15:188). Cleavage of the target occurs about in the middle of a 20 or 21 nt guide RNA, or about 10 or 11 nucleotides upstream of the first nucleotide which is complementary to the guide sequence. As used herein, “cleavage site” refers to the nucleotide on either side of the cleavage site, on the target, or on the iRNA agent strand which hybridizes to it. Cleavage region means a nucleotide within 1, 2, or 3 nucleotides of the cleavage site, in either direction.

Such modifications can be introduced into the terminal regions, e.g., at the terminal position, or within 2, 3, 4, or 5 positions of the terminus, of a sequence which targets, or a sequence which does not target, a sequence in the subject.

In general, an effective amount of the one or more compositions of the invention for treating a mammalian subject afflicted with cancer will be that amount necessary to inhibit mammalian cancer cell proliferation in situ. Those of ordinary skill in the art are well-schooled in the art of evaluating effective amounts of anti-cancer agents.

In some cases, the above-described treatment methods may be combined with known cancer treatment methods. The term “cancer treatment” as used herein, may include, but is not limited to, chemotherapy, radiotherapy, adjuvant therapy, surgery, or any combination of these and/or other methods. Particular forms of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

The molecules of the instant invention can be used as pharmaceutical agents. Pharmaceutical agents prevent, inhibit the occurrence of, or treat (alleviate a symptom to some extent, preferably all of the symptoms) a disease state in a subject.

The negatively charged polynucleotides of the invention (e.g., RNA, DNA or protein complex thereof) can be administered and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary, and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as lymphocytes and macrophages, is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13:16-26); biodegradable polymers, such as poly (DL-lactide-coglycolide) microspheres for sustained release delivery after implantation (Emerich, D. F., et al., 1999Cell Transplant, 8:47-58) Alkermes, Inc., Cambridge, Mass.; and loaded nanoparticles, such as those made of polybutylcyanoacrylate, which can deliver drugs across the blood brain barrier and can alter neuronal uptake mechanisms (Prog. Neuropsychopharmacol. Biol. Psychiatry, 23:941-949, 1999). Nanoparticles functionalized with lipids (lipid nanoparticles), such as lysine-containing nanoparticles with the surface functional groups modified with lipid chains may also be used for delivery of the nucleic acid molecules of the instant invention. Such lipid nanoparticles may be generated as described in Baigude, H., et al., ACS Chemical Biology 2(4):237-241 (2007), incorporated herein by reference. Other non-limiting examples of delivery strategies, including CNS delivery of the nucleic acid molecules of the instant invention, include material described in Boado et al., 1998, J. Pharm. Sci., 87:1308-1315; Tyler et al., 1999, FEBS Lett., 421:280-284; Pardridge et al., 1995, PNAS USA., 92:5592-5596; Boado, 1995, Adv. Drug Delivery Rev., 15:73-107; Aldrian-Herrada et al., 1998, Nucleic Acids Res., 26:4910-4916; and Tyler et al., 1999, PNAS USA., 96:7053-7058. All these references are hereby incorporated herein by reference in their entirety.

The invention also features the use of the composition comprising surface-modified liposomes containing poly(ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., 1995, Chem. Rev. 95:2601-2627; Ishiwata et al., 1995, Chem. Pharm. Bull. 43:1005-1011). Such liposomes have been shown to accumulate selectively in tumors, presumably by extravasation and capture in the neovascularized target tissues (Lasic et al., 1995, Science 267:1275-1276; Oku et al., 1995, Biochim. Biophys. Acta, 1238:86-90). The long-circulating liposomes enhance the pharmacokinetics and pharmacodynamics of DNA and RNA, particularly compared to conventional cationic liposomes which are known to accumulate in tissues of the MPS (Liu et al., 1995, J. Biol. Chem. 42:24864-24870; Choi et al., International Publication No. WO 96/10391; Ansell et al., International PCT Publication No. WO 96/10390; Holland et al., International Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro, ed., 1985), hereby incorporated by reference. For example, preservatives, stabilizers, dyes, and flavoring agents can be provided. These include sodium benzoate, sorbic acid, and esters of p-hydroxybenzoic acid. In addition, antioxidants and suspending agents can be used.

A pharmaceutically effective dose is the dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered, depending upon the potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques, and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and, if desired, other active ingredients. The pharmaceutical compositions containing nucleic acid molecules of the invention can be in a form suitable for oral use, for example, as tablets, troches, lozenges, aqueous or oily suspensions, dispersible powders or granules, emulsions, hard or soft capsules, or syrups or elixirs.

In some embodiments, the compositions are administered locally to a localized region of a subject, such as a tumor, via local injection.

Compositions intended for oral use can be prepared according to any method known in the art for the manufacture of pharmaceutical compositions, and such compositions can contain one or more sweetening agents, flavoring agents, coloring agents, or preservative agents in order to provide pharmaceutically elegant and palatable preparations. Tablets may contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch or alginic acid; binding agents, for example starch, gelatin, or acacia, and lubricating agents, for example magnesium stearate, stearic acid, or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate, or kaolin, or as soft gelatin capsules, wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients may include suspending agents, for example sodium carboxymethylcellulose, methylcellulose, hydropropyl-methylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate; or condensation products of ethylene oxide with long chain aliphatic alcohols, for example heptadecaethyleneoxycetanol; or condensation products of ethylene oxide with partial esters derived from fatty acids and a hexitol such as polyoxyethylene sorbitol monooleate; or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil, or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent, and one or more preservatives. Suitable dispersing or wetting agents or suspending agents are exemplified by those already mentioned above. Additional excipients, for example sweetening, flavoring, and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example, gum acacia or gum tragacanth; naturally-occurring phosphatides, for example, soy bean, lecithin, and esters or partial esters derived from fatty acids and hexitol; anhydrides, for example, sorbitan monooleate; and condensation products of the said partial esters with ethylene oxide, for example, polyoxyethylene sorbitan monooleate. The emulsions can also contain sweetening and flavoring agents.

Syrups and elixirs can be formulated with sweetening agents, for example, glycerol, propylene glycol, sorbitol, glucose, or sucrose. Such formulations can also contain a demulcent, a preservative, and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels on the order of from about 0.1 mg to about 140 mg per kilogram of body weight per day are useful in the treatment of the above-indicated conditions (about 0.5 mg to about 7 g per patient or subject per day). The amount of active ingredient that can be combined with the carrier materials to produce a single dosage form varies depending upon the host to be treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, rate of excretion, drug combination, and the severity of the particular disease undergoing therapy.

For administration to non-human animals, the composition can also be added to the animal's feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat a disease or condition can increase the beneficial effects while reducing the presence of side effects.

The following examples merely illustrate the best mode now contemplated for practicing the invention, but should not be construed to limit the invention.

### Example 1

This Example demonstrates that miR-192 and miR-215 are upregulated in response to genotoxic stress.

Rationale:

It has been reported that miR-34a is strongly induced by p53 activation from genotoxic stress (He, L., et al., Nature 447:1130-1134 (2007)). In order to characterize other microRNAs that may be similarly regulated, microRNA expression was measured in a p53 matched pair cell line (p53 wild type or p53−/−) following treatment with the DNA damaging agent doxorubicin.

Methods:

TOV21G cells and A549 cells were obtained from the American Type Culture Collection (ATCC). TOV21G p53 and A549 p53 matched pair cell lines were created by stably infecting TOV21G and A549 cells with a lentivirus encoding either H1-term or p53 shRNA. Cells were cultured in Dulbecco's Modified Eagles Medium, supplemented with 10% fetal bovine serum, streptomycin, penicillin and L-glutamine.

MicroRNA expression was measured in the p53 matched cell lines (p53+/+ or p53−/− sh) either untreated or following treatment with various doses of the DNA damaging agent adriamycin (0, 10, 50 and 200 nM adriamycin). Forty-eight hours post treatment, RNA was harvested and the expression levels of miR-34a, miR-192, miR-215 and p21 were determined by quantitative RT-PCR with Taqman analysis carried out as described in Raymond C. K. et al., RNA 11: 1737-1744 (2005).

Results:

FIG. 2A graphically illustrates the fold change (as compared to the untreated cells) of miR-192, miR-215 and miR-34a expression levels in either wild type A549 cells (p53+/+), or A549 (p53−/−) cells following treatment with 0, 10, 50 or 200 nM adriamycin. FIG. 2B graphically illustrates the fold change (as compared to the untreated cells) in miR-192, miR-215 and miR-34a expression levels in either wild type TOV21G cells (p53+/+) or TOV21G (p53−/−) cells following treatment with 0, 10, 50 or 200 nM adriamycin.

As shown in FIG. 2A and FIG. 2B, up-regulation of miR-192, miR-215 and miR-34a was observed in both A549 cells and TOV21G cells after exposure to the DNA damaging agent adriamycin. As further shown in FIG. 2A and FIG. 2B, similar to miR-34a, the expression of miR-192 and miR-215 is upregulated in a dose dependent manner in wild type (p53+/+) cells but not in p53 deficient cells. FIG. 2C graphically illustrates the fold change (as compared to wild type untreated cells) of p21 expression levels (control) in matched pairs of A549 cells and TOV21G cells wild type (p53+/+) or p53 kd−/− following treatment with 0, 10, 50 or 200 nM adriamycin. The knockdown efficiency of p53 was functionally demonstrated by lack of p21 induction in response to adriamycin, as shown in FIG. 2C.

From these results, it was concluded that miR-192 and miR-215 are induced by p53 activity. The function of miR-192/miR-215 was further investigated through gene expression profiling and cell cycle analysis, as described in Example 2.

### Example 2

This Example demonstrates that transcripts regulated by miR-192/miR-215 are highly enriched for regulators of cell cycle progression.

Rationale: MicroRNAs down-regulate gene expression by inhibiting translation of their target transcripts and/or mediating the degradation of these transcripts. To better understand the function of the miR-192/miR-215 family, gene expression profiling experiments were performed.

Methods:

Synthetic duplex mimetics of miR-192 and miR-215 (sequences shown in TABLE 1) or a control target Luciferase siRNA (Luc), shown in TABLE 1, were transfected (10 nM final concentration) into HCT116 DICER^{ex5}, a human colorectal cancer cell line with hypomorphic DICER function (Cummins, J. M., et al., PNAS 103:3687-3692 (2006)). The transfections were carried out using Lipofectamine RNAiMax (Invitrogen) per the manufacturer's instructions. At 10 and 24 hours post-transfection, total RNA was extracted and gene expression profiling was carried out with the Agilent 44K microarray in comparison to RNA isolated from mock-transfected HCT116DICER^{ex5} cells. Microarray analysis was carried out as described in Linsley, P. S., et al., Mol Cell Biol 27:2240-2252 (2007). Gene expression data analysis was done either with the Rosetta Resolver gene expression analysis software (Version 7.1 Rosetta Biosoftware). The downregulated gene set was annotated by the Gene Ontology database.

TABLE 1
Synthetic miR-192 and miR-215 Oligonucleotide Sequences

| siRNA, | | | | |
|---|---|---|---|---|
| miRNA or | | SEQ | | SEQ |
| mismatch | Guide strand/mature | ID | Passenger strand | ID |
| miRNA | (5′ to 3′) | NO: | (5′ to 3′) | NO: |
| miR-192 (wt) | CUGACCUAUGAAUUGACAGCC | 1 | CUGUCAAUUCAUAGGUCUUAU | 7 |
| miR-192(mm4.5 | CUGCACUAUGAAUUGACAGCC | 8 | CUGUCAAUUCAUAGUGCUGAU | 9 |
| mutant) | | | | |
| miR-215 (wt) | AUGACCUAUGAAUUGACAGAC | 4 | CUGUCAAUUCAUAGGUCUUAU | 10 |
| luciferase | UCGAAGUAUUCCGCGUACGdT | 11 | CGUACGCGGAAUACUUCGAdTdT | 12 |
| siRNA | dT | | | |

Results:

In order to study the proximal primary effect of miR-192 and miR-215 expression on gene expression in the transfected cells, the analysis was focused on down-regulated transcripts. A heatmap was generated of the microarray data in order to identify transcripts that were down-regulated in response to miR-192 and/or miR-215, where the level of expression was shown in color. The color bar represented log 10 expression ratios (samples from transfected cells/samples from mock-transfected cells) of −1.0 (teal) to +1.0 (magenta). Microarray analysis identified a set of sequences (>1000) were identified as direct miR-192/miR-215 targets as well as indirect secondary effectors that were downregulated by miR-192/miR-215, with miR-192 and miR-215 transfected cells demonstrating virtually indistinguishable expression profiles (data not shown). As shown in FIG. 1, miR-192 (SEQ ID NO:1) has a seed region sequence (SEQ ID NO:3, underlined), that is nearly identical to the seed region sequence (SEQ ID NO:6, underlined), of miR-215 (SEQ ID NO:4).

The gene set identified as downregulated in miR-192/miR-215 transfected cells was then queried for enrichment in known members of established biological pathways. The set of sequences (>1000) that were identified as direct miR-192/miR-215 targets, as well as indirect secondary effectors that were downregulated by miR-192/miR-215, were annotated in the gene ontology biological processes database with the term “mitotic cell cycle” or “cell cycle.”

To focus on the direct targets of miR-192/miR-215, the >1000 sequence set was then queried for genes that contained the miR-192/miR-215 seed hexamer complement 5′AGGTCA3′ (SEQ ID NO:379) in their 3′ untranslated regions (3′ UTRs). It was determined that the 3′ UTRs of the observed miR-192/miR-215 downregulated transcripts were highly enriched with hexamer sequences complementary to the seed region of miR-192/miR-215, with an E value for hexamer enrichment, (likelihood that the hexamer enrichment was due to chance), was determined to be <1e⁻⁸³, as shown in TABLE 2. As shown in TABLE 2, annotation of this set of 62 genes report the top ranked category as “cell cycle” with a significant expectation (E value<1e⁻³¹).

TABLE 2
Statistical analysis of downregulated transcripts in
miR-192/miR-215 transfected cells

| Feature of Interest | Statistical Analysis |
|---|---|
| Hexamer enrichment in the 3′ UTR | E < 1e⁻⁸³ |
| Annotation of “mitotic cell cycle: | E < 1e⁻¹¹ |
| Annotation of 62 Genes downregulated by | E < 1e⁻³¹ |
| 10 hours post transfection and contain | |
| seed region as “cell cycle” | |

Through this analysis, a set of 62 genes that were down-regulated by miR-192/miR-215 expression as early as 10 hours as well as at 24 hours post-transfection, that contained at least one miR-192/miR-215 seed hexamer complement 5′AGGTCA3′ (SEQ ID NO:379) in their 3′ UTR were identified, as shown below in TABLE 3.

TABLE 3
Set of 62 Genes downregulated in HCT116 DICER^{ex5} Cells
by miR-192/miR-215 that contain 3′ UTR matches for miR-192
hexamer (SEQ ID NO: 379)

| | GenBank | miR-192 | miR-192 | miR-215 | miR-215 |
|---|---|---|---|---|---|
| | Reference | 10 hr | 24 hr | 10 hr | 24 hr |
| Gene Symbol | Number¹ | (fold change) | (fold change) | (fold change) | (fold change) |
| BCL2 | NM_000633 | −1.1 | −1.46 | −1.09 | −1.59 |
| BRCA1 | NM_007300 | −1.02 | −1.35 | −1.04 | −1.43 |
| CDC14A | NM_003672 | −1.02 | −1.16 | −1.05 | −1.2 |
| CDC7 | hCT2310901 | −1.63 | −2.05 | −1.54 | −2.04 |
| CDC73 | NM_024529 | −1.2 | −1.17 | −1.04 | −1.1 |
| CRK | NM_005206 | −1.42 | −1.44 | −1.43 | −1.52 |
| CTCF | NM_006565 | −1.59 | −1.78 | −1.53 | −1.66 |
| CUL5 | NM_003478 | −1.16 | −1.35 | −1.25 | −1.37 |
| DBF4B | NM_025104 | −1.12 | −1.33 | −1.07 | −1.33 |
| DEK | NM_003472 | −1.21 | −1.47 | −1.19 | −1.42 |
| DLG5 | NM_004747 | −1.99 | −2.22 | −1.89 | −2.03 |
| DTL | NM_016448 | −1.11 | −1.38 | −1.19 | −1.46 |
| EMP1 | NM_001423 | −1.28 | −1.66 | 1.11 | 1.04 |
| ERCC3 | NM_000122 | −1.3 | −1.6 | −1.25 | −1.36 |
| EXTL2 | NM_001439 | −1.2 | −1.5 | −1.32 | −1.59 |
| FGF2 | NM_002006 | −1.48 | −1.83 | −1.67 | −2.12 |
| HOXA10 | NM_153715 | −1.7 | −2.3 | −1.74 | −2.57 |
| HRH1 | NM_001098213 | −1.36 | −1.77 | −1.52 | −1.63 |
| KIF14 | NM_014875 | −1.03 | −1.42 | 1.01 | −1.53 |
| KIF23 | NM_004856 | −1.01 | −1.34 | 1.01 | −1.36 |
| LMNB2 | NM_032737 | −1.38 | −1.82 | −1.55 | −1.92 |
| MACF1 | NM_012090 | −1.3 | −1.64 | −1.17 | −1.55 |
| MAD2L | NM_002358 | −1.18 | −1.53 | −1.31 | −1.84 |
| MCM10 | NM_018518 | −1.77 | −2.37 | −1.97 | −2.67 |
| MCM3 | NM_002388 | −1.51 | −1.91 | −1.56 | −1.96 |
| MCM6 | NM_005915 | −1.01 | −1.33 | −1.04 | −1.4 |
| MIS12 | NM_024039 | −1.25 | −1.73 | −1.28 | −1.77 |
| MKI67 | NM_002417 | −1.08 | −1.4 | −1.04 | −1.52 |
| MPHOSPH1 | NM_016195 | −1.32 | −1.87 | −1.46 | −1.94 |
| MTSS1 | NM_014751 | −1.06 | −1.47 | −1.07 | −1.29 |
| NBN | NM_002485 | −1.49 | −1.27 | −1.39 | −1.41 |
| NCAPH | NM_015341 | 1.01 | −1.32 | −1.02 | −1.32 |
| NEK1 | AL050385 | −1.12 | −1.47 | −1.16 | −1.35 |
| NFIB | AL110126 | −1.47 | 1.82 | −1.38 | −1.91 |
| NFYA | HSS00227787 | −1.22 | −1.35 | −1.25 | −1.18 |
| NIN | Contig55214_RC | −1.1 | 1.38 | −1.12 | −1.42 |
| PAFAH1B1 | NM_000430 | −1.83 | −2.22 | −1.31 | −1.6 |
| PAPD5 | Contig41078_RC | −1.32 | −1.44 | −1.31 | −1.22 |
| PFTK1 | NM_012395 | 1.03 | −1.33 | 1.05 | −1.28 |
| PIM1 | NM_002648 | −1.2 | −1.73 | −1.13 | −1.65 |
| PLAU | NM_002658 | −1.74 | −1.95 | −1.77 | −1.91 |
| PLS3 | NM_005032 | −1.44 | −1.57 | −1.56 | −1.84 |
| POLQ | NM_006596 | −1.03 | −1.18 | −1.1 | −1.23 |
| POLS | NM_006999 | −1.23 | −1.39 | −1.17 | −1.38 |
| PPP1CA | NM_002708 | −1.83 | −2.18 | −1.76 | −2.42 |
| PRPF38A | NM_032864 | −1.11 | −1.17 | −1.09 | −1.17 |
| RACGAP1 | NM_013277 | −1.34 | −1.9 | −1.31 | −1.84 |
| RAD1 | NM_002853 | −1.2 | −1.37 | −1.12 | −1.38 |
| RAD21 | NM_006265 | | −1.36 | −1.32 | −1.52 |
| RAD51 | NM_002875 | 1.08 | −1.23 | 1.05 | −1.35 |
| RAP1GAP | NM_002885 | −1.11 | −1.43 | −1.21 | −1.35 |
| RB1 | NM_000321 | −1.21 | −1.45 | −1.25 | −1.39 |
| SEPT10 | NM_144710 | −1.59 | −1.98 | −1.6 | −1.95 |
| SERTAD2 | NM_014755 | −1.12 | −1.31 | −1.09 | −1.1 |
| SH3BP4 | NM_014521 | −1.52 | −1.9 | −1.38 | −1.67 |
| SMARCB1 | NM_003073 | −1.16 | −1.3 | −1.42 | −1.42 |
| TDG | NM_003211 | −1.47 | −1.52 | −1.54 | −1.55 |
| TDP1 | NM_018319 | −1.44 | −1.66 | −1.5 | −1.8 |
| TOP1 | NM_003286 | −1.52 | −1.66 | −1.64 | −1.76 |
| TUBGCP3 | NM_006322 | −1.26 | −1.52 | −1.41 | −1.73 |
| USP1 | NM_003368 | −1.13 | −1.35 | −1.16 | −1.38 |
| UVRAG | HSS00170585 | −1.2 | −1.38 | −1.17 | −1.43 |

¹The sequences of the Genbank reference numbers cited in TABLE 3 are each incorporated herein by reference.

Discussion

In this Example, it is demonstrated that genotoxic stress promotes the p53-dependent up-regulation of the homologous miRNAs, miR-192 and miR-215. Furthermore, a downstream gene expression signature for miR-192/miR-215 expression has been identified that includes a number of transcripts that regulate G₁ and G₂ checkpoints.

Similar to that observed for miR-34a, activation of miR-192/miR-215 induces cell cycle arrest, suggesting that multiple microRNA families operate in the p53 network. Furthermore, the gene expression signature identified in miR-192/miR-215 transfected cells includes a number of transcripts that regulate G₁ and G₂ checkpoints.

### Example 3

This Example demonstrates that the introduction of synthetic duplexes corresponding to miR-192 into HCT116DICER^{ex5} Cells delays cell cycle progression.

Rationale:

To test the hypothesis that miR-192/miR-215 function as cell cycle regulators directly, an experiment was carried out to examine the effect on cell cycle distribution of cells transfected with synthetic duplexes corresponding to miR-192. As shown in FIG. 1, miR-192 and miR-215 are highly homologous and have corresponding seed regions that are nearly identical. In view of the data described in Example 1 showing nearly identical transcriptional profiles post transfection, it was decided to focus on miR-192 for further studies, which is believed to be representative of miR-215.

Methods:

In this Example, a synthetic miR-192 duplex mimimetic (SEQ ID NO:1/SEQ ID NO:7) and a seed region mutant miR-192 duplex (SEQ ID NO:8/SEQ ID NO:9), as shown in TABLE 1, were transiently transfected into HCT116DICER^{ex5} cells at 10 nM final concentration using Lipofectamine RNAiMax (Invitrogen), according to the manufacturer's instructions. At 48 hours post transfection, cells were left untreated, treated with nocodazole (100 ng/mL, Sigma-Aldrich), or treated with aphidicolin (2 μg/mL, Sigma-Aldrich) for an additional 18 hours before harvesting. The cells were then trypsinized, collected, fixed and stained with a propidium iodide solution. BrdU-labeling was performed according to the manufacturer's instructions (BD-Pharmigen). For phospho-histone H3 analysis, cells were fixed and permeabilized with IPF buffer (100 mM PIPES pH 6.8, 10 mM EDTA, 1 mM MgCl₂, 0.2% Triton X-100, 4% formaldehyde) and stained with propidium iodide and anti-phospho-histone H3 antibody conjugated to Alexa-488 (Cell Signaling Technology). The cells were analyzed using a FACSCalibur flow cytometer (Beacton Dickinson) and FlowJo software (Tree Star, Inc.).

Results:

The cell cycle distribution of the HCT116DICER^{ex5} cells transfected with miR-192 or the miR-192 mutant are shown below in TABLE 4.

TABLE 4
Cell cycle distribution of HCT116DICER^{ex5} cells 66 hours
after transfection with either miR-192 siRNA synthetic duplex,
or with miR-192 mutant siRNA duplex

| | % Cells | % Cells |
|---|---|---|
| Treatment | with 2N | with 4N |
| conditions | (G1/S) | (G2/M) |
| untreated: miR-192 | 48.6% | 44.6% |
| untreated: miR-192 | 49.9% | 30.8% |
| mutant | | |
| nocodazole: miR-192 | 36.3% | 56.5% |
| nocodazole: miR-192 | 17% | 66.3% |
| mutant | | |
| aphidicholin: | 46.3% | 47.4% |
| miR-192 | | |
| aphidicholin: | 66.5% | 16.1% |
| miR-192 mutant | | |

As shown in TABLE 4, compared with mock (not shown) or mutant miR-192-transfected cells, wild type miR-192-transfected cells showed a significant decrease in S phase and an increase in G2/M phase populations. Similar effects on the cell cycle were observed in miR-215 transfected cells (data not shown).

To investigate further the miR-192 induced G1 arrest phenotype, the transfected cells were treated with the microtubule depolymerizing agent nocodazole, which traps cells at the G2/M phase, and reveals G1 arrest phenotypes (Linsley, P. S., et al., Mol. Cell Biol. 27:2240-2252 (2007)). As shown above in TABLE 4, in response to nocodazole, 17% of the miR-192 mutant duplex transfected cells remained in the G1 phase. In contrast, as further shown in TABLE 4, 36% of the miR-192 transfected cells accumulated in G1.

To address the G2/M arrest phenotype induced by miR-192, the transfected cells were treated with the DNA synthesis inhibitor aphidicolin, which causes cells to accumulate in G1 and reveals defects in cell cycle progression through G2/M phase. As shown in TABLE 4, when treated with aphidicolin, a significant fraction of miR-192 transfected cells accumulated in the G2/M phase, whereas miR-192 mutant transfected cells did not.

The transfected cells were also pulse-labeled with the thymidine analog 5-bromo-deoxyuridine (BrdU) to assay for defects in DNA synthesis. Using flow cytometry and it was determined that the percentage of cells in S-phase in the mock transfected population was 37.3%, whereas the percentage of cells in S-phase in the miR-192 transfected population was 11.9%. Taken together, these results indicate that expression of miR-192 prevented cells from transitioning from G1 to S phase.

Nocodazole-treated mock transfected and miR-192 transfected cells were also permeabilized, immunostained for phospho-histone-H3 (a mitotic marker) and sorted for DNA content and the respective accumulation of positively stained cells in the mitotic compartment was quantified. It was determined that the percentage of cells in M-phase in the mock transfected population was 40.9%, whereas the percentage of cells in M-phase in the miR-192 transfected population was 4.9%.

Discussion

In the current study, we have demonstrated that genotoxic stress promotes p53-dependent up-regulation of the miR-192/miR-215 family and that enforced expression of miR-192 or miR-215 leads to G₁ and G₂ cell cycle arrest. These results, together with the observation that miR-192 down-regulated transcripts are enriched for cell cycle related genes, leads to the conclusion that miR-192 and miR-215 function to delay cell cycle progression and act as tumor-suppressors.

It has long been observed that p53 activation leads to both induction and repression of transcripts (Zhao et al., Genes Dev. 14:981-993 (2000)). Compared with its well-studied transcriptional activation function, the p53 transcriptional repression function remains relatively uncharacterized. p53 can suppress gene expression via several potential mechanisms, including inhibition of activators, recruitment of co-repressors to target promoters and direct inhibition of the basal transcriptional machinery (Ho, J. S., et al., Mol. Cell Biol. 25:7423-7431 (2005); Ho, J., et al., Cell Death Differ. 10:404-408 (2003); Scian, J. J., et al., Oncogene 27:2583-2593 (2008); Tang, X., et al., Oncogene 23:5759-5769 (2004); St. Clair, S., et al., Mol. Cell 16:725-736 (2004)). Recently, miR-34a was established as a direct transcriptional target of p53 that contributes to p53 tumor suppressor function through down-regulating a number of target transcripts (He, L., et al., Nature 447:1130-1134 (2007)). As described in more detail in Examples 4-6, by simultaneously regulating the expression of key cell cycle genes, it is believed that miR-192 and miR-215 may mediate the cell cycle arrest function of p53.

### Example 4

This Example describes the transfection of siRNAs targeting miR-192/miR-215 responsive genes in HCT116DICER^{ex5} cells to identify direct downstream targets of miR-192/miR-215 that were downregulated in transfected cells.

Methods:

To identify targets whose modulation influences the cell cycle arrest phenotype observed with miR-192 expression, the 62 candidate genes identified as described in Example 2 and shown in TABLE 3 were silenced individually by transfecting a pool of 3 gene-specific siRNA duplexes per gene (each directed to a different region of the gene) into HCT116DICER^{ex5} cells. The siRNA duplex sequences (3 duplexes per gene) used to target each of the 62 genes are shown below in TABLE 5. The siRNA duplex oligonucleotides were obtained from Sigma-Genesys, as described in Linsley, P. S. et al., Mol Cell Biol 27:2240-2252 (2007).

HCT116DICER^{ex5} cells were transiently transfected with pools of three different siRNA duplexes per gene at a 100 nM final concentration using Lipofectamine RNAiMax (Invitrogen), according to the manufacturer's instructions. At 48 hours post transfection, cells were left untreated, treated with nocodazole (100 ng/mL, Sigma-Aldrich), or treated with aphidicolin (2 μg/mL, Sigma-Aldrich) for an additional 18 hours before harvesting. The cells transfected with the siRNA pools were then screened by FACS analysis as described in Example 3 for the percentage of cells arrested in G1 or G2/M as compared to the negative siRNA luciferase control.

The siRNA pools tested in this screen were ranked according to the percentage of cells arrested in G1 or G2/M following transfection, as compared with a negative control siRNA targeting luciferase. To avoid being misled by possible RNAi off-target effects, the siRNA pools were deconvoluted to identify genes for which at least 2 different siRNA duplexes from the pool caused the cell cycle arrest phenotype (data not shown).

TABLE 5
Synthetic miR-192 siRNA Oligonucleotide Sequences

| Identifier | Type | Guide Sequence | Passenger Sequence |
|---|---|---|---|
| miR-192 | micro-RNA | CUGACCUAUGAAUUGACAGCC | CUGUCAAUUCAUCGGUCUGAU |
| | | (SEQ ID NO: 1) | (SEQ ID NO: 7) |
| miR-192mut | micro-RNA | CUGCACUAUGAAUUGACAGCC | CUGUCAAUUCAUAGUGCUGAU |
| | | (SEQ ID NO: 8) | (SEQ ID NO: 9) |
| miR-215 | micro-RNA | AUGACCUAUGAAUUGACAGAC | CUGUCAAUUCAUAGGUCUUAU |
| | | (SEQ ID NO: 4) | (SEQ ID NO: 10) |
| BCL2-1 | siRNA | UCAAAGAAGGCCACAAUCCTT | GGAUUGUGGCCUUCUUUGATT |
| | | (SEQ ID NO: 103) | (SEQ ID NO: 104) |
| BCL2-2 | siRNA | UUGUGGCCCAGAUAGGCACTT | GUGCCUAUCUGGGCCACAATT |
| | | (SEQ ID NO: 105) | (SEQ ID NO: 106) |
| BCL2-3 | siRNA | AUGCAAGUGAAUGAACACCTT | GGUGUUCAUUCACUUGCAUTT |
| | | (SEQ ID NO: 107) | (SEQ ID NO: 108) |
| BRCA1-1 | siRNA | UUGCAUGGAAGCCAUUGUCTT | GACAAUGGCUUCCAUGCAATT |
| | | (SEQ ID NO: 121) | (SEQ ID NO: 122) |
| BRCA1-2 | siRNA | UUAGUAGCCAGGACAGUAGTT | CUACUGUCCUGGCUACUAATT |
| | | (SEQ ID NO: 123) | (SEQ ID NO: 124) |
| BRCA1-3 | siRNA | UGAAUAGAAAGAAUAGGGCTT | GCCCUAUUCUUUCUAUUCATT |
| | | (SEQ ID NO: 125) | (SEQ ID NO: 126) |
| CDC14A-1 | siRNA | ACAUAACAGGCUAUCAAUGTT | CAUUGAUAGCCUGUUAUGUTT |
| | | (SEQ ID NO: 127) | (SEQ ID NO: 128) |
| CDC14A-2 | siRNA | UUGUUUAGCCUCACAACUGTT | CAGUUGUGAGGCUAAACAATT |
| | | (SEQ ID NO: 129) | (SEQ ID NO: 130) |
| CDC14A-3 | siRNA | AUAGUGGGUAUUUACUGUGTT | CACAGUAAAUACCCACUAUTT |
| | | (SEQ ID NO: 131) | (SEQ ID NO: 132) |
| CDC7-1 | siRNA | AACUACAUGAUCAUUCUUCTT | GAAGAAUGAUCAUGUAGUUTT |
| | | (SEQ ID NO: 55) | (SEQ ID NO: 56) |
| CDC7-2 | siRNA | UCCCAUGACAUUAUCUUGCTT | GCAAGAUAAUGUCAUGGGATT |
| | | (SEQ ID NO: 57) | (SEQ ID NO: 58) |
| CDC7-3 | siRNA | AGUACAUCCACAGUCUUUGTT | CAAAGACUGUGGAUGUACUTT |
| | | (SEQ ID NO: 59) | (SEQ ID NO: 60) |
| CDC73-1 | siRNA | AAAGACCUAAUCUGUUCAGTT | CUGAACAGAUUAGGUCUUUTT |
| | | (SEQ ID NO: 133) | (SEQ ID NO: 134) |
| CDC73-2 | siRNA | UUCGAUCAGGUCUUCUAACTT | GUUAGAAGACCUGAUCGAATT |
| | | (SEQ ID NO: 135) | (SEQ ID NO: 136) |
| CDC73-3 | siRNA | UCUUCAUCCUCAAUUCGUGTT | CACGAAUUGAGGAUGAAGATT |
| | | (SEQ ID NO: 137) | (SEQ ID NO: 138) |
| CRK-1 | siRNA | UCUUCGUAACCUUUACCAGTT | CUGGUAAAGGUUACGAAGATT |
| | | (SEQ ID NO: 139) | (SEQ ID NO: 140) |
| CRK-2 | siRNA | AUGCUUAUAUAAACUAGACTT | GUCUAGUUUAUAUAAGCAUTT |
| | | (SEQ ID NO: 141) | (SEQ ID NO: 142) |
| CRK-3 | siRNA | AAUCAGAGCCGAUACUGAGTT | CUCAGUAUCGGCUCUGAUUTT |
| | | (SEQ ID NO: 143) | (SEQ ID NO: 144) |
| CTCF-1 | siRNA | UUGCCUUGCUCAAUAUAGGTT | CCUAUAUUGAGCAAGGCAATT |
| | | (SEQ ID NO: 145) | (SEQ ID NO: 146) |
| CTCF-2 | siRNA | UACCACUUUGGGUAAACCGTT | CGGUUUACCCAAAGUGGUATT |
| | | (SEQ ID NO: 147) | (SEQ ID NO: 148) |
| CTCF-3 | siRNA | UGGCGGAAGGUCUUAUCGCTT | GCGAUAAGACCUUCCGCCATT |
| | | (SEQ ID NO: 149) | (SEQ ID NO: 150) |
| CUL5-1 | siRNA | UACUAAAGACCAUAAAGUCTT | GACUUUAUGGUCUUUAGUATT |
| | | (SEQ ID NO: 109) | (SEQ ID NO: 110) |
| CUL5-2 | siRNA | UACCAUUAGGAACUUUGUCTT | GACAAAGUUCCUAAUGGUATT |
| | | (SEQ ID NO: 111) | (SEQ ID NO: 112) |
| CUL5-3 | siRNA | AGAUGUCUAAUAUAAGACGTT | CGUCUUAUAUUAGACAUCUTT |
| | | (SEQ ID NO: 113) | (SEQ ID NO: 114) |
| DBF4B-1 | siRNA | AAUCGUCUCCCUUUCCCGGTT | CCGGGAAAGGGAGACGAUUTT |
| | | (SEQ ID NO: 151) | (SEQ ID NO: 152) |
| DBF4B-2 | siRNA | GAACUCUCCAGCUCGAGGCTT | GCCUCGAGCUGGAGAGUUCTT |
| | | (SEQ ID NO: 153) | (SEQ ID NO: 154) |
| DBF4B-3 | siRNA | ACACCUGGAAACUCCUAGGTT | CCUAGGAGUUUCCAGGUGUTT |
| | | (SEQ ID NO: 155) | (SEQ ID NO: 156) |
| DEK-1 | siRNA | AUGGGAACGAGUCAUCUUCTT | GAAGAUGACUCGUUCCCAUTT |
| | | (SEQ ID NO: 157) | (SEQ ID NO: 158) |
| DEK-2 | siRNA | ACUAGUUCACUAUUUACACTT | GUGUAAAUAGUGAACUAGUTT |
| | | (SEQ ID NO: 159) | (SEQ ID NO: 160) |
| DEK-3 | siRNA | AUGGAAAGCCACUGAACUGTT | CAGUUCAGUGGCUUUCCAUTT |
| | | (SEQ ID NO: 161) | (SEQ ID NO: 162) |
| DLG5-1 | siRNA | UAGCGUGCGGAGCAAUGUCTT | GACAUUGCUCCGCACGCUATT |
| | | (SEQ ID NO: 97) | (SEQ ID NO: 98) |
| DLG5-2 | siRNA | UGCGUUCCGCCUUGAACUGTT | CAGUUCAAGGCGGAACGCATT |
| | | (SEQ ID NO: 99) | (SEQ ID NO: 100) |
| DLG5-3 | siRNA | UUCAUUCAGAGACUUGUUGTT | CAACAAGUCUCUGAAUGAATT |
| | | (SEQ ID NO: 101) | (SEQ ID NO: 102) |
| DTL-1 | siRNA | UAUAAUUCUUACGUAAAUCTT | GAUUUACGUAAGAAUUAUATT |
| | | (SEQ ID NO: 73) | (SEQ ID NO: 74) |
| DTL-2 | siRNA | UCUAUAAUUCUGUUGAGUGTT | CACUCAACAGAAUUAUAGATT |
| | | (SEQ ID NO: 75) | (SEQ ID NO: 76) |
| DTL-3 | siRNA | UCGAUAAGCAGUAUAAUUCTT | GAAUUAUACUGCUUAUCGATT |
| | | (SEQ ID NO: 77) | (SEQ ID NO: 78) |
| EMP1-1 | siRNA | AAUAGCAUAAUAACAGUAGTT | CUACUGUUAUUAUGCUAUUTT |
| | | (SEQ ID NO: 163) | (SEQ ID NO: 164) |
| EMP1-2 | siRNA | UAAUGACUAGUGUAGAUGGTT | CCAUCUACACUAGUCAUUATT |
| | | (SEQ ID NO: 165) | (SEQ ID NO: 166) |
| EMP1-3 | siRNA | UAGCAUAAUAACAGUAGCGTT | CGCUACUGUUAUUAUGCUATT |
| | | (SEQ ID NO: 167) | (SEQ ID NO: 168) |
| ERCC3-1 | siRNA | UAGGUCCGUAGAUAUAGGGTT | CCCUAUAUCUACGGACCUATT |
| | | (SEQ ID NO: 37) | (SEQ ID NO: 38) |
| ERCC3-2 | siRNA | UUCUUAAGCGGCAUUCUCGTT | CGAGAAUGCCGCUUAAGAATT |
| | | (SEQ ID NO: 39) | (SEQ ID NO: 40) |
| ERCC3-3 | siRNA | UCAAACCCAGCUUACAGUGTT | CACUGUAAGCUGGGUUUGATT |
| | | (SEQ ID NO: 41) | (SEQ ID NO: 42) |
| EXTL2-1 | siRNA | UACGUCUGCAUUAUGAGAGTT | CUCUCAUAAUGCAGACGUATT |
| | | (SEQ ID NO: 169) | (SEQ ID NO: 170) |
| EXTL2-2 | siRNA | UAAUCGAAGCACUCGAAUCTT | GAUUCGAGUGCUUCGAUUATT |
| | | (SEQ ID NO: 171) | (SEQ ID NO: 172) |
| EXTL2-3 | siRNA | ACGAGGAUGACCACCAAAGTT | CUUUGGUGGUCAUCCUCGUTT |
| | | (SEQ ID NO: 173) | (SEQ ID NO: 174) |
| FGF2-1 | siRNA | UCCAAACUGAGCUAUACACTT | GUGUAUAGCUCAGUUUGGATT |
| | | (SEQ ID NO: 175) | (SEQ ID NO: 176) |
| FGF2-2 | siRNA | UGACCAAUUAUCCAAACUGTT | CAGUUUGGAUAAUUGGUCATT |
| | | (SEQ ID NO: 177) | (SEQ ID NO: 178) |
| FGF2-3 | siRNA | UCAUGUGAAAUGAGAUUAGTT | CUAAUCUCAUUUCACAUGATT |
| | | (SEQ ID NO: 179) | (SEQ ID NO: 180) |
| HOXA10-1 | siRNA | UAACGGCCCAGGAGAUGGCTT | GCCAUCUCCUGGGCCGUUATT |
| | | (SEQ ID NO: 31) | (SEQ ID NO: 32) |
| HOXA10-2 | siRNA | AAAUAAACCAGCACCAAGCTT | GCUUGGUGCUGGUUUAUUUTT |
| | | (SEQ ID NO: 33) | (SEQ ID NO: 34) |
| HOXA10-3 | siRNA | ACAGGUGCGAGUUCCUGGGTT | CCCAGGAACUCGCACCUGUTT |
| | | (SEQ ID NO: 35) | (SEQ ID NO: 36) |
| HRH1-1 | siRNA | UCCCUUAGGAGCGAAUAUGTT | CAUAUUCGCUCCUAAGGGATT |
| | | (SEQ ID NO: 25) | (SEQ ID NO: 26) |
| HRH1-2 | siRNA | UAAUCCAGGCCUGUGUUAGTT | CUAACACAGGCCUGGAUUATT |
| | | (SEQ ID NO: 27) | (SEQ ID NO: 28) |
| HRH1-3 | siRNA | UUGGCUAUCACCUAACAUCTT | GAUGUUAGGUGAUAGCCAATT |
| | | (SEQ ID NO: 29) | (SEQ ID NO: 30) |
| KIF14-1 | siRNA | UCUAUUAGGUUAAUUCGACTT | GUCGAAUUAACCUAAUAGATT |
| | | (SEQ ID NO: 181) | (SEQ ID NO: 182) |
| KIF14-2 | siRNA | UCCCGCUUGAUUUAGAUUGTT | CAAUCUAAAUCAAGCGGGATT |
| | | (SEQ ID NO: 183) | (SEQ ID NO: 184) |
| KIF14-3 | siRNA | ACGAACCCGAAUAGAAGUGTT | CACUUCUAUUCGGGUUCGUTT |
| | | (SEQ ID NO: 185) | (SEQ ID NO: 186) |
| KIF23-1 | siRNA | UUUCGACUACCAUUUGGUGTT | CACCAAAUGGUAGUCGAAATT |
| | | (SEQ ID NO: 187) | (SEQ ID NO: 188) |
| KIF23-2 | siRNA | AAUUAGUUUAGUUUCAAUCTT | GAUUGAAACUAAACUAAUUTT |
| | | (SEQ ID NO: 189) | (SEQ ID NO: 190) |
| KIF23-3 | siRNA | UCGAUAUGGAACCAUCUUGTT | CAAGAUGGUUCCAUAUCGATT |
| | | (SEQ ID NO: 191) | (SEQ ID NO: 192) |
| LMNB2-1 | siRNA | AUCUUCCGGAACUUGUCCCTT | GGGACAAGUUCCGGAAGAUTT |
| | | (SEQ ID NO: 19) | (SEQ ID NO: 20) |
| LMNB2-2 | siRNA | UUCCCGCUGUCCGAAGCUGTT | CAGCUUCGGACAGCGGGAATT |
| | | (SEQ ID NO: 21) | (SEQ ID NO: 22) |
| LMNB2-3 | siRNA | UGGGCGUGAACUUGUAGGCTT | GCCUACAAGUUCACGCCCATT |
| | | (SEQ ID NO: 23) | (SEQ ID NO: 24) |
| MACF1-1 | siRNA | UCCAUCGAGCAUUGAUCUCTT | GAGAUCAAUGCUCGAUGGATT |
| | | (SEQ ID NO: 193) | (SEQ ID NO: 194) |
| MACF1-2 | siRNA | UAAUUGUGGUAUCAUACACTT | GUGUAUGAUACCACAAUUATT |
| | | (SEQ ID NO: 195) | (SEQ ID NO: 196) |
| MACF1-3 | siRNA | UCAAGUAGUUGAUUAUAAGTT | CUUAUAAUCAACUACUUGATT |
| | | (SEQ ID NO: 197) | (SEQ ID NO: 198) |
| MAD2L1-1 | siRNA | UAGUAGUAAAUGAACGAAGTT | CUUCGUUCAUUUACUACUATT |
| | | (SEQ ID NO: 67) | (SEQ ID NO: 68) |
| MAD2L1-2 | siRNA | UGAACAAGAAACUUCCAACTT | GUUGGAAGUUUCUUGUUCATT |
| | | (SEQ ID NO: 69) | (SEQ ID NO: 70) |
| MAD2L1-3 | siRNA | UCACCGUAGCUGUGAUCUGTT | CAGAUCACAGCUACGGUGATT |
| | | (SEQ ID NO: 71) | (SEQ ID NO: 72) |
| MCM10-1 | siRNA | ACGAAGAUCAUUCAGUUUCTT | GAAACUGAAUGAUCUUCGUTT |
| | | (SEQ ID NO: 85) | (SEQ ID NO: 86) |
| MCM10-2 | siRNA | AUGUCACCCAAUCUAUUUCTT | GAAAUAGAUUGGGUGACAUTT |
| | | (SEQ ID NO: 87) | (SEQ ID NO: 88) |
| MCM10-3 | siRNA | AUCCCACUAGGUUUGUUCCTT | GGAACAAACCUAGUGGGAUTT |
| | | (SEQ ID NO: 89) | (SEQ ID NO: 90) |
| MCM3-1 | siRNA | AGAAUAACGUCGCUCUAUGTT | CAUAGAGCGACGUUAUUCUTT |
| | | (SEQ ID NO: 199) | (SEQ ID NO: 200) |
| MCM3-2 | siRNA | AACUAGAGAACAUUUAGUGTT | CACUAAAUGUUCUCUAGUUTT |
| | | (SEQ ID NO: 201) | (SEQ ID NO: 202) |
| MCM3-3 | siRNA | AACAUUACAGGCAAUCAGGTT | CCUGAUUGCCUGUAAUGUUTT |
| | | (SEQ ID NO: 203) | (SEQ ID NO: 204) |
| MCM6-1 | siRNA | AUAGAACUCCUCUUGAAUGTT | CAUUCAAGAGGAGUUCUAUTT |
| | | (SEQ ID NO: 205) | (SEQ ID NO: 206) |
| MCM6-2 | siRNA | UGAAUGCAAAUCUACUAUGTT | CAUAGUAGAUUUGCAUUCATT |
| | | (SEQ ID NO: 207) | (SEQ ID NO: 208) |
| MCM6-3 | siRNA | UCAGCCAACAUCAAAGCUCTT | GAGCUUUGAUGUUGGCUGATT |
| | | (SEQ ID NO: 209) | (SEQ ID NO: 210) |
| MIS12-1 | siRNA | UGAACAGUCCUUAUGAUUCTT | GAAUCAUAAGGACUGUUCATT |
| | | (SEQ ID NO: 43) | (SEQ ID NO: 44) |
| MIS12-2 | siRNA | UCACUAGUCCCAUGAUCUCTT | GAGAUCAUGGGACUAGUGATT |
| | | (SEQ ID NO: 45) | (SEQ ID NO: 46) |
| MIS12-3 | siRNA | UGACUACUGAGCAAUUAAGTT | CUUAAUUGCUCAGUAGUCATT |
| | | (SEQ ID NO: 47) | (SEQ ID NO: 48) |
| MKI67-1 | siRNA | AAUACACUGCCGUCUUAAGTT | CUUAAGACGGCAGUGUAUUTT |
| | | (SEQ ID NO: 211) | (SEQ ID NO: 212) |
| MKI67-2 | siRNA | UCCCUAAACGCGUUGAUGCTT | GCAUCAACGCGUUUAGGGATT |
| | | (SEQ ID NO: 213) | (SEQ ID NO: 214) |
| MKI67-3 | siRNA | UGAAAUUAUGUAAUAUUGCTT | GCAAUAUUACAUAAUUUCATT |
| | | (SEQ ID NO: 215) | (SEQ ID NO: 216) |
| MPHOSPH1- | siRNA | UUUGAUUAAACUUUAGUUCTT | GAACUAAAGUUUAAUCAAATT |
| 1 | | (SEQ ID NO: 49) | (SEQ ID NO: 50) |
| MPHOSPH1- | siRNA | UCUGAAAGCGCUCUCUUUCTT | GAAAGAGAGCGCUUUCAGATT |
| 2 | | (SEQ ID NO: 51) | (SEQ ID NO: 52) |
| MPHOSPH1- | siRNA | AACUUCCAUAAAGAGUAUCTT | GAUACUCUUUAUGGAAGUUTT |
| 3 | | (SEQ ID NO: 53) | (SEQ ID NO: 54) |
| MTSS1-1 | siRNA | UUCCCAAACUGGAUAGCUCTT | GAGCUAUCCAGUUUGGGAATT |
| | | (SEQ ID NO: 217) | (SEQ ID NO: 218) |
| MTSS1-2 | siRNA | AAUCAGAACCUUUCAAGUCTT | GACUUGAAAGGUUCUGAUUTT |
| | | (SEQ ID NO: 219) | (SEQ ID NO: 220) |
| MTSS1-3 | siRNA | UGAGAUUUCUUCUUCAAUCTT | GAUUGAAGAAGAAAUCUCATT |
| | | (SEQ ID NO: 221) | (SEQ ID NO: 222) |
| NBN-1 | siRNA | UAUGCCAGAUGGAUUUCUGTT | CAGAAAUCCAUCUGGCAUATT |
| | | (SEQ ID NO: 223) | (SEQ ID NO: 224) |
| NBN-2 | siRNA | UUAGCCACUCUUCUAGUUCTT | GAACUAGAAGAGUGGCUAATT |
| | | (SEQ ID NO: 225) | (SEQ ID NO: 226) |
| NBN-3 | siRNA | UUAACACAGCAUGAUUUCGTT | CGAAAUCAUGCUGUGUUAATT |
| | | (SEQ ID NO: 227) | (SEQ ID NO: 228) |
| NCAPH-1 | siRNA | UUGCGUCGAGGCCUAAAGCTT | GCUUUAGGCCUCGACGCAATT |
| | | (SEQ ID NO: 229) | (SEQ ID NO: 230) |
| NCAPH-2 | siRNA | AUGUUGUGAUGUCUAAUCCTT | GGAUUAGACAUCACAACAUTT |
| | | (SEQ ID NO: 231) | (SEQ ID NO: 232) |
| NCAPH-3 | siRNA | AAUAGUUCUGUGUAAGUGCTT | GCACUUACACAGAACUAUUTT |
| | | (SEQ ID NO: 233) | (SEQ ID NO: 234) |
| NEK1-1 | siRNA | UUCCACCAGCACUUAGCACTT | GUGCUAAGUGCUGGUGGAATT |
| | | (SEQ ID NO: 235) | (SEQ ID NO: 236) |
| NEK1-2 | siRNA | UUAGACACCGCCUUCAAUCTT | GAUUGAAGGCGGUGUCUAATT |
| | | (SEQ ID NO: 237) | (SEQ ID NO: 238) |
| NEK1-3 | siRNA | ACUAAAUGAAGAAUCUUGGTT | CCAAGAUUCUUCAUUUAGUTT |
| | | (SEQ ID NO: 239) | (SEQ ID NO: 240) |
| NFIB-1 | siRNA | AAAGUCCUCUCGAUACUCCTT | GGAGUAUCGAGAGGACUUUTT |
| | | (SEQ ID NO: 241) | (SEQ ID NO: 242) |
| NFIB-2 | siRNA | UCACGGUGAGCACAAAGUCTT | GACUUUGUGCUCACCGUGATT |
| | | (SEQ ID NO: 243) | (SEQ ID NO: 244) |
| NFIB-3 | siRNA | AGACGCCAGACUUUGUCUGTT | CAGACAAAGUCUGGCGUCUTT |
| | | (SEQ ID NO: 245) | (SEQ ID NO: 246) |
| NFYA-1 | siRNA | UGUCAUUGCUUCUUCAUCGTT | CGAUGAAGAAGCAAUGACATT |
| | | (SEQ ID NO: 247) | (SEQ ID NO: 248) |
| NFYA-2 | siRNA | AGGACACUCGGAUGAUCUGTT | CAGAUCAUCCGAGUGUCCUTT |
| | | (SEQ ID NO: 249) | (SEQ ID NO: 250) |
| NFYA-3 | siRNA | UCUGUCCUGUAGUAAAGGGTT | CCCUUUACUACAGGACAGATT |
| | | (SEQ ID NO: 251) | (SEQ ID NO: 252) |
| NIN-1 | siRNA | UAAGGUGUGCGUUUCGUUCTT | GAACGAAACGCACACCUUATT |
| | | (SEQ ID NO: 253) | (SEQ ID NO: 254) |
| NIN-2 | siRNA | ACAGUCCGCACAUAACAUCTT | GAUGUUAUGUGCGGACUGUTT |
| | | (SEQ ID NO: 255) | (SEQ ID NO: 256) |
| NIN-3 | siRNA | UCCAUCGAGGCUGAAAUCCTT | GGAUUUCAGCCUCGAUGGATT |
| | | (SEQ ID NO: 257) | (SEQ ID NO: 258) |
| PAFAH1B1-1 | siRNA | AUCUUAAUAGUCUUGUCUCTT | GAGACAAGACUAUUAAGAUTT |
| | | (SEQ ID NO: 259) | (SEQ ID NO: 260) |
| PAFAH1B1-2 | siRNA | UUGCUACGACCCAUACACGTT | CGUGUAUGGGUCGUAGCAATT |
| | | (SEQ ID NO: 261) | (SEQ ID NO: 262) |
| PAFAH1B1-3 | siRNA | AUUUAAACAGAGCUCAAUGTT | CAUUGAGCUCUGUUUAAAUTT |
| | | (SEQ ID NO: 263) | (SEQ ID NO: 264) |
| PAPD5-1 | siRNA | UUACUCUAAUUAUUCUACCTT | GGUAGAAUAAUUAGAGUAATT |
| | | (SEQ ID NO: 265) | (SEQ ID NO: 266) |
| PAPD5-2 | siRNA | UGAACCACCAUCCUUUAUCTT | GAUAAAGGAUGGUGGUUCATT |
| | | (SEQ ID NO: 267) | (SEQ ID NO: 268) |
| PAPD5-3 | siRNA | UUUGCUAUUGGUGAUACAGTT | CUGUAUCACCAAUAGCAAATT |
| | | (SEQ ID NO: 269) | (SEQ ID NO: 270) |
| PFTK1-1 | siRNA | UAACGCUGGUGGAUGUAAGTT | CUUACAUCCACCAGCGUUATT |
| | | (SEQ ID NO: 271) | (SEQ ID NO: 272) |
| PFTK1-2 | siRNA | UAGCUGAGCUUAUUCCAUGTT | CAUGGAAUAAGCUCAGCUATT |
| | | (SEQ ID NO: 273) | (SEQ ID NO: 274) |
| PFTK1-3 | siRNA | AAAGUUAAUGCAAGCAUUGTT | CAAUGCUUGCAUUAACUUUTT |
| | | (SEQ ID NO: 275) | (SEQ ID NO: 276) |
| PIM1-1 | siRNA | UCGGGUCCCAUCGAAGUCCTT | GGACUUCGAUGGGACCCGATT |
| | | (SEQ ID NO: 91) | (SEQ ID NO: 92) |
| PIM1-2 | siRNA | UGCUCGAAAGGAAUAUCUCTT | GAGAUAUUCCUUUCGAGCATT |
| | | (SEQ ID NO: 93) | (SEQ ID NO: 94) |
| PIM1-3 | siRNA | AGCAGGACCACUUCCAUGGTT | CCAUGGAAGUGGUCCUGCUTT |
| | | (SEQ ID NO: 95) | (SEQ ID NO: 96) |
| PLAU-1 | siRNA | UUGGACAAGCGGCUUUAGGTT | CCUAAAGCCGCUUGUCCAATT |
| | | (SEQ ID NO: 277) | (SEQ ID NO: 278) |
| PLAU-2 | siRNA | UGGACAAGCGGCUUUAGGCTT | GCCUAAAGCCGCUUGUCCATT |
| | | (SEQ ID NO: 279) | (SEQ ID NO: 280) |
| PLAU-3 | siRNA | UUGGAGAAGUACUUGUUGGTT | CCAACAAGUACUUCUCCAATT |
| | | (SEQ ID NO: 281) | (SEQ ID NO: 282) |
| PLS3-1 | siRNA | UUUAUAUCCUGGUAAUGGCTT | GCCAUUACCAGGAUAUAAATT |
| | | (SEQ ID NO: 283) | (SEQ ID NO: 284) |
| PLS3-2 | siRNA | AGACUAAAGCUAAAGUCAGTT | CUGACUUUAGCUUUAGUCUTT |
| | | (SEQ ID NO: 285) | (SEQ ID NO: 286) |
| PLS3-3 | siRNA | UAAUUCAACAGCAUAGUUGTT | CAACUAUGCUGUUGAAUUATT |
| | | (SEQ ID NO: 287) | (SEQ ID NO: 288) |
| POLQ-1 | siRNA | UUGUCUUUGAACCCAUUUCTT | GAAAUGGGUUCAAAGACAATT |
| | | (SEQ ID NO: 289) | (SEQ ID NO: 290) |
| POLQ-2 | siRNA | UAAUUCUGCCACAAGAGUCTT | GACUCUUGUGGCAGAAUUATT |
| | | (SEQ ID NO: 291) | (SEQ ID NO: 292) |
| POLQ-3 | siRNA | ACUGAGAGGGCAUUUCCACTT | GUGGAAAUGCCCUCUCAGUTT |
| | | (SEQ ID NO: 293) | (SEQ ID NO: 294) |
| POLS-1 | siRNA | UCUUCCUAAAGUACUUUCGTT | CGAAAGUACUUUAGGAAGATT |
| | | (SEQ ID NO: 295) | (SEQ ID NO: 296) |
| POLS-2 | siRNA | UGAGCUUUAUUAUUGGUACTT | GUACCAAUAAUAAAGCUCATT |
| | | (SEQ ID NO: 297) | (SEQ ID NO: 298) |
| POLS-3 | siRNA | UUCUACACCAGCUUGUCUGTT | CAGACAAGCUGGUGUAGAATT |
| | | (SEQ ID NO: 299) | (SEQ ID NO: 300) |
| PPP1CA-1 | siRNA | AAACUCGCCACAGUAGUUGTT | CAACUACUGUGGCGAGUUUTT |
| | | (SEQ ID NO: 301) | (SEQ ID NO: 302) |
| PPP1CA-2 | siRNA | AUCGUAGAAACCAUAGAUGTT | CAUCUAUGGUUUCUACGAUTT |
| | | (SEQ ID NO: 303) | (SEQ ID NO: 304) |
| PPP1CA-3 | siRNA | AGUUUGAUGUUGUAGCGUCTT | GACGCUACAACAUCAAACUTT |
| | | (SEQ ID NO: 305) | (SEQ ID NO: 306) |
| PRPF38A-1 | siRNA | UGACUACGGUGAUGACCUGTT | CAGGUCAUCACCGUAGUCATT |
| | | (SEQ ID NO: 115) | (SEQ ID NO: 116) |
| PRPF38A-2 | siRNA | UUGUGGCUCUUCUUAGACCTT | GGUCUAAGAAGAGCCACAATT |
| | | (SEQ ID NO: 117) | (SEQ ID NO: 118) |
| PRPF38A-3 | siRNA | GACCUUUCGGGAGACUUUGTT | CAAAGUCUCCCGAAAGGUCTT |
| | | (SEQ ID NO: 119) | (SEQ ID NO: 120) |
| RACGAP1-1 | siRNA | UCACACAUGAGCAUCUCUCTT | GAGAGAUGCUCAUGUGUGATT |
| | | (SEQ ID NO: 79) | (SEQ ID NO: 80) |
| RACGAP1-2 | siRNA | UUUACGGAAAUCCUCAAAGTT | CUUUGAGGAUUUCCGUAAATT |
| | | (SEQ ID NO: 81) | (SEQ ID NO: 82) |
| RACGAP1-3 | siRNA | UGUCACUGGGUCUGGAUUGTT | CAAUCCAGACCCAGUGACATT |
| | | (SEQ ID NO: 83) | (SEQ ID NO: 84) |
| RAD1-1 | siRNA | AAUAAGAACUUCAUCUAUCTT | GAUAGAUGAAGUUCUUAUUTT |
| | | (SEQ ID NO: 307) | (SEQ ID NO: 308) |
| RAD1-2 | siRNA | ACAAGAUAGGACUAAUGCCTT | GGCAUUAGUCCUAUCUUGUTT |
| | | (SEQ ID NO: 309) | (SEQ ID NO: 310) |
| RAD1-3 | siRNA | AAAGUCCCUGGCAUAGGACTT | GUCCUAUGCCAGGGACUUUTT |
| | | (SEQ ID NO: 311) | (SEQ ID NO: 312) |
| RAD51-1 | siRNA | UGAGCUACCACCUGAUUAGTT | CUAAUCAGGUGGUAGCUCATT |
| | | (SEQ ID NO: 313) | (SEQ ID NO: 314) |
| RAD51-2 | siRNA | AAGAUUGUCCAGUAGACAGTT | CUGUCUACUGGACAAUCUUTT |
| | | (SEQ ID NO: 315) | (SEQ ID NO: 316) |
| RAD51-3 | siRNA | UGAUAUUUCCUCCAAUAGGTT | CCUAUUGGAGGAAAUAUCATT |
| | | (SEQ ID NO: 317) | (SEQ ID NO: 318) |
| RAP1GAP-1 | siRNA | AACAUGAUCUCCUUGUUGCTT | GCAACAAGGAGAUCAUGUUTT |
| | | (SEQ ID NO: 319) | (SEQ ID NO: 320) |
| RAP1GAP-2 | siRNA | UAAAGUCCUGCAGUUUGACTT | GUCAAACUGCAGGACUUUATT |
| | | (SEQ ID NO: 321) | (SEQ ID NO: 322) |
| RAP1GAP-3 | siRNA | UCAAGGAACUCCACGAAAGTT | CUUUCGUGGAGUUCCUUGATT |
| | | (SEQ ID NO: 323) | (SEQ ID NO: 324) |
| RB1-1 | siRNA | UUCGAGUAGAAGUCAUUUCTT | GAAAUGACUUCUACUCGAATT |
| | | (SEQ ID NO: 325) | (SEQ ID NO: 326) |
| RB1-2 | siRNA | UCCGUAAGGGUGAACUAGGTT | CCUAGUUCACCCUUACGGATT |
| | | (SEQ ID NO: 327) | (SEQ ID NO: 328) |
| RB1-3 | siRNA | AUUUAUGGACACUGAUUUCTT | GAAAUCAGUGUCCAUAAAUTT |
| | | (SEQ ID NO: 329) | (SEQ ID NO: 330) |
| SEPT10-1 | siRNA | AUGGAUGCGAGAAUCAUGGTT | CCAUGAUUCUCGCAUCCAUTT |
| | | (SEQ ID NO: 13) | (SEQ ID NO: 14) |
| SEPT10-2 | siRNA | UACCUUGCUGUCAAGGUUCTT | GAACCUUGACAGCAAGGUATT |
| | | (SEQ ID NO: 15) | (SEQ ID NO: 16) |
| SEPT10-3 | siRNA | UAUCUCGGAGGUAGCUUUCTT | GAAAGCUACCUCCGAGAUATT |
| | | (SEQ ID NO: 17) | (SEQ ID NO: 18) |
| SERTAD2-1 | siRNA | UCCUAUACCAGGGUAGUAGTT | CUACUACCCUGGUAUAGGATT |
| | | (SEQ ID NO: 331) | (SEQ ID NO: 332) |
| SERTAD2-2 | siRNA | UAAAUGCAACACUUACGAGTT | CUCGUAAGUGUUGCAUUUATT |
| | | (SEQ ID NO: 333) | (SEQ ID NO: 334) |
| SERTAD2-3 | siRNA | ACGAGGAGUAGUUAAUGCCTT | GGCAUUAACUACUCCUCGUTT |
| | | (SEQ ID NO: 335) | (SEQ ID NO: 336) |
| SH3BP4-1 | siRNA | AUCGCAAUCACUUCCUUUGTT | CAAAGGAAGUGAUUGCGAUTT |
| | | (SEQ ID NO: 337) | (SEQ ID NO: 338) |
| SH3BP4-2 | siRNA | UGUUUAGGGCCGUAGAGACTT | GUCUCUACGGCCCUAAACATT |
| | | (SEQ ID NO: 339) | (SEQ ID NO: 340) |
| SH3BP4-3 | siRNA | UUCCAGAAAGGGUUAUUACTT | GUAAUAACCCUUUCUGGAATT |
| | | (SEQ ID NO: 341) | (SEQ ID NO: 342) |
| SMARCB1-1 | siRNA | AUGAUGACGCGCUGGUCUGTT | CAGACCAGCGCGUCAUCAUTT |
| | | (SEQ ID NO: 61) | (SEQ ID NO: 62) |
| SMARCB1-2 | siRNA | ACAUGUCCCACUCAAACUGTT | CAGUUUGAGUGGGACAUGUTT |
| | | (SEQ ID NO: 63) | (SEQ ID NO: 64) |
| SMARCB1-3 | siRNA | UUCAAAUCCAGAUCGUCACTT | GUGACGAUCUGGAUUUGAATT |
| | | (SEQ ID NO: 65) | (SEQ ID NO: 66) |
| TDG-1 | siRNA | AGAAGCACCAUUCUUAAGCTT | GCUUAAGAAUGGUGCUUCUTT |
| | | (SEQ ID NO: 343) | (SEQ ID NO: 344) |
| TDG-2 | siRNA | UCAACUGAUCUCUUAAGUCTT | GACUUAAGAGAUCAGUUGATT |
| | | (SEQ ID NO: 345) | (SEQ ID NO: 346) |
| TDG-3 | siRNA | AAAUCCAAUACCAUACUUCTT | GAAGUAUGGUAUUGGAUUUTT |
| | | (SEQ ID NO: 347) | (SEQ ID NO: 348) |
| TDP1-1 | siRNA | UAACCACUUUGAUUCAUCGTT | CGAUGAAUCAAAGUGGUUATT |
| | | (SEQ ID NO: 349) | (SEQ ID NO: 350) |
| TDP1-2 | siRNA | AUAUAUGUCUUAAUAUGUGTT | CACAUAUUAAGACAUAUAUTT |
| | | (SEQ ID NO: 351) | (SEQ ID NO: 352) |
| TDP1-3 | siRNA | UUAAACUCAGAACAUAACCTT | GGUUAUGUUCUGAGUUUAATT |
| | | (SEQ ID NO: 353) | (SEQ ID NO: 354) |
| TOP1-1 | siRNA | ACUGGUUCCGGAUCUUGUCTT | GACAAGAUCCGGAACCAGUTT |
| | | (SEQ ID NO: 355) | (SEQ ID NO: 356) |
| TOP1-2 | siRNA | UACAGUUGAUGAUUAUAUCTT | GAUAUAAUCAUCAACUGUATT |
| | | (SEQ ID NO: 357) | (SEQ ID NO: 358) |
| TOP1-3 | siRNA | UAGCAAUCCUCUCUUUGUGTT | CACAAAGAGAGGAUUGCUATT |
| | | (SEQ ID NO: 359) | (SEQ ID NO: 360) |
| TUBGCP3-1 | siRNA | UGGUGCAAGAAAUUUAUUGTT | CAAUAAAUUUCUUGCACCATT |
| | | (SEQ ID NO: 361) | (SEQ ID NO: 362) |
| TUBGCP3-2 | siRNA | UUUGUAAUGCUCGUUGAAGTT | CUUCAACGAGCAUUACAAATT |
| | | (SEQ ID NO: 363) | (SEQ ID NO: 364) |
| TUBGCP3-3 | siRNA | UCUCGAGUAAACACAGUUGTT | CAACUGUGUUUACUCGAGATT |
| | | (SEQ ID NO: 365) | (SEQ ID NO: 366) |
| USP1-1 | siRNA | UAUAAUACCUGAAGUAUACTT | GUAUACUUCAGGUAUUAUATT |
| | | (SEQ ID NO: 367) | (SEQ ID NO: 368) |
| USP1-2 | siRNA | UAUUGCCGAGAUUAUUCAGTT | CUGAAUAAUCUCGGCAAUATT |
| | | (SEQ ID NO: 369) | (SEQ ID NO: 370) |
| USP1-3 | siRNA | UCUAAUGUAUCACUUGUAGTT | CUACAAGUGAUACAUUAGATT |
| | | (SEQ ID NO: 371) | (SEQ ID NO: 372) |
| UVRAG-1 | siRNA | UUGAUAUUGUCUUUGAUUGTT | CAAUCAAAGACAAUAUCAATT |
| | | (SEQ ID NO: 373) | (SEQ ID NO: 374) |
| UVRAG-2 | siRNA | UCAAGGAAUUCUUAAUCACTT | GUGAUUAAGAAUUCCUUGATT |
| | | (SEQ ID NO: 375) | (SEQ ID NO: 376) |
| UVRAG-3 | siRNA | UGUCAACUGAGCAUUAGUCTT | GACUAAUGCUCAGUUGACATT |
| | | (SEQ ID NO: 377) | (SEQ ID NO: 378) |
| Luciferase | siRNA | UCGAAGUAUUCCGCGUACGdTdT | CGUACGCGGAAUACUUCGAdTdT |
| | | (SEQ ID NO: 11) | (SEQ ID NO: 12) |

Results:

TABLE 6 summarizes the data obtained 66 hours post-transfection by FACS analysis showing the cell cycle distribution of the HCT116DICER^{ex5} cells transfected with siRNA pools of 3 siRNAs/gene. Each pool was analyzed in two separate experiments, and the average percentage of the cells trapped in G1 (following nocodazole treatment), or G2/M (following aphidicolin treatment) are shown in TABLE 6.

TABLE 6
Cell Cycle Analysis of HCT116DICER^{ex5} cells transfected
with pools of three different siRNAs per gene.

| | Nocodazole+ | | Aphidicholin+ | |
|---|---|---|---|---|
| Gene | % G1 (Avg) | STDEV | % G2 (Avg) | STDEV |
| BCL2 | 33.2 | 3.1 | 29.3 | 0.1 |
| BRCA1 | 63.9 | 8.4 | 25.6 | 0.8 |
| CDC14A | 32.0 | 0.2 | 16.5 | 0.2 |
| CDC7 | 39.1 | 0.1 | 17.0 | 2.0 |
| CDC73 | 24.5 | 1.1 | 16.2 | 1.6 |
| CRK | 18.1 | 1.3 | 22.1 | 4.5 |
| CTCF | 25.5 | 3.4 | 20.1 | 3.4 |
| CUL5 | 50.5 | 10.2 | 35.0 | 6.8 |
| DBF4B | 29.8 | 0.5 | 9.7 | 0.2 |
| DEK | 54.2 | 1.0 | 19.7 | 0.4 |
| DLG5 | 46.4 | 8.2 | 30.1 | 3.7 |
| DTL | 20.6 | 15.0 | 39.1 | 13.8 |
| EMP1 | 32.0 | 0.4 | 13.4 | 2.5 |
| ERCC3 | 65.1 | 8.4 | 23.5 | 4.7 |
| EXTL2 | 21.1 | 3.0 | 19.0 | 0.9 |
| FGF2 | 33.7 | 3.3 | 19.8 | 2.2 |
| HOXA10 | 58.5 | 7.0 | 16.4 | 1.6 |
| HRH1 | 51.3 | 1.9 | 15.9 | 3.4 |
| KIF14 | 15.7 | 7.7 | 64.8 | 22.3 |
| KIF23 | 18.4 | 11.3 | 65.0 | 6.3 |
| LMNB2 | 63.1 | 8.0 | 19.4 | 5.0 |
| MACF1 | 37.6 | 0.6 | 18.0 | 0.9 |
| MAD2L1 | 38.8 | 5.0 | 42.6 | 1.7 |
| MCM10 | 29.5 | 9.3 | 25.8 | 3.4 |
| MCM3 | 62.4 | 3.1 | 12.2 | 3.4 |
| MCM6 | 35.6 | 1.3 | 16.9 | 3.2 |
| MIS12 | 64.5 | 3.7 | 15.8 | 5.2 |
| MKI67 | 13.5 | 0.4 | 17.3 | 1.0 |
| MPHOSPH1 | 47.2 | 3.7 | 17.5 | 3.1 |
| MTSS1 | 36.2 | 1.1 | 20.3 | 0.2 |
| NBN | 21.3 | 0.7 | 13.9 | 0.1 |
| NCAPH | 11.4 | 0.3 | 29.0 | 5.3 |
| NEK1 | 28.7 | 1.6 | 16.6 | 0.8 |
| NFIB | 61.5 | 6.8 | 12.2 | 1.1 |
| NFYA | 18.4 | 3.5 | 18.6 | 3.7 |
| NIN | 28.5 | 0.1 | 17.6 | 0.6 |
| PAFAH1B1 | 42.0 | 4.6 | 15.4 | 2.1 |
| PAPD5 | 23.4 | 1.8 | 18.0 | 0.7 |
| PFTK1 | 62.4 | 0.3 | 11.7 | 0.0 |
| PIM1 | 33.1 | 3.9 | 37.1 | 0.7 |
| PLAU | 58.8 | 9.6 | 19.0 | 3.1 |
| PLS3 | 47.9 | 2.1 | 20.3 | 0.9 |
| POLQ | 33.8 | 0.6 | 10.2 | 0.2 |
| POLS | 17.0 | 3.4 | 21.2 | 2.8 |
| PPP1CA | 35.3 | 1.7 | 19.4 | 6.4 |
| PRPF38A | 42.6 | 8.7 | 28.9 | 1.4 |
| RACGAP1 | 10.4 | 5.4 | 57.1 | 25.7 |
| RAD1 | 31.7 | 2.3 | 22.0 | 3.2 |
| RAD21 | 78.2 | 6.0 | 9.7 | 0.2 |
| RAD51 | 54.0 | 5.4 | 29.4 | 5.2 |
| RAP1GAP | 32.2 | 3.1 | 13.8 | 2.5 |
| RB1 | 9.5 | 1.2 | 20.3 | 0.4 |
| SEPT10 | 75.1 | 1.1 | 12.6 | 3.5 |
| SERTAD2 | 43.3 | 4.1 | 15.0 | 0.6 |
| SH3BP4 | 37.0 | 5.9 | 23.2 | 1.7 |
| SMARCB1 | 36.6 | 3.0 | 27.0 | 0.9 |
| TDG | 55.8 | 8.2 | 18.1 | 1.1 |
| TDP1 | 9.9 | 0.8 | 25.0 | 2.5 |
| TOP1 | 32.8 | 2.3 | 13.1 | 4.0 |
| TUBGCP3 | 21.0 | 4.5 | 21.7 | 3.9 |
| USP1 | 42.9 | 4.3 | 23.7 | 2.9 |
| UVRAG | 43.9 | 2.0 | 17.8 | 1.6 |
| MOCK | 8.6 | 0.8 | 18.8 | 0.4 |
| Luc siRNA | 13.8 | 1.8 | 18.9 | 2.7 |
| miR-192 | 43.6 | 5.9 | 54.9 | 7.5 |

As shown above in TABLE 6, silencing of many of the 62 genes by transfecting gene-specific siRNA pools caused some measure of G1 or G2/M arrest, as compared to the control luciferase siRNA-transfected samples.

Of the 62 genes shown above in TABLE 6, the 10 genes whose targeting by siRNA caused the largest percentage of cells to arrest in G1, listed below in TABLE 7, were transfected into HCT116DICER^{ex5} cells in another experiment as follows.

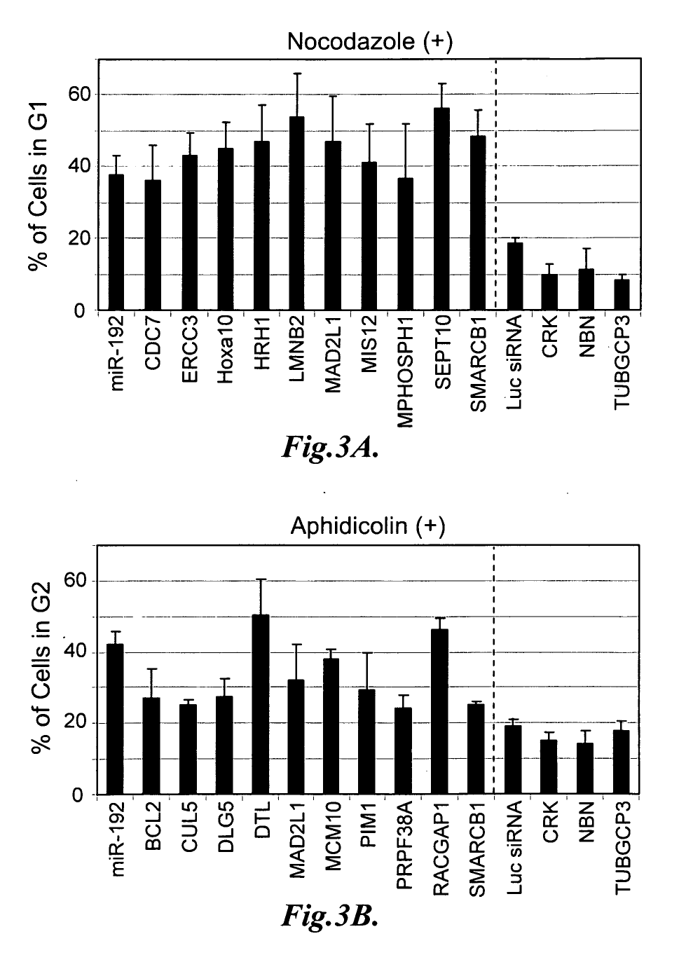

HCT116DICER^{ex5} cells were transfected with 10 mM miR-192 or 100 nM siRNA against luciferase, or 100 nM siRNA (one siRNA duplex of the pool of three) against the putative miR-192 target of interest, as indicated in TABLES 7 and 8. At 48 hours post transfection, cells were treated with nocodazole or aphicicolin for an additional 18 hours prior to FACS analysis. The results for the nocodazole treated cells (% of cells in G1) are provided in TABLE 7, and graphically illustrated in FIG. 3A. The results for the aphidicolin treated cells (% of cells in G2) are provided in TABLE 8 and graphically illustrated in FIG. 3B.

TABLE 7
Target Genes Downregulated by miR-192 that cause a G1 Arrest
Phenotype

| | | Guide Strand/ | % Cells | |
|---|---|---|---|---|
| Gene | Genbank # | Passenger Strand | in G1 | STDDEV |
| SEPT10 | NM_144710 | (SEQ ID NOS: 13/14; 15/16; | 56.3 | 6.7 |
| | | 17/18) | | |
| LMNB2 | NM_032737 | (SEQ ID NOS: 19/20; 21/22; | 54.0 | 12.2 |
| | | 23/24) | | |
| SMARCB1 | NM_003073 | (SEQ ID NOS: 61/62; 63/64; | 48.5 | 7.3 |
| | | 65/66) | | |
| MAD2L1 | NM_002358 | (SEQ ID NOS: 67/68; 69/70; | 47.1 | 12.9 |
| | | 71/72) | | |
| HRH1 | NM_001098213 | (SEQ ID NOS: 25/26; 27/28; | 46.8 | 10.5 |
| | | 29/30) | | |
| Hoxa10 | NM_153715 | (SEQ ID NOS: 31/32; 33/34; | 44.9 | 7.2 |
| | | 35/36) | | |
| ERCC3 | NM_000122 | (SEQ ID NOS: 37/38; 39/40; | 43.2 | 6.3 |
| | | 41/42) | | |
| MIS12 | NM_024039 | (SEQ ID NOS: 43/44; 45/46; | 41.2 | 10.9 |
| | | 47/48) | | |
| miR-192 | | SEQ ID NOS: 1/7 | 37.9 | 5.2 |
| MPHOSPH1 | NM_016195 | (SEQ ID NOS: 49/50; 51/52; | 36.9 | 15.2 |
| | | 53/54) | | |
| CDC7 | hCT2310901 | (SEQ ID NOS: 55/56; 57/58; | 36.4 | 9.4 |
| | | 59/60) | | |
| Luc siRNA | | SEQ ID NOS: 11/12 | 18.5 | 1.6 |
| NBN | NM_002485 | SEQ ID NOS: 223/224; | 11.2 | 5.8 |
| | | 225/226; 227/228 | | |
| CRK | NM_005206 | SEQ ID NOS: 139/140; | 10.0 | 2.8 |
| | | 141/142; 143/144 | | |
| TUBGCP3 | NM_006322 | SEQ ID NOS: 361/362; | 8.3 | 1.4 |
| | | 363/364; 365/366 | | |

Of the 62 genes shown above in TABLE 6, the 10 genes whose targeting by siRNA caused the largest percentage of cells to arrest in G2 is listed below in TABLE 8.

TABLE 8
Target Genes Downregulated by miR-192 that cause a G2 Arrest
Phenotype

| | | Guide Strand/ | % Cells | |
|---|---|---|---|---|
| Gene | Genbank # | Passenger Strand | in G2 | STDDEV |
| DTL | NM_016448 | (SEQ ID NOS: 73/74; | 50.1 | 10.4 |
| | | 75/76; 77/78) | | |
| RACGAP1 | NM_013277 | (SEQ ID NOS: 79/80; | 46.1 | 3.1 |
| | | 81/82; 83/84) | | |
| miR-192 | | SEQ ID NOS: 1/7 | 42.3 | 3.5 |
| MCM10 | NM_018518 | (SEQ ID NO: 85/86; 87/88; | 38.1 | 2.8 |
| | | 89/90) | | |
| MAD2L1 | NM_002358 | (SEQ ID NOS: 67/68; | 32.2 | 9.8 |
| | | 69/70; 71/72) | | |
| PIM1 | NM_002648 | (SEQ ID NOS: 91/92; | 29.4 | 10.3 |
| | | 93/94; 95/96) | | |
| DLG5 | NM_004747 | (SEQ ID NOS: 97/98; | 27.5 | 5.1 |
| | | 99/100; 101/102) | | |
| BCL2 | NM_000633 | (SEQ ID NOS: 103/104; | 26.8 | 8.4 |
| | | 105/106; 107/108) | | |
| CUL5 | NM_003478 | (SEQ ID NOS: 109/110; | 25.4 | 1.3 |
| | | 111/112; 113/114) | | |
| SMARCB1 | NM_003073 | (SEQ ID NOS: 61/62; | 25.0 | 1.2 |
| | | 63/64; 65/66) | | |
| PRPF38A | NM_032864 | (SEQ ID NOS: 115/116; | 24.5 | 3.3 |
| | | 117/118; 119/120) | | |
| Luc siRNA | | SEQ ID NOS: 11/12 | 19.2 | 1.8 |
| TUBGCP3 | NM_006322 | SEQ ID NOS: 361/362; | 17.8 | 3.0 |
| | | 363/364; 365/366 | | |
| CRK | NM_005206 | SEQ ID NOS: 139/140; | 15.0 | 2.2 |
| | | 141/142; 143/144 | | |
| NBN | NM_002485 | SEQ ID NOS: 223/224; | 14.2 | 3.7 |
| | | 225/226; 227/228 | | |

Discussion:

Using gene expression profiling and RNAi-mediated gene silencing, a set of downstream effectors of miR-192/miR-215 have been identified that includes a number of key regulators of DNA synthesis and the G₁ and G₂ cell cycle checkpoints, as described in more detail in EXAMPLE 5 and EXAMPLE 6.

### Example 5

This Example describes the analysis of miR-192 transfected cells in the U-2-OS cell line and confirmation of the results observed in the HCT116DICER^{ex5} cell line.

Methods:

Transcript Analysis

To further confirm that the candidate genes shown in TABLE 7 and TABLE 8 are direct downstream targets of miR-192/miR-215, miR-192 siRNA (10 nM) synthetic duplexes, miR-192 mutant (10 nM) or 10 nM siRNA against luciferase were transfected into the cell line U-2-OS, an osteosarcoma cell line that has wild-type DICER function and a relatively low endogenous level of miR-192/miR-215.

RNA was isolated at 10 hours post-transfection and transcript abundance of the target genes shown in TABLE 7 and TABLE 8 was measured by quantitative PCR. Transcript abundance was measured by Taqman gene expression assay (Applied Biosystems) using hGUS as an internal control. Levels of transcripts were quantified using an ABI Prism 7900HT sequence detection system.

Luciferase Reporter Analysis

In order to test whether miR-192 is regulating these genes through seed sequence-specific recognition of binding sites within their 3′ UTRs, a series of reporter constructs were generated containing the entire natural 3′ UTRs of the 18 candidate genes (BCL2, CDC7, CUL5, DLG5, DTL, ERCC3, HOXA10, HRH1, LMNB2, MAD2L1, MCM10, MIS12, MPHOSPH1, PIM1, PRPF38A, RACGAP1, SEPT10 and SMARCB1) inserted downstream of a luciferase open reading frame (SwitchGear Genomics) to create 3′ UTR luciferase reporter plasmids. U-2-OS cells were transfected first with 10 nM miR-192 or 10 nM miR-192 mutant, and subsequently transfected 4 to 6 hours later with these 3′ UTR reporter plasmids. A renilla luciferase expression plasmid from dual luciferase system (Promega) was used as an internal control. Luciferase activity was measured at 24 hours post-transfection, and quantified relative to renilla luciferase activity.

Western Blot Analysis:

HCT116DICER^{ex5} cells were transfected with 10 nM siRNA against luciferase or 10 nM miR-192 or 10 nM miR-192 mutant, and lysates were prepared at 28 hours or 48 hours post-transfection. For immunoblotting, 30 μg of whole cell lysate extracted in a modified RIPA buffer (150 mM NaCl, 50 mM Tris pH 7.4, 1 mM EDTA, 1% NP-40, 0.1% SDS) was used per sample. CDC7, LMNB2, MAD2L1 and CUL5 were detected by Western blot using anti-CDC7 (sc-56275, Santa Cruz Biotechnology Inc.), anti-LMNB2 (MAB3536, Millipore), anti-MAD2L1 (ab55452, Abcam) and anti-CUL5 (Invitrogen) antibodies, and protein levels were compared to the level of β-actin expression as detected by an anti-β-actin antibody (Abcam).

Results:

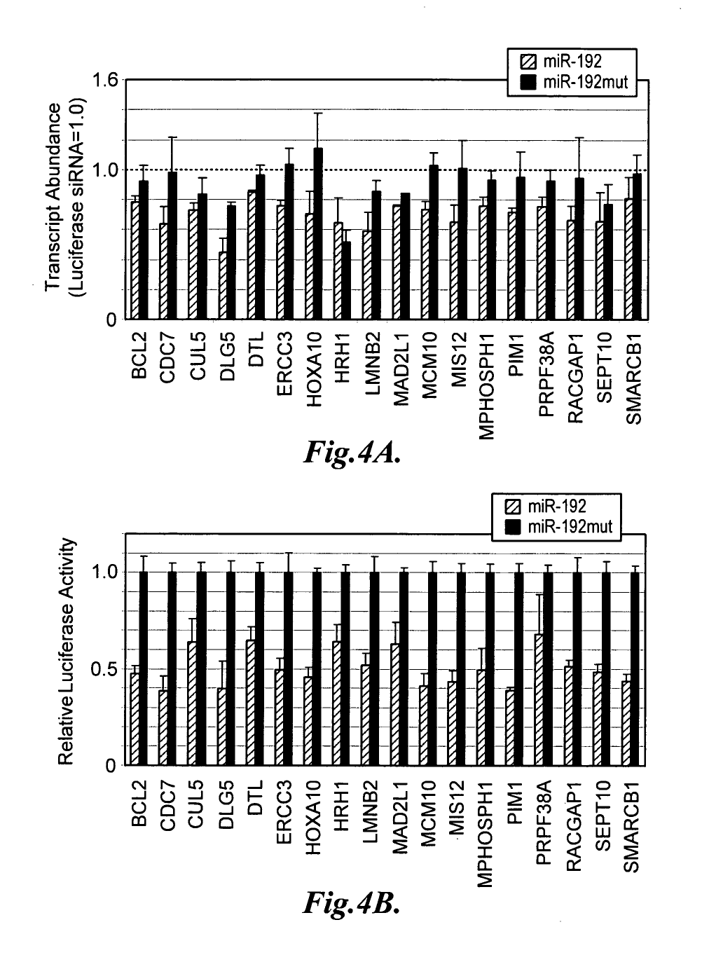

FIG. 4A graphically illustrates the transcript abundance (relative to a control luciferase siRNA) of the set of 18 candidate downstream targets of miR-192/miR-215 in U-2-OS cells transfected with miR-192 or a miR-192 with a seed region mutation. All transcript levels were normalized relative to the abundance of hGUS transcripts. The relative abundance of each gene shown in FIG. 4A following transfection with luciferase siRNA has been set to “1.”

As shown in FIG. 4A, a decrease in candidate gene transcript levels was observed in U-2-OS cells as early as 10 hours following transfection with a miR-192 duplex (SEQ ID NOS:1 and 7), relative to gene transcript levels in cells transfected with either a luciferase siRNA control duplex (SEQ ID NOS:11 and 12) or a miR-192 seed region mutant control duplex (SEQ ID NOS:8 and 9). These results are consistent with the microarray data described in Example 2 and TABLE 3, and confirm the knockdown results obtained in the transfected HCT116DICER^{ex5} cells.

FIG. 4B graphically illustrates the average normalized luciferase activity for each cell co-transfected with a reporter construct containing the 3′ UTR of a candidate gene fused to the luciferase open reading frame and with either an miR-192 or miR-192 seed mutant, as measured in three separate trials conducted in duplicate. FIG. 4B represents the average normalized luciferase activity as measured in three separate experiments conducted in duplicate. For each reporter construct, the luciferase activity of samples transfected with miR-192 mutant is set to a value of “1.” As shown in FIG. 4B, 3′ UTRs from these 18 genes were regulated by miR-192 but not by the miR-192 mutant, indicating that these 3′ UTRs are sufficient to confer regulation of a heterologous reporter gene (luciferase) by miR-192. It was also determined by Western blot analysis that the protein level of proteins carrying the miR-192, but not the miR-192 seed region mutant, were also downregulated (data not shown).

These results in the U-2-OS cell line confirm the knockdown results obtained in the transfected HCT116DICER^{ex5} cells and further demonstrate that the 3′ UTRs of the set of 18 genes identified as downstream targets of miR-192/miR-215 are sufficient to confer regulation of a heterologous reporter gene (luciferase) by miR-192.

### Example 6

This Example demonstrates that a pool of siRNAs targeting a set of miR-192 downstream targets is effective to phenocopy the cell cycle effects of miR-192 when transfected at sub-optimal concentrations.

Rationale: As described above in EXAMPLES 1-5, a set of miR-192/miR-215 regulated genes have been identified that, when targeted by siRNA, individually reproduce the miR-192 cell cycle arrest phenotype described in EXAMPLE 3. It was observed that miR-192 down-regulated these target transcripts to a lesser degree (30-40% down-regulation) than the target-specific siRNAs that were used (approximately 80% down-regulation). Therefore, it was hypothesized that miR-192 induced cell cycle arrest might arise from the coordinated regulation of multiple cell cycle-related transcripts in this network of downstream targets. In order to test this hypothesis, a pool of siRNAs targeting the identified miR-192 downstream targets was transfected into cells at sub-optimal concentrations to see if this would phenocopy the cell cycle effects of enforced expression of miR-192.

1. Titration of siRNA Concentration of miR-192 to Determine Sub-Optimal Levels for Inducing Cell Cycle Phenotypes

Methods:

HCT116DICER^{ex5} cells were transfected with 10 nM miR-192 or 100 nM siRNA against luciferase, or 100 nM, 10 nM, 1 nM, 0.1 nM or 0.01 nM siRNA against the respective miR-192 target genes of interest, as shown in TABLE 9 and TABLE 10. At 48 hours post-transfection, cells were treated with nocodazole or aphidicolin for an additional 18 hours prior to FACS analysis.

Results:

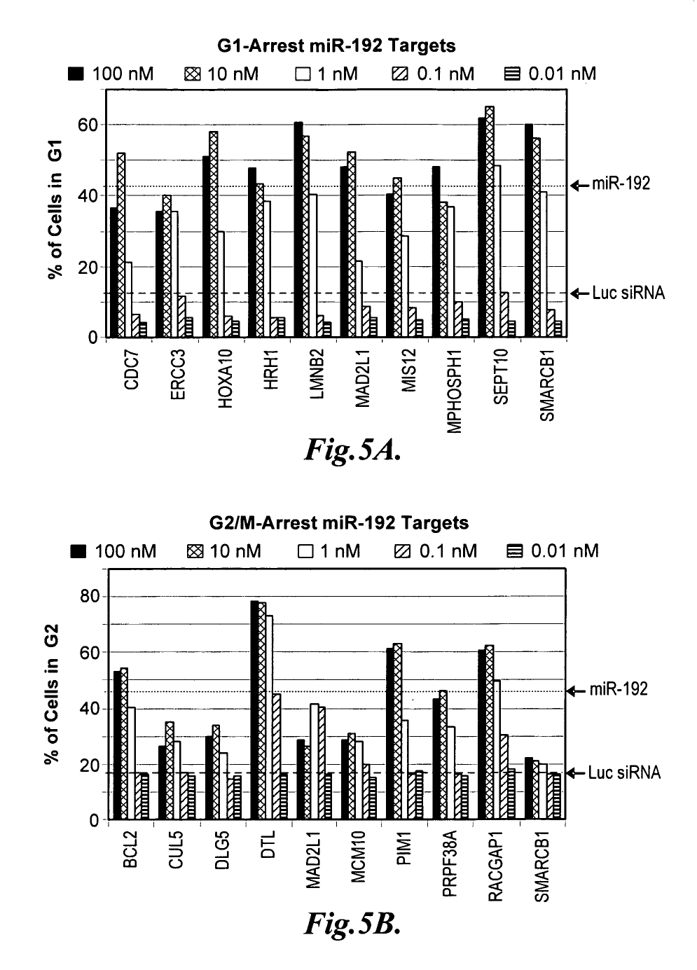

FIG. 5A graphically illustrates the titration of siRNAs targeting miR-192 responsive genes in HCT116DICER^{ex5} cells after treatment with nocodazole that phenocopy miR-192 induced G1 arrest. The percentage of cells arrested in G1 of the total events analyzed in each sample is shown in FIG. 5A. The horizontal line labeled “miR-192” represents the percentage of cells that were arrested in G1 following miR-192 transfection, while the horizontal line labeled “Luc siRNA” represents the percentage of cells that were arrested in G1 following luciferase siRNA transfection. Concentration levels of siRNA resulting in G1 arrest that fell at or below the levels of G1 arrest induced by luciferase siRNA (i.e., sub-optimal concentrations) were used to create siRNA pools for further analysis.

FIG. 5B graphically illustrates the titration of siRNAs targeting miR-192 responsive genes in HCT116DICER^{ex5} cells after treatment with aphidicolin that phenocopy miR-192 induced G2 arrest. The percentage of cells that were arrested in G2 of the total events analyzed in each sample is shown in FIG. 5B. The horizontal line labeled “miR-192” represents the percentage of cells that were arrested in G2 following miR-192 transfection, while the horizontal line labeled “Luc siRNA” represents the percentage of cells that were arrest in G2 arrested following luciferase siRNA transfection. Concentration levels of siRNA resulting in G2 arrest that fell at or below the levels of G2 arrest induced by luciferase siRNA (i.e., sub-optimal concentrations) were used to create siRNA pools for further analysis.

Based on the results shown in FIGS. 5A and 5B, it was determined that transfection of siRNAs at 0.1 to 0.01 nM did not cause G1 or G2/M arrest.

2. Transfection of siRNA Pools at Suboptimal Levels to Determine Whether Such siRNA Pools could Recapitulate the miR-192 Induced Cell Cycle Phenotypes

Methods:

Two siRNA pools for the G1 gene set shown in TABLE 9 were constructed consisting of siRNAs, one pool with each siRNA represented at a final concentration of 0.1 nM, and a second pool with each siRNA represented at a final concentration of 0.01 nM. At 48 hours post-transfection into HCT116DICER^{ex5} cells, the cells were treated with nocodazole for an additional 18 hours prior to FACS analysis.

TABLE 9
siRNA pool for G1 Gene Set

| Target Genes | | |
|---|---|---|
| for G1 Arrest | | |
| Phenotype | Genbank | siRNA synthetic duplexes |
| CDC7 | hCT2310901 | (SEQ ID NOS: 55/56; 57/58; 59/60) |
| ERCC3 | NM_000122 | (SEQ ID NOS: 37/38; 39/40; 41/42) |
| HOXA10 | NM_153715 | (SEQ ID NOS: 31/32; 33/34; 35/36) |
| HRH1 | NM_001098213 | (SEQ ID NOS: 25/26; 27/28; 29/30) |
| LMNB2 | NM_032737 | (SEQ ID NOS: 19/20; 21/22; 23/24) |
| MAD2L1 | NM_002358 | (SEQ ID NOS: 67/68; 69/70; 71/72) |
| MIS12 | NM_024039 | (SEQ ID NOS: 43/44; 45/46; 47/48) |
| MPHOSPH1 | NM_016195 | (SEQ ID NOS: 49/50; 51/52; 53/54) |
| SEPT10 | NM_144710 | (SEQ ID NOS: 13/14; 15/16; 17/18) |
| SMARCB1 | NM_003073 | (SEQ ID NOS: 61/62; 63/64; 65/66) |

Two siRNA pools for the G2 gene set shown in TABLE 10 were constructed consisting of siRNAs, one pool with each siRNA represented at a final concentration of 0.1 nM, and a second pool with each siRNA represented at a final concentration of 0.01 nM. At 48 hours post-transfection into HCT116DICER^{ex5} cells, the cells were treated with aphidicolin for an additional 18 hours prior to FACS analysis.

TABLE 10
siRNA pool for G2 Gene Set

| Target Genes | | |
|---|---|---|
| for G2 | | |
| Arrest | | |
| Phenotype | Genbank | siRNA synthetic duplexes |
| BCL2 | NM_000633 | (SEQ ID NOS: 103/104; 105/106; 107/108) |
| CUL5 | NM_003478 | (SEQ ID NOS: 109/110; 111/112; 113/114) |
| DLG5 | NM_004747 | (SEQ ID NOS: 97/98; 99/100; 101/102) |
| DTL | NM_016448 | (SEQ ID NOS: 73/74; 75/76; 77/78) |
| MAD2L1 | NM_002358 | (SEQ ID NOS: 67/68; 69/70; 71/72) |
| MCM10 | NM_018518 | (SEQ ID NOS: 85/86; 87/88; 89/90) |
| PIM1 | NM_002648 | (SEQ ID NOS: 91/92; 93/94; 95/96) |
| PRPF38A | NM_032864 | (SEQ ID NOS: 115/116; 117/118; 119/120) |
| RACGAP1 | NM_013277 | (SEQ ID NOS: 79/80; 81/82; 83/84) |
| SMARCB1 | NM_003073 | (SEQ ID NOS: 61/62; 63/64; 65/66) |

TABLE 11
Cell cycle distribution of miR-192 or siRNA transfected
HCT116DICER^{ex5} cells after Nocodazole treatment

| siRNA Transfected | % G1 | % G2 |
|---|---|---|
| Luciferase siRNA | 11.7 | 83.8 |
| control (100 nM) | | |
| miR-192 (10 nM) | 43.9 | 53.6 |
| siRNA G1 gene pool | 38.5 | 29.7 |
| (0.1 nM) | | |
| siRNA G1 gene pool | 10.4 | 70.7 |
| (0.01 nM) | | |

TABLE 12
Cell cycle distribution of miR-192 or siRNA transfected
HCT116DICER^{ex5} cells after aphidicolin treatment

| siRNA Transfected | % G1 | % G2 |
|---|---|---|
| Luciferase siRNA control | 66.3 | 15.6 |
| (100 nM) | | |
| miR-192 (10 nM) | 50.4 | 44.7 |
| siRNA G2 gene pool | 26.3 | 60.3 |
| (0.1 nM) | | |
| siRNA G2 gene pool | 58.3 | 19.1 |
| (0.01 nM) | | |

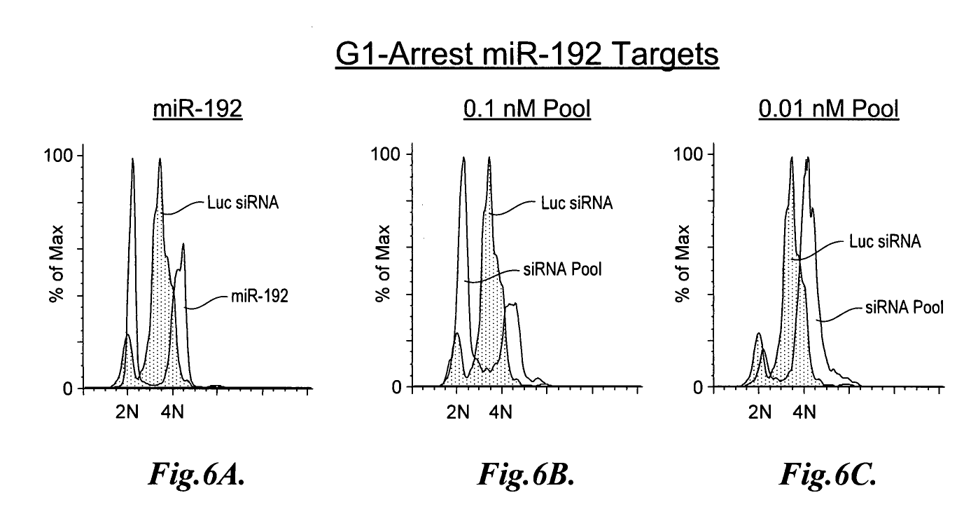

As shown in TABLE 11, cell cycle distribution of luciferase siRNA transfected cells was compared to miR-192 or siRNA G1 gene pool transfected cells at 66 hours post transfection. As demonstrated in TABLE 11, the siRNA pool targeting the G1 specific miR-192 target genes phenocopied the miR-192 induced G1 arrest. These results are graphically illustrated in FIG. 6A-6C. As shown in FIG. 6A, transfection of miR-192 followed by treatment with nocodazole induced a G1-arrest phenotype, which was phenocopied with the siRNA G1 gene pool transfected at 0.1 nM (shown in FIG. 6B). As shown in FIG. 6C, the siRNA G1 gene pool transfected at 0.01 nM did not result in the G1 arrest phenotype.

As shown in TABLE 12, cell cycle distribution of luciferase siRNA transfected cells was compared to miR-192 or siRNA G2 gene pool transfected cells at 66 hours post transfection. As demonstrated in TABLE 12, the siRNA pool targeting the G2 specific miR-192 target genes phenocopied the miR-192 induced G2 arrest.

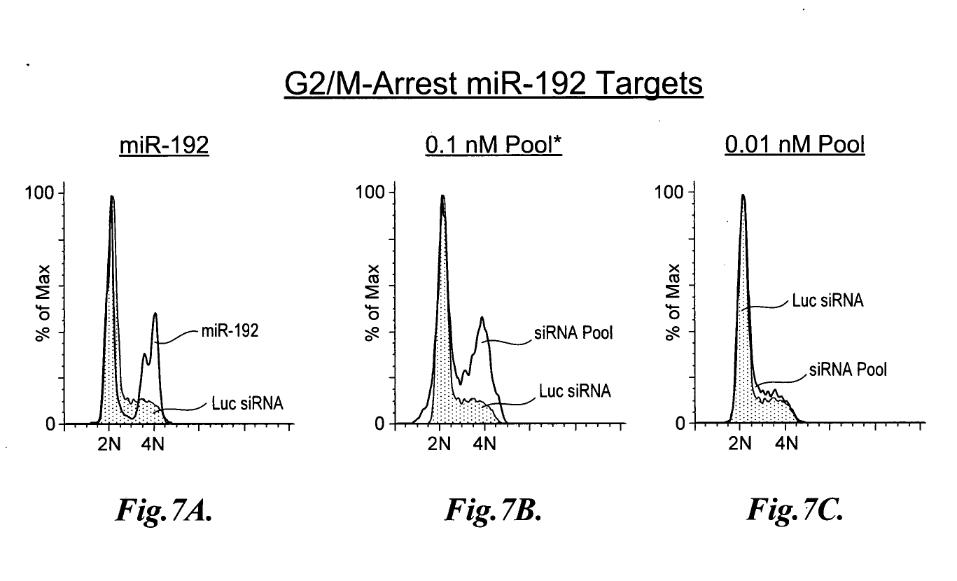

These results are graphically illustrated in FIG. 7A-7C. As shown in FIG. 7A, transfection of miR-192 followed by treatment with aphidicolin induced a G2-arrest phenotype, which was phenocopied with the siRNA G2 gene pool transfected at 0.1 nM (shown in FIG. 7B). As shown in FIG. 7C, the siRNA G2 gene pool transfected at 0.01 nM did not result in the G2 arrest phenotype.

Discussion:

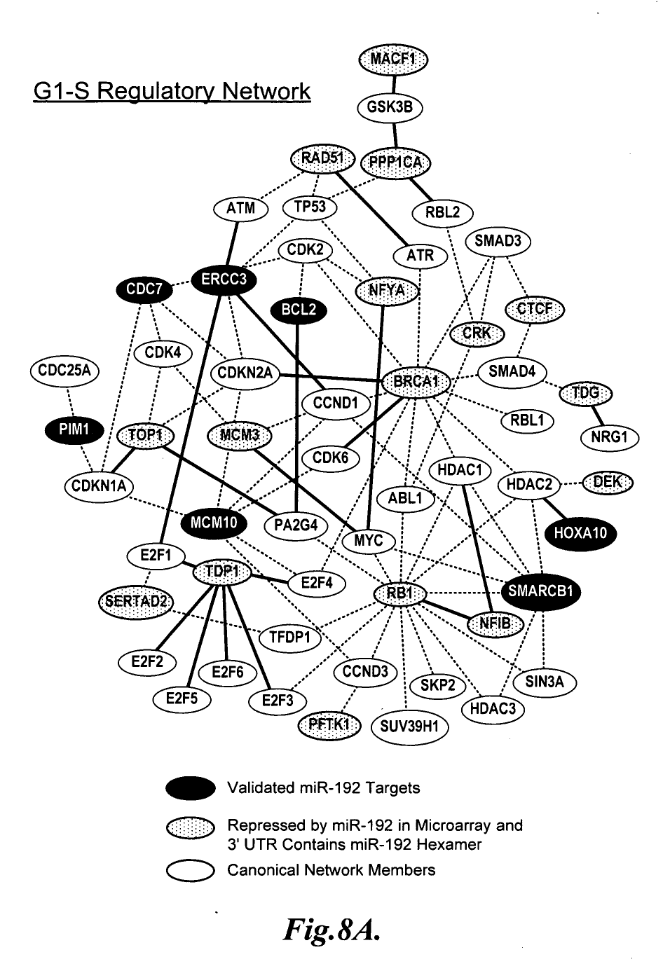

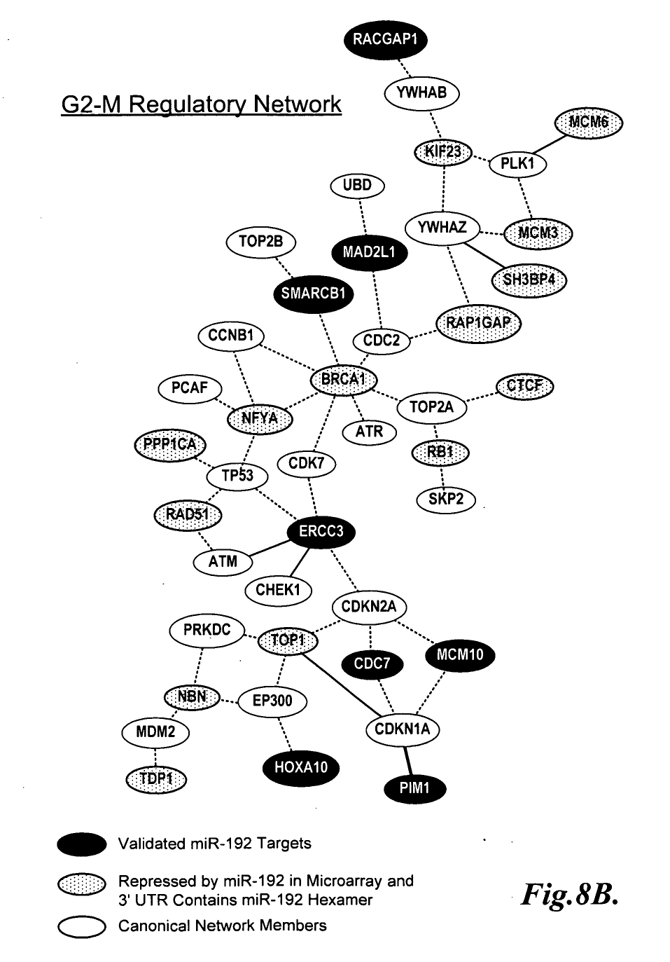

These results demonstrate that miR-192/miR-215 regulates cell cycle progression by regulating the expression of key cell cycle genes. By simultaneously regulating the expression of these key cell cycle genes, miR-192/miR-215 may mediate the cell cycle arrest function of p53. It has been shown that microRNAs may influence cellular processes through coordinate regulation of many targets (Linsley, P. S., et al., Mol. Cell Biol. 27:2240-2252 (2007); Lim, L. P., et al., Nature 433:769-773 (2005)). In this study we have demonstrated that miR-192/miR-215 act to halt cell cycle progression by coordinately targeting transcripts that play critical roles in mediating the G₁/S and G₂/M checkpoints. Significantly, the regulatory signature of miR-192/miR-215 (as shown in TABLE 3) overlaps substantially with canonical G₁/S (FIG. 8A) and canonical G₂/M (FIG. 8B) cell cycle checkpoint networks.

FIG. 8A is a diagram of the canonical G1-S cell cycle checkpoint network, illustrating the members of the network found to be regulated by miR-192/miR-215 by microarray analysis (shown as black ovals) and the members of the network that were confirmed to be direct miR-192/miR-215 targets (shown as hatched ovals). FIG. 8B is a diagram of the canonical G2-M cell cycle checkpoint network, illustrating the members of the network found to be regulated by miR-192/miR-215 by microarray analysis (shown as black ovals) and the members of the network that were confirmed to be direct miR-192/miR-215 targets (shown as hatched ovals). The cell cycle networks shown in FIGS. 8A and 8B were constructed using interactions between G1-S and G2-M checkpoint genes defined in the Ingenuity Pathways Analysis database (Ingenuity Systems®, www.ingenuity.com) and the miR-192 repression signature. The edges were derived from protein-protein interactions (PPIs) defined in the following databases: BIND (Bader, G. D., et al., Nucleic Acis Res 31:248-250 (2003); BioGRID (Breitkreutz, B. J., et al., Nucleic Acids Res. 36:D637-640 (2008); DIP (Salwinski, L., et al., Nucleic Acids Res 32:D449-451 (2004); HPRD (Mishra, G. R., et al., Nucleic Acis Res 34:D411-414 (2006); MINT (Chatr-aryamontri, A., et al., Nucleic Acis Res 35:D572-574 (2007); NetPro, Proteome (BioBase www.proteome.com); Reactome (Joshi-Tope, G., et al., Nucleic Acids Res 33:D428-432 (2005); Ingenuity; and GeneGo MetaBase (GeneGo www.genego.com). The solid edges between the nodes in the pathways illustrated in FIG. 8A and FIG. 8B indicate protein-protein interactions, as defined in the following databases: BIND, BioGRID, DIP, HPRD, MINT, NetPro, Proteome, Reactome, Ingenuity and GeneGo. In cases where the same PPI edge was represented in multiple data sources, the edges were collapsed into a single edge to improve visualization (dotted edges).

Consistent with this notion, as demonstrated in Example 3, the enforced expression of miR-192 led to cell cycle arrest in the G₁ and G₂/M phases of the cell cycle. While not wishing to be bound by theory, it is believed that miR-192/miR-215 likely contributes to p53-induced cell cycle arrest by regulating the expression of these key cell cycle transcripts. As demonstrated in Example 2, gene expression profiling of miR-192/miR-215 expressing cells identified a set of 62 transcripts that contain hexamer sequences complementary to an miR-192/miR-215 seed region in their 3′ UTRs. Of these transcripts, 18 transcripts are direct targets of miR-192/miR-215, as demonstrated in Example 5 and FIG. 4B. As expected, individually down-regulating these putative miR-192/miR-215 targets by potent siRNA duplexes resulted in cell cycle arrest, as described in Example 4. However, the level of suppression of these genes by siRNA exceeded the level of suppression that was observed by miR-192 targeting, as shown in TABLE 7 and TABLE 8. It was also determined that individually administered siRNA concentrations that mimicked the level of miR-192 suppression were inadequate to suppress cell cycle progression (data not shown). Instead, as demonstrated in this Example, by siRNA pooling experiments we found that simultaneous subtle modulation (<40% decrease of target transcripts) of miR-192 targets phenocopied the miR-192/miR-215 cell cycle effect, as shown in FIGS. 6A-C and FIGS. 7A-C. Therefore, the observed cell cycle arrest likely results from a cooperative effect among the modulations of a plurality of these genes by miR-192/miR-215. Taken together, these results demonstrate that miR-192/miR-215 expression induces cell cycle arrest by cooperatively targeting multiple cell cycle transcripts.

Among the miR-192/miR-215 targets identified in TABLE 3, there are genes that are essential for the progression of the cell cycle. For example, CDC7 and the MCM proteins are required for the initiation of DNA synthesis and S phase progression. MCM10 has been shown to be required for the recruitment of the MCM2-7 DNA helicase complex as well as DNA polymerase-α to replication origins at the initiation of DNA synthesis, and the mutation of MCM10 in yeast has been shown to cause the accumulation of replication forks in S phase (Maiorano, D., et al., Curr. Opin. Cell Biol. 18:130-136 (2006); Ricke, R., et al., Mol. Cell 16:173-185 (2004); Homesley, L., et al., Genes Dev. 14:913-926 (2000)). In addition to MCM10, MCM3 and MCM6 also contain miR-192 hexamers in their 3′ UTRs and were down-regulated by miR-192 in the microarray experiment (see TABLE 3). The CDC7 kinase is also known to be a participant in the initiation of DNA replication, since its phosphorylation of MCM2 and MCM4 upon the recruitment of these proteins to the replication origins is important for initiating DNA synthesis (Woo, R. A., et al., Cell Cycle 2:316-324 (2003); Masai, H., et al., J. Biol. Chem. 275:29-42-29052 (2000); Lei, M., et al., Genes Dev. 11:3365-3374 (1997)).

While not wishing to be bound by theory, it is believed that in addition to regulating cell cycle-related genes directly, miR-192 could also induce arrest through targeting genes that consequently activate the p53-p21 pathway. For example, suppression of DTL by miR-192 may promote p53 stabilization as DTL has been shown to interact with both the DDB1-CUL4 and MDM2-p53 complexes to destabilize p53 (Banks, D., et al., Cell Cycle 5:1719-1729 (2006); Higa, L. A., et al., Cell Cycle 5:1675-1680 (2006)). Furthermore, miR-192-mediated suppression of CDC7 may induce p21 (Kim, J. M., et al., EMBO J. 21:2168-2179 (2002)), providing an additional mechanistic explanation for how miR-192 may function in the p53 pathway. Taken together, the results described herein suggest that p53 and miR-192/miR-215 act together to coordinate the transcriptional and post-transcriptional events that mediate cell cycle arrest following exposure to genotoxic stress.

Consistent with these results, recent microarray analyses of colon adenocarcinomas found that miR-192/miR-215 expression is significantly reduced in tumor samples relative to matched adjacent noninvolved tissue (Schetter, A. J., et al., JAMA 299:425-436 (2008)). Interestingly, several of the transcripts identified in TABLE 3 as miR-192/miR-215 targets have been reported as being over-expressed in tumors, including DTL over-expression in aggressive liver cancer (Pan, H. W., et al., Cell Cycle 5:2676-2687 (2006)), and CDC7 up-regulation in endocrine tumors, thyroid tumors, melanomas, and head and neck squamous cell carcinomas (Mould, A. W., et al., Int. J. Cancer 121:776-783 (2007); Slebos, R. J., et al., Clin. Cancer Res. 12:701-709 (2006); Kaufman, W. K., et al., J. Invest. Dermatol. 128:175-187 (2008); Fluge, O., et al., Thyroid 16:161-175 (2006)).

Therefore, these results demonstrate a role for miR-192/miR-215 in cell proliferation, which, combined with recent observations that these miRNAs are under-expressed in primary cancers (Schetter, A. J., et al., JAMA 299:425-436 (2008)), support the conclusion that miR-192 and miR-215 function as tumor-suppressors.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method of inhibiting proliferation of a mammalian cell comprising introducing into the mammalian cell an effective amount of at least one small interfering nucleic acid (siNA) agent that inhibits the level of expression of at least one miR-192 family responsive gene comprising SEQ ID NO:379 in its 3′ untranslated region (3′UTR).

2. The method of claim 1, wherein the at least one miR-192 family responsive gene is selected from TABLE 3.

3. The method of claim 1, wherein the at least one siNA agent comprises a guide strand contiguous nucleotide sequence of at least 18 nucleotides, and a passenger strand, wherein said guide strand comprises a seed region consisting of nucleotide positions 1 to 12, wherein position 1 represents the 5′ end of said guide strand and wherein said seed region comprises a nucleotide sequence of at least six contiguous nucleotides that is identical to six contiguous nucleotides within a sequence selected from the group consisting of SEQ ID NO:3 and SEQ ID NO:6.

4. The method of claim 3, wherein said guide strand contiguous nucleotide sequence consists of 22 nucleotides and said seed region consists of nucleotide positions 1 to 12.

5. The method of claim 4, wherein the seed region comprises a nucleotide sequence that is identical to SEQ ID NO:3 or SEQ ID NO:6.

6. The method of claim 1, wherein said siNA further comprises a non-nucleotide moiety.

7. The method of claim 3, wherein the guide strand is stabilized against nucleolytic degradation.

8. The method of claim 3, wherein the siNA further comprises at least one chemically modified nucleotide or non-nucleotide at the 5′ end and/or the 3′ end of the guide strand and the 3′ end of the passenger strand.

9. The method of claim 3, wherein the passenger strand of the at least one siNA agent comprises a nucleic acid molecule consisting of a nucleotide sequence of 18 to 25 nucleotides, said passenger strand comprising a nucleotide sequence that has at least one nucleotide sequence difference compared with the true reverse complement sequence of the seed region of the guide strand, wherein the at least one nucleotide difference is located within nucleotide position 13 to the 3′ end of said passenger strand.

10. The method of claim 3, wherein siNA further comprises one 3′ overhang wherein said 3′ overhang consists of 1 to 4 nucleotides.

11. The method of claim 3, wherein said siNA further comprises a phosphorothioate located at least one of the first internucleotide linkage at the 5′ end of the passenger strand and guide strand and the first internucleotide linkage at the 3′ end of the passenger strand and the guide strand.

12. The method of claim 3, wherein the siNA further comprises a 2′-modified nucleotide.

13. The method of claim 12, wherein the 2′-modified nucleotide comprises a modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA).

14. The method of claim 1, comprising introducing an effective amount of at least one siNA agent that inhibits the expression of at least one miR-192 responsive gene selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

15. The method of claim 1, wherein the at least one siNA agent is a gene-specific inhibitor of expression of at least one miR-192 responsive gene selected from TABLE 3.

16. The method of claim 15, wherein the at least one siNA agent comprises a plurality of pools of siRNA molecules directed against at least two miR-192 responsive genes selected from TABLE 3.

17. The method of claim 16, comprising introducing a plurality of pools of siRNA molecules directed against at least two miR-192 responsive genes selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

18. The method of claim 15, wherein the at least one gene-specific siNA agent comprises a dsRNA molecule comprising one nucleotide strand that is substantially identical to a portion of the mRNA encoding at least one of the genes selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

19. The method of claim 15, wherein the at least one gene-specific siNA agent comprises a ssRNA molecule comprising one nucleotide strand that is substantially complementary to a portion of the mRNA encoding at least one of the genes selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

20. The method of claim 15, wherein the at least one gene-specific siNA agent is at least one dsRNA molecule comprising a double-stranded region, wherein one strand of the double-stranded region is substantially identical to 15 to 25 consecutive nucleotides encoding a gene selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A, and the second strand is substantially complementary to the first, and wherein at least one end of the dsRNA has an overhang of 1 to 4 nucleotides.

21. The method of claim 1, wherein the siNA agent comprises at least one dsRNA molecule comprising at least one of SEQ ID NO:13 to SEQ ID NO:120.

22. The method of claim 1, wherein the mammalian cell is a cancer cell.

23. A method of inhibiting cancer cell proliferation in a subject comprising contacting the cancer cells with an effective amount of at least one small interfering nucleic acid (siNA) agent that inhibits the level of expression of at least two miR-192 family responsive genes selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A, thereby inhibiting the proliferation of cancer cells in the subject.

24. The method of claim 23, wherein the cancer cell is selected from the group consisting of colon cancer cells, osteosarcoma cells, liver cancer cells, melanoma cancer cells and head and neck squamous cell carcinoma.

25. The method of claim 23, wherein the siNA further comprises a non-nucleotide moiety.

26. The method of claim 23, wherein the siNA comprises a guide strand contiguous nucleotide sequence of at least 18 nucleotides, wherein said guide strand comprises a seed region consisting of nucleotide positions 1 to 12, wherein position 1 represents the 5′ end of said guide strand and wherein said seed region comprises a nucleotide sequence of at least six contiguous nucleotides that is identical to six contiguous nucleotides within a sequence selected from the group consisting of SEQ ID NO:3 and SEQ ID NO:6.

27. The method of claim 23, wherein the siNA comprises a plurality of pools of siRNA molecules.

28. A composition comprising a combination of gene-specific agents directed to at least two miR-192 family responsive target genes selected from TABLE 3.

29. The composition of claim 28, wherein the miR-192 family responsive genes are selected from the group consisting of SEPT10, LMNB2, HRH1, HOXA10, ERCC3, MIS12, MPHOSPHI1, CDC7, SMARCB1, MAD2L1, DTL, RACGAP1, MCM10, PIM1, DLG5, BCL2, CUL5, and PRPF38A.

30. The composition of claim 29, wherein the composition comprises at least one of SEQ ID NO:13 to SEQ ID NO:120.

31. An isolated dsRNA molecule comprising one nucleotide strand that is substantially identical to a sequence selected from the group consisting of SEQ ID NO:13 to SEQ ID NO:120.

32. The isolated dsRNA molecule of claim 31, comprising at least one of SEQ ID NO:13 to SEQ ID NO:120.

33. The isolated dsRNA molecule of claim 31, consisting of at least one of SEQ ID NO:13 to SEQ ID NO:120.

34. A composition comprising at least one synthetic duplex microRNA mimetic and a delivery agent, the synthetic duplex microRNA mimetic(s) comprising:
(i) a guide strand nucleic acid molecule consisting of a nucleotide sequence of 18 to 25 nucleotides, said guide strand nucleotide sequence comprising a seed region nucleotide sequence and a non-seed region nucleotide sequence, said seed region consisting essentially of nucleotide positions 1 to 12 and said non-seed region consisting essentially of nucleotide positions 13 to the 3′ end of said guide strand, wherein position 1 of said guide strand represents the 5′ end of said guide strand, wherein said seed region further comprises a consecutive nucleotide sequence of at least 6 nucleotides that is identical in sequence to a nucleotide sequence selected from the group consisting of SEQ ID NO:3 and SEQ ID NO:6; and
(ii) a passenger strand nucleic acid molecule consisting of a nucleotide sequence of 18 to 25 nucleotides, said passenger strand comprising a nucleotide sequence that has at least one nucleotide sequence difference compared with the true reverse complement sequence of the seed region of the guide strand, wherein the at least one nucleotide difference is located within nucleotide position 13 to the 3′ end of said passenger strand.

35. The composition of claim 34, wherein said guide strand sequence is selected from the group consisting of miR-192 (SEQ ID NO:1) and miR-215 (SEQ ID NO:4).

36. The composition of claim 34, wherein said passenger strand sequence is selected from the group consisting of SEQ ID NO:7 and SEQ ID NO:10.

37. The composition of claim 34, wherein the delivery agent comprises lipid nanoparticles.
